(19) United States
(12) Patent Application Publication
(10) Pub. No.: US 2002/0155472 A1
(43) Pub. Date: Oct. 24, 2002

(54) Glucose transport-related genes and uses thereof

(76) Inventors: Michael P. Czech, Westborough, MA (US); Andrew D. Cherniack, Worcester, MA (US); Adilson L. Guilherme, Shrewsbury, MA (US)

Correspondence Address: J. PETER FASSE, Fish & Richardson P.C., 225 Franklin Street, Boston, MA, 02110-2804, US

(21) Appl. No.: 10/021,162

(22) Filed: Oct. 22, 2001

Related U.S. Application Data
(60) Provisional application No. 60/242,379, filed on Oct. 20, 2000.

(51) Int. Cl. C12Q 1/68; C12M 1/34

(52) U.S. Cl. 435/6; 435/287.2

(57) ABSTRACT

Nucleotide sequences and amino acid sequences from nucleic acids and proteins involved in glucose transport are disclosed. The sequences are useful for producing DNA arrays that can be used for the diagnosis of, predictive testing for, and development of treatments for disorders involving glucose transport such as type II diabetes.

### CROSS-REFERENCE TO RELATED APPLICATIONS

This application claims priority from provisional U.S. application Ser. No. 60/242,379 filed on Oct. 20, 2000, which is herein incorporated by reference in its entirety.

### STATEMENT AS TO FEDERALLY SPONSORED RESEARCH

Work on this invention was supported in part with funds from the Federal government. The government therefore has certain rights in the invention.

### TECHNICAL FIELD

This invention relates to molecular biology, cell biology, glucose transport, medicine, and type II diabetes.

### BACKGROUND

Insulin stimulates glucose transport in muscle and fat. One of the most critical pathways that insulin activates is the rapid uptake of glucose from the circulation in both muscle and adipose tissue. Most of insulin's effect on glucose uptake in these tissues is dependent on the insulin-sensitive glucose transporter, GLUT4 (reviewed in Czech and Corvera, 1999, J. Biol. Chem. 274:1865-1868, Martin et al., 1999, Cell Biochem. Biophys. 30:89-113, Elmendorf et al., 1999 Exp. Cell Res. 253:55-62). The mechanism of insulin action is impaired in diabetes, leading to less glucose transport into muscle and fat. This is thought to be a primary defect in type II diabetes. Potentiating insulin action has a beneficial effect on type II diabetes. This is believed to be the mechanism of action of the drug Rezulin (troglitazone).

Type II diabetes mellitus (non-insulin-dependent diabetes) is a group of disorders, characterized by hyperglycemia that can involve an impaired insulin secretory response to glucose and insulin resistance. One effect observed in type II diabetes is a decreased effectiveness of insulin in stimulating glucose uptake by skeletal muscle. Type II diabetes accounts for about 85-90% of all diabetes cases. In some cases of type II diabetes the underlying physiological defect appears to be multifactoral.

### SUMMARY

The invention is based on the discovery of hundreds of genes that are preferentially expressed in cell types in which glucose transport is affected in type II diabetes, i.e., skeletal muscle and adipose tissue, as well as certain proteins expressed in glucose-transporting vesicles. Accordingly, the invention features methods of identifying a gene whose expression is altered in a glucose transport-related disease or disorder such as type II diabetes.

The invention includes a method of identifying a gene whose expression is altered in a glucose transport-related disorder. The method includes the steps of providing a nucleic acid array having 4 or more nucleic acids immobilized on a solid support, each nucleic acid having a sequence of 10 or more consecutive nucleotides within any one of the sequences listed in FIGS. 1, 2A-2R, 3A-3E, 6A-6E, 7A-7U, 8A-8I, 9, 13A-13C, and complement thereof; providing a reference nucleic acid sample prepared from a tissue of a normal, control mammal; contacting the array with the reference sample; detecting hybridization of the reference sample with nucleic acids in the array, to obtain a reference pattern of glucose transport-related gene expression; providing a test nucleic acid prepared from a tissue of a mammal having a glucose transport-related disorder; contacting the array with the test sample; detecting hybridization of the test nucleic acid with nucleic acids in the array, to obtain a test pattern of glucose transport-related gene expression; and comparing the reference pattern with the test pattern to detect a gene whose expression is altered in the test pattern relative to its expression in the reference pattern. FIGS. 6A-6E, 7A-7U, 8A-8I, 9, 13A-13C, and 14A-14G provide GenBank accession numbers. By accessing the sites indicated by the accession numbers, one in the art can obtain the nucleotide sequence and polypeptide sequence for the listed gene. In some embodiments, the array has 10 or more nucleic acids. In other embodiments, the array has 100 or more nucleic acids. In yet other embodiments, the array has not more than 100 nucleic acids, or not more than 300 nucleic acids. In certain embodiments of the invention, the sequence is 30 or more nucleotides in length. The reference nucleic acid and the test nucleic acid can be cDNAs, that are, in some embodiments, fluorescently labeled.

The invention includes a nucleic acid array having 4 or more nucleic acids immobilized on a solid support, each nucleic acid having a sequence of 10 or more consecutive nucleotides within any one of sequences listed in FIGS. 1, 2A-2R, 3A-3E, 6A-6E, 7A-7U, 8A-8I, 9, 13A-13C, and 14A-14G. In some embodiments, the array has 100 or more nucleic acids. In other embodiments, the array has not more than 100 nucleic acids, not more than 200 nucleic acids, or not more than 300 nucleic acids.

One aspect of the invention is an isolated nucleic acid molecule having a nucleotide sequence from any one of SEQ ID NOS: 1-3, or a complement thereof. In some embodiments of the invention, the isolated nucleic acid sequence has a non-nucleic acid modifying group bound to either a 3′ or 5′ end of the nucleotide sequence or both; or a synthetic nucleic acid sequence bound to a 3′ or 5′ end of the nucleic acid sequence or both.

The invention also includes an isolated polypeptide having an amino acid sequence encoded by a nucleic acid sequence from any one of SEQ ID NOS: 1-3.

Another embodiment of the invention is an isolated nucleic acid molecule having a nucleic acid sequence from any one of SEQ ID NOS: 4-93, or a complement thereof. In certain embodiments, the nucleotide sequence has a non-nucleic acid modifying group bound to either a 3′ or 5′ end of the nucleotide sequence or both; or a synthetic nucleic acid sequence bound to a 3′ or 5′ end of the nucleic acid sequence or both. The invention includes an isolated nucleic acid molecule having a nucleic acid sequence selected from SEQ ID NOS: 4-93, or a complement thereof. The invention also includes an isolated polypeptide having an amino acid sequence encoded by a nucleic acid sequence selected from any one of SEQ ID NOS: 4-93.

In one aspect, the invention is method for identifying a candidate agent, that modulates the expression or activity of a glucose transport-related polypeptide. The method includes the steps of providing a sample containing a glucose transport-related polypeptide; adding a test agent to the sample; assaying the sample for expression or activity of the glucose transport-related polypeptide; and comparing the effect of the test agent on expression or activity of the glucose transport-related polypeptide relative to a control. A change in glucose transport-related polypeptide expression or activity indicates that the test agent is a candidate agent that can modulate expression or activity of the glucose transport-related polypeptide. In some aspects of the method the test agent is a polynucleotide, a polypeptide, a small non-nucleic acid organic molecule, a small inorganic molecule, an antibody, an antisense oligonucleotide, or a ribozyme. In yet another embodiment, the glucose transport-related polypeptide is assayed using an antibody. In some embodiments of the invention, the glucose transport-related polypeptide is a human glucose transport-related polypeptide. The method can include the additional step of determining whether glucose transport is modulated in the presence of the test agent. The test agent can decrease or increase glucose transport. The assay can be a cell based assay or a cell-free assay. In certain embodiments of the invention, the glucose transport-related polypeptide is selected from the group of polypeptides encoded by sequences having the nucleic acid sequences listed in FIGS. 1, 2A-2R, and 3A-3E, and the polypeptides listed in FIGS. 6A-6E, 7A-7U, 8A-8I, 9, 13A-13C, and 14A-14G 6-9.

Modulation of expression (nucleic acid or polypeptide) or activity can be an increase or a decrease in expression or activity compared to a reference. The amount of modulation is generally at least two fold (i.e., a two fold increase or decrease in expression or activity) compared to a reference or a control sample. For example, the amount of modulation can be five fold, ten fold, fifty fold, 100 fold, or more.

The invention includes a method for identifying a candidate agent that modulates expression of a glucose transport-related polynucleotide. The method includes the steps of providing a sample in which a glucose transport-related polynucleotide is expressed; adding a test agent to the sample; detecting expression of the glucose transport-related polynucleotide; determining the amount of expression of the glucose transport-related polynucleotide; and comparing the effect of the test agent on the amount of expression of the glucose transport-related polynucleotide in the sample relative to a control, such that a change in the amount of expression from the glucose transport-related polynucleotide indicates the test agent is a candidate agent that can modulate expression of the glucose transport-related polynucleotide. The test agent can be a polynucleotide, a polypeptide, a small non-nucleic acid organic molecule, a small inorganic molecule, an antibody, an antisense oligonucleotide or a ribozyme. In some embodiments, the glucose transport-related polynucleotide is a human glucose transport-related polynucleotide. In another aspect of the invention, the method includes the step of determining whether glucose transport is modulated (e.g., increased or decreased) in the presence of the test agent. In some embodiments, the glucose transport-related polynucleotide is selected from the group of sequences listed in FIGS. 1, 2A-2R, and 3A-3E-3 or a complement thereof, and listed in FIGS. 6A-6E, 7A-7U, 8A-81, 9, 13A-13C, and 14A-14G, or a complement thereof. The assay used in the method can be cell-based assay or a cell-free assay.

The invention includes a method of diagnosing an individual having or at risk for a glucose transport-related disorder. The method includes the steps of providing a nucleic acid array having 4 or more nucleic acids immobilized on a solid support, each nucleic acid having a sequence of 10 or more nucleotides, the sequence having or containing a sequence selected from the group of the sequences listed in FIGS. 1, 2A-2R, and 3A-3E, or a complement thereof, and the sequences of the genes listed in FIGS. FIGS. 6A-6E, 7A-7U, 8A-8I, 9, 13A-13C, and 14A-14G, or a complement thereof; providing a nucleic acid sample from the individual; contacting the array with the sample from the individual; detecting hybridization of nucleic acid in the sample from the individual with each nucleic acid in the array, to obtain a pattern of glucose transport-related gene expression; comparing the pattern of glucose transport-related gene expression in sample from the individual with a reference pattern, such that a comparison of the pattern of expression in the individual compared to the reference pattern indicates whether the individual has or is at risk for a glucose transport-related disorder. In some aspects of the invention, the array has 10 or more nucleic acids; or 100 or more nucleic acids. In other aspects of the invention, the array has not more than 100 nucleic acids; not more than 200 nucleic acids, or not more than 300 nucleic acids. In some embodiments, the sequence has 30 or more nucleotides. The sample from the individual can be a cDNA sample, and the cDNA sample can be fluorescently labeled. In some embodiments, the disorder is type II diabetes.

The invention also includes a nucleic acid array having 4 or more nucleic acids immobilized on a solid support, each nucleic acid comprising a sequence of 10 or more nucleotides, the sequence consisting of at least a portion of a sequence selected from the sequences listed in FIGS. 1, 2A-2R, and 3A-3E, or a complement thereof, FIGS. 6A-6E, 7A-7U, 8A-8I, 9, 13A-13C, and 14A-14G, or a complement thereof.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference. In addition, the materials, methods, and examples are illustrative only and not intended to be limiting.

Other features and advantages of the invention will be apparent from the detailed description, and from the claims.

### DESCRIPTION OF DRAWINGS

FIG. 1 is a depiction of nucleic acid sequences identified in the Muscle Adipocyte Union library; c0148 (SEQ ID NO: 1), c0827 (SEQ ID NO: 2), and c1083 (SEQ ID NO: 3).

FIGS. 2A-2R are a series of sequences identified in the Muscle-Adipocyte Union Library (MAU library) that contain previously unidentified sequences and ESTs.

FIGS. 3A-3E are series of sequences identified in the Adipocyte Subtractive (subtractive) library that contain previously unidentified sequences and ESTs.

FIG. 4 is a diagram showing a suppression subtractive hybridization protocol.

FIG. 5 is a diagram showing a protocol for constructing the Muscle-Adipocyte Union library.

FIGS. 6A-6E are a table showing genes expressed in the Adipocyte Subtractive Library.

FIGS. 7A-7U are a table showing genes expressed in the Muscle-Adipocyte Union Library.

FIGS. 8A-8I are a table showing the proteins identified in peaks 1 and 2 of GLUT4-associated vesicles.

FIG. 9 is a table listing those proteins/genes that are present in one or both of the subtractive and Muscle-Adipocyte-Union libraries and were also identified as proteins purified from Glut4 vesicles. “Yes” indicates that a peptide(s) corresponding to the protein was present in a preparation. “?” indicates that the protein has not yet been identified in this preparation but its presence has not been excluded.

FIGS. 10A-10D are a series of hydrophobicity plots of the c0582 sequence.

FIGS. 11A-11D are a series of hydrophobicity plots of the c0139 sequence.

FIGS. 12A-12D are a series of hydrophobicity plots of the b075 sequence.

FIGS. 13A-13C are a table listing genes whose expression was not detected in fibroblasts, and was detected in adipocyte or muscle using GeneChips. Columns marked f1 and f2 are data from the fibroblast replicate chips, columns marked a1 and a2 are data from the adipocyte replicate chips, and the columns marked m1 and m2 are data from the muscle replicate chips. A indicates that the gene is absent in a tissue. P indicates that the gene is present in a tissue. An M indicates marginal signal and the software cannot determine if the gene is absent or present.

FIGS. 14A-14G are tables listing genes whose expression was determined to be the same on all fibroblast chips, and increased on both adipocyte or muscle GeneChips compared to a fibroblast chip. The columns marked f1, f2, and f3 are fibroblast replicate chips. The columns marked a1, a2, and a3 are adipocyte replicate chips, and the columns marked m1, m2, and m3 are the muscle replicate chips. NC indicates no change of expression. MI indicates that there was a moderate increase in expression. An I indicates an increase in expression. The function classes of the genes listed in the last column are as follows: Class 1 genes encode metabolic proteins; Class 2 genes encode signaling proteins.

FIGS. 15A-15B are a table listing highly expressed genes common between the Muscle-Adipocyte Union library and the Mu-74 GeneChips Arrays.

### DETAILED DESCRIPTION

Library of Glucose Transport-Related Sequences

Suppressive subtraction hybridization has been applied to create libraries (databases) of glucose transport-related nucleotide sequences. The Muscle-Adipocyte Union library contains about 230 glucose transport-related nucleotide sequences and was made by identifying nucleotide sequences selectively expressed in fat and muscle tissue, but not in fibroblasts. Sequences from the subtractive library or the MAU library can be used in the invention. Generally, the sequences are from the MAU library. Unless indicated otherwise below, the library referred to is the MAU library. The sequences in the library represent glucose transport-related genes that are candidates for involvement in insulin-related action, and thus potential drug targets for glucose transport-related disorders. Glucose transport-related disorders include diseases such as type II diabetes, obesity, certain types of cardiovascular disease, and Syndrome X.

The library can be used to construct DNA arrays for identifying glucose transport-related genes whose expression is altered (increased or decreased) in diseases or disorders characterized by insulin resistance, e.g., type II diabetes, or defects in glucose transport. The library advantageously enables gene expression pattern comparisons that involve tens or hundreds of genes most likely to be involved in insulin resistance and type II diabetes, instead of comparisons that involve tens of thousands or hundreds of thousands of genes. This focus on a relatively small library advantageously simplifies data analysis and improves the signal-to-noise ratio. In addition to being useful for identifying individual glucose transport-related genes, DNA arrays of the invention can be used to identify gene expression patterns indicative of particular forms of type II diabetes or a predisposition (i.e., at risk for) for development of type TI diabetes. The predisposition can be a genetic predisposition.

Once specific glucose transport-related genes are identified using the library, assays for expression of individual genes can be employed. Specific assays can be employed, for example, in diagnostic methods to diagnose type II diabetes, methods for diagnosing particular forms of type II diabetes, and methods for identifying individuals who have pre-symptomatic forms of type II diabetes or a genetic predisposition for development of type II diabetes. Such diagnostic assays may provide useful information for devising therapeutic strategies tailored to individual patients.

The library can also be used to assay expression of individual genes in animal (e.g., mouse) models of a disease in which glucose transport is affected. For example, cDNA can be prepared from RNA isolated from a mouse having a glucose transport-related disorder such as diabetes. The RNA can be isolated from a tissue that normally carries out glucose transport (e.g., muscle or adipose tissue). The cDNA is hybridized to sequences from the MAU library. Expression of the MAU library sequences is then compared to expression of the sequences in a mouse that does not have the disorder. A relative increase or decrease in the expression of a sequence in the mouse having a glucose transport disorder compared to an unaffected mouse indicates that the sequence is involved in the disorder. Such sequences are useful, e.g., for indicating genes or gene products as drug targets for treating the disorder.

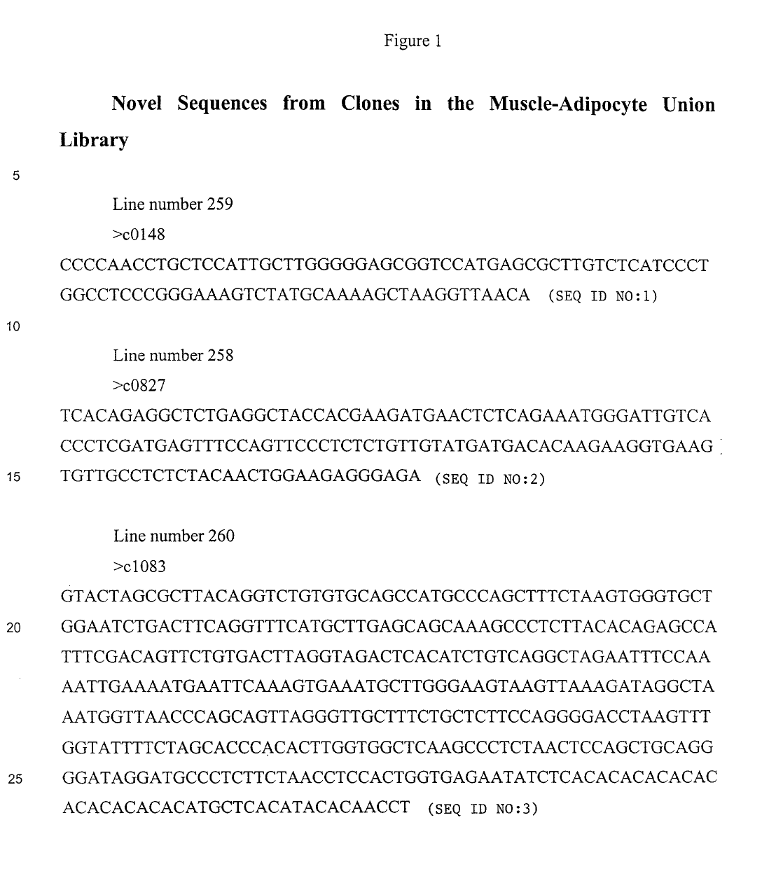

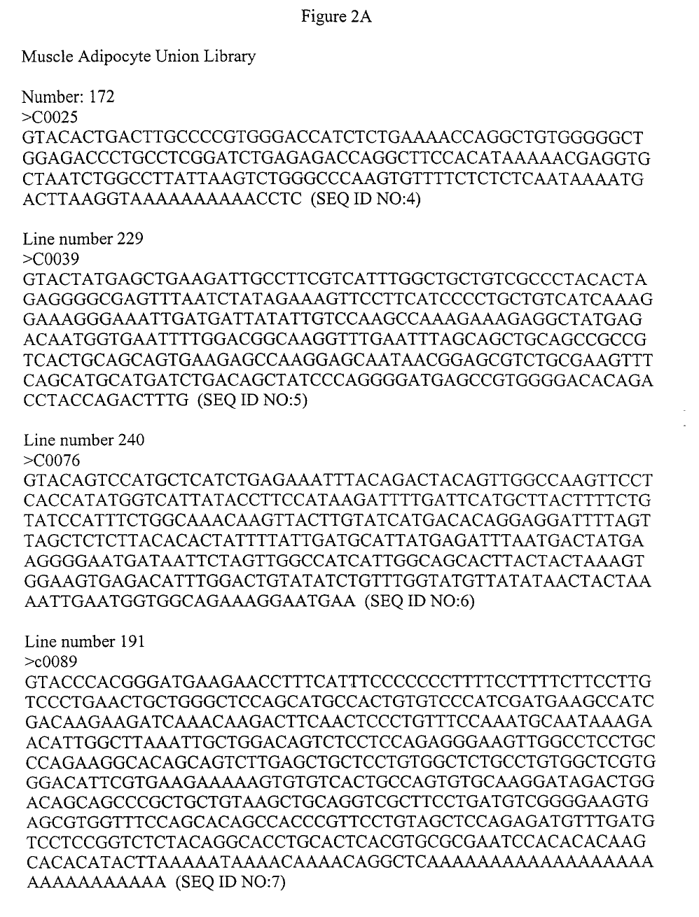

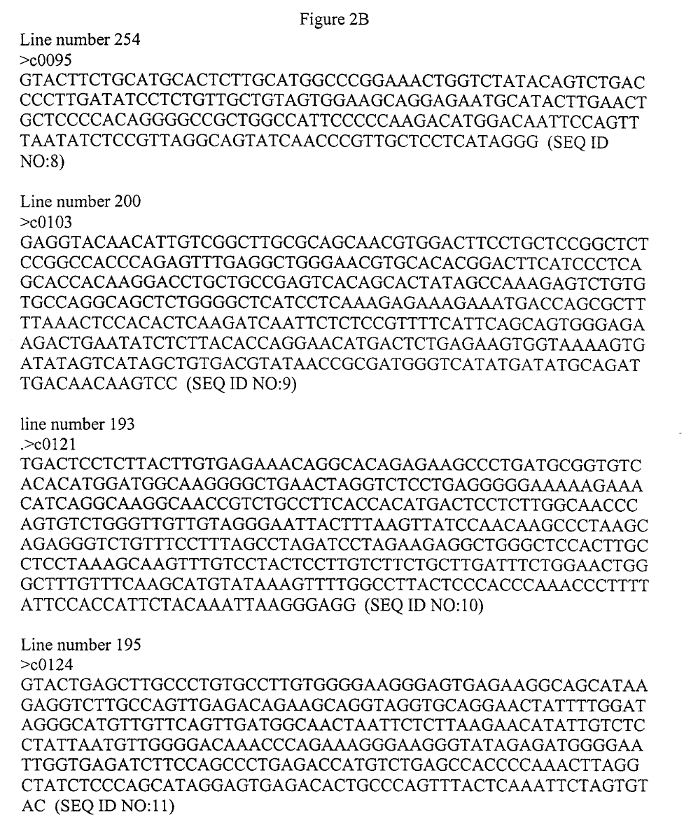

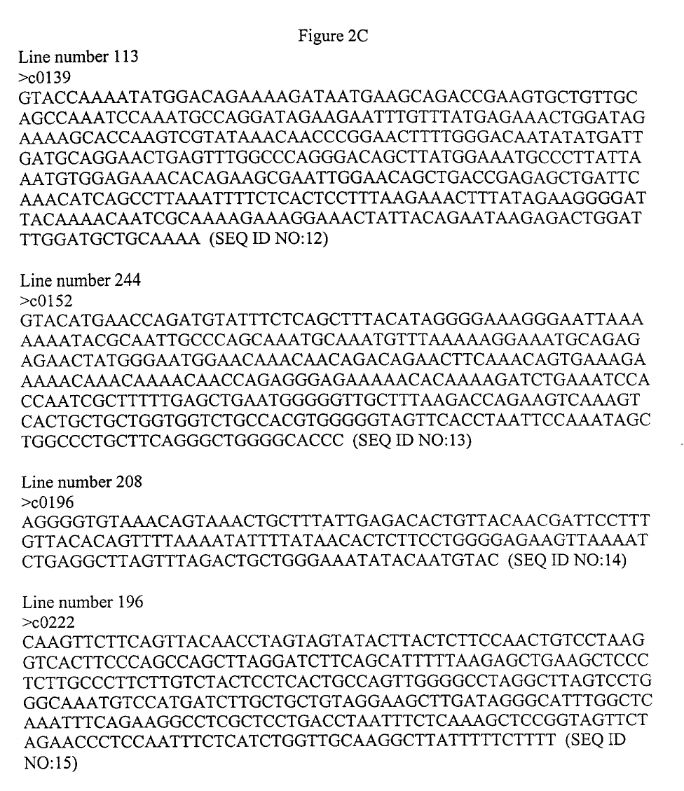

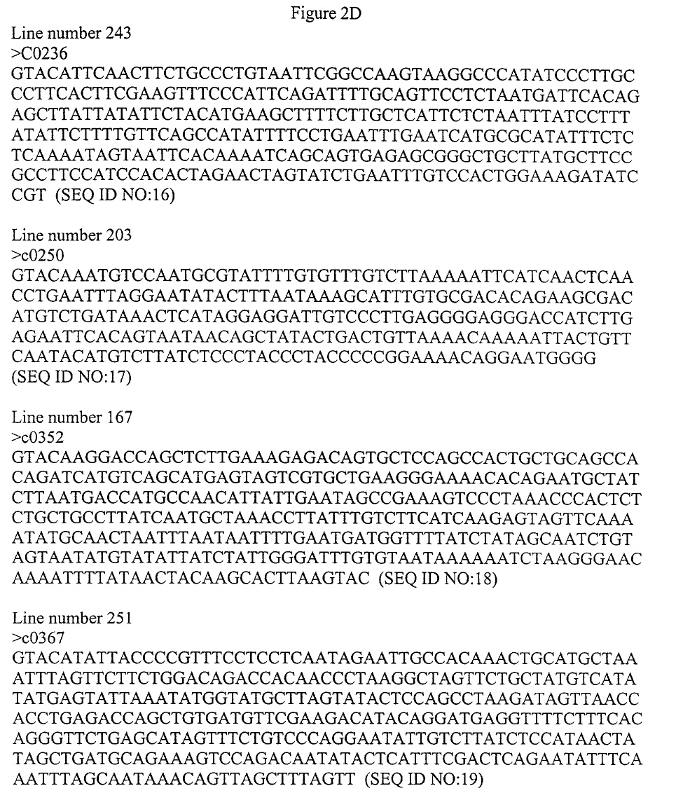

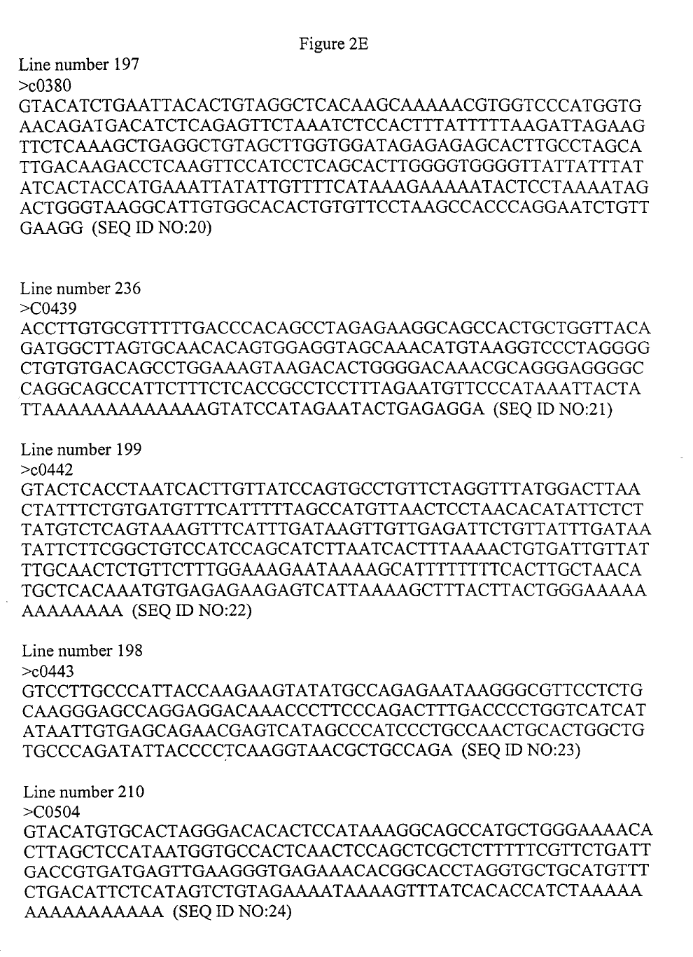

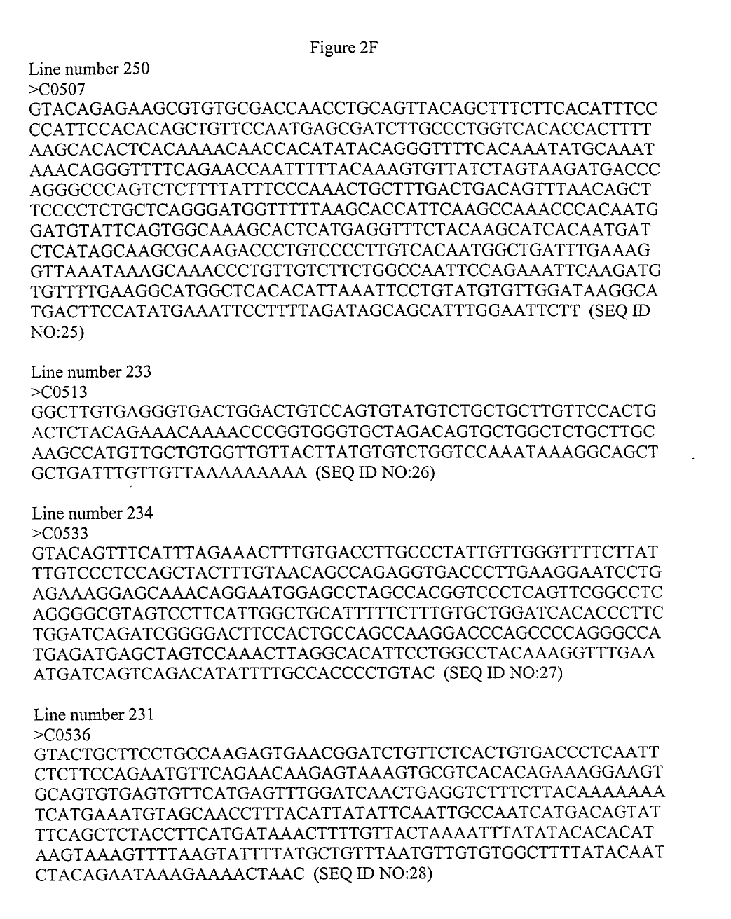

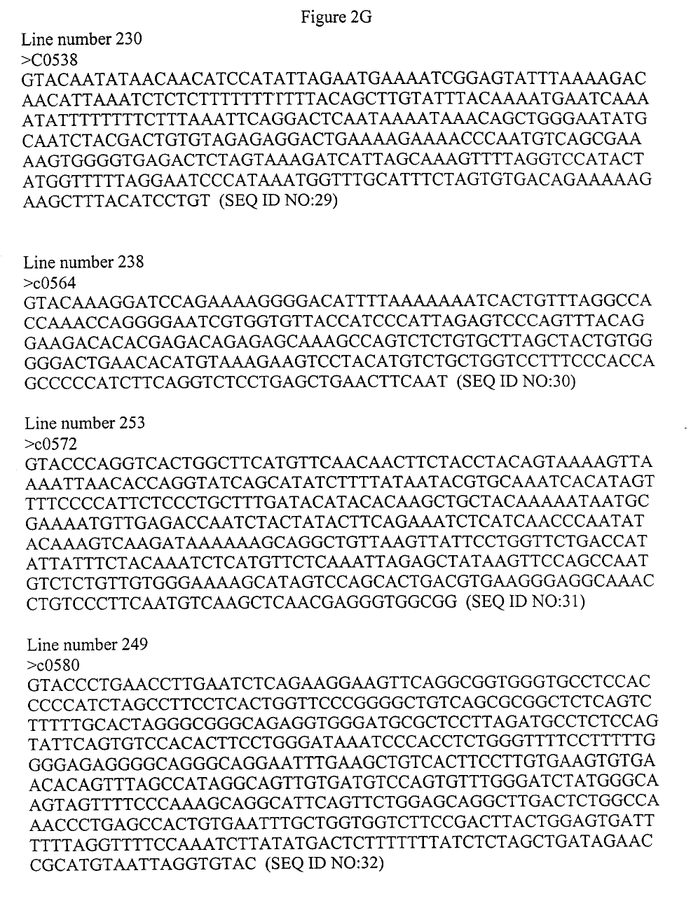

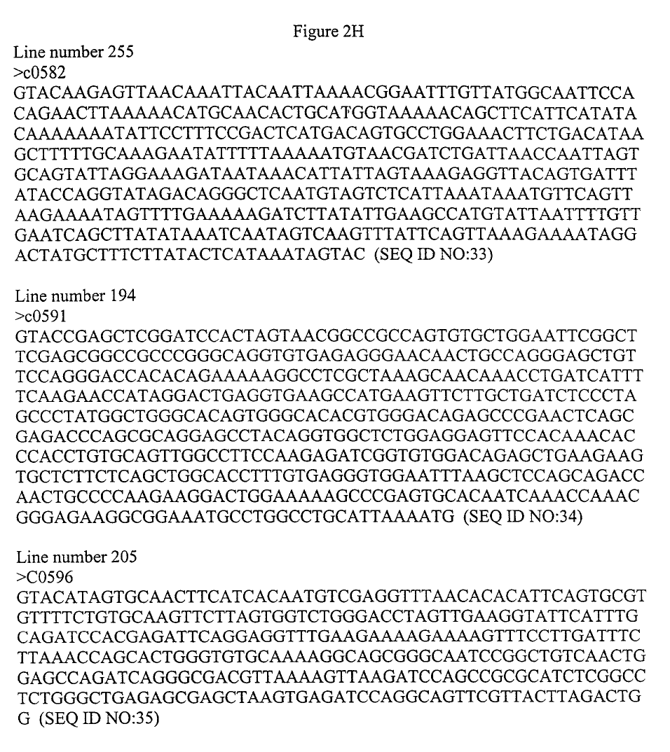

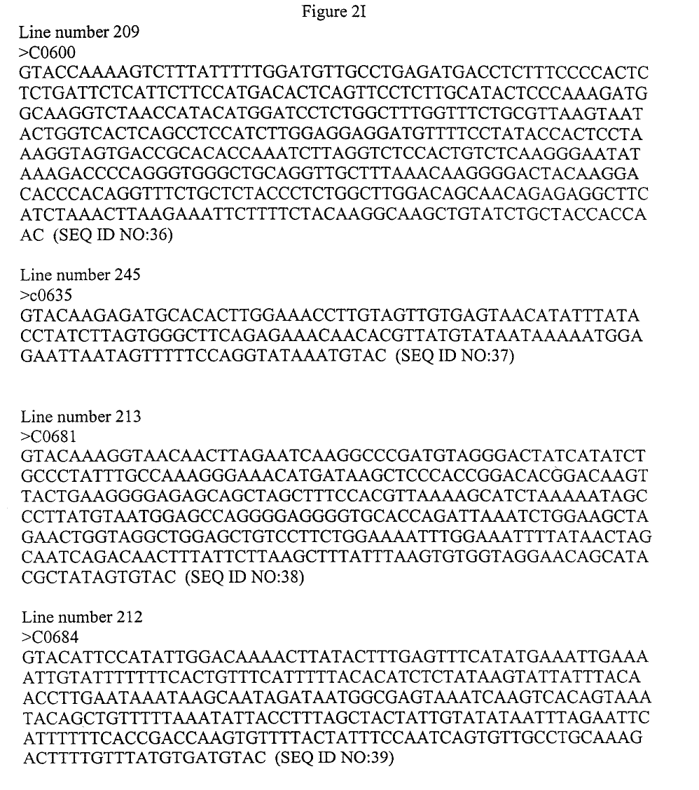

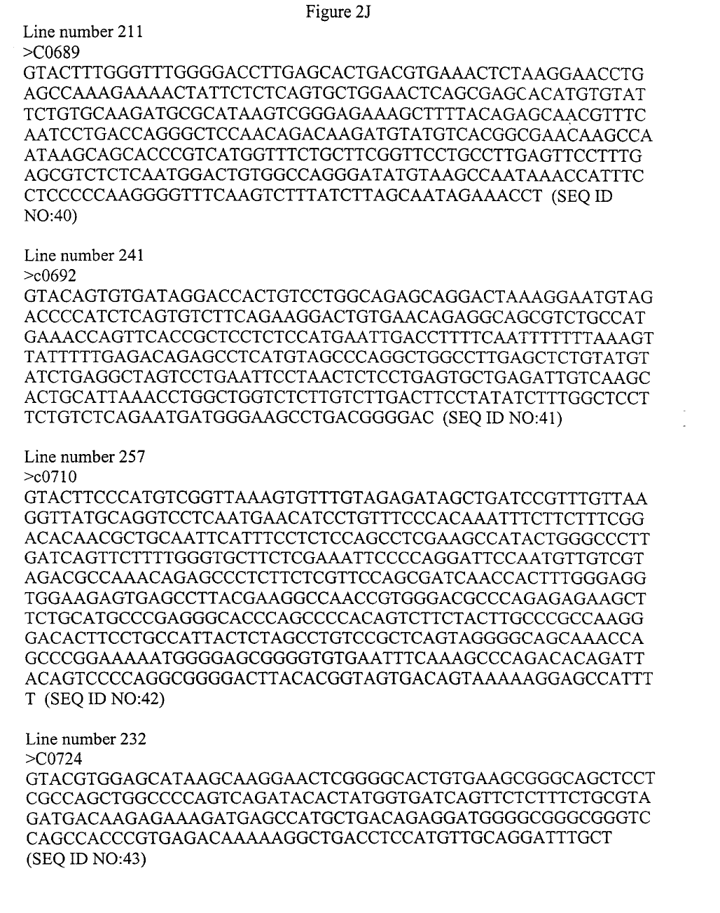

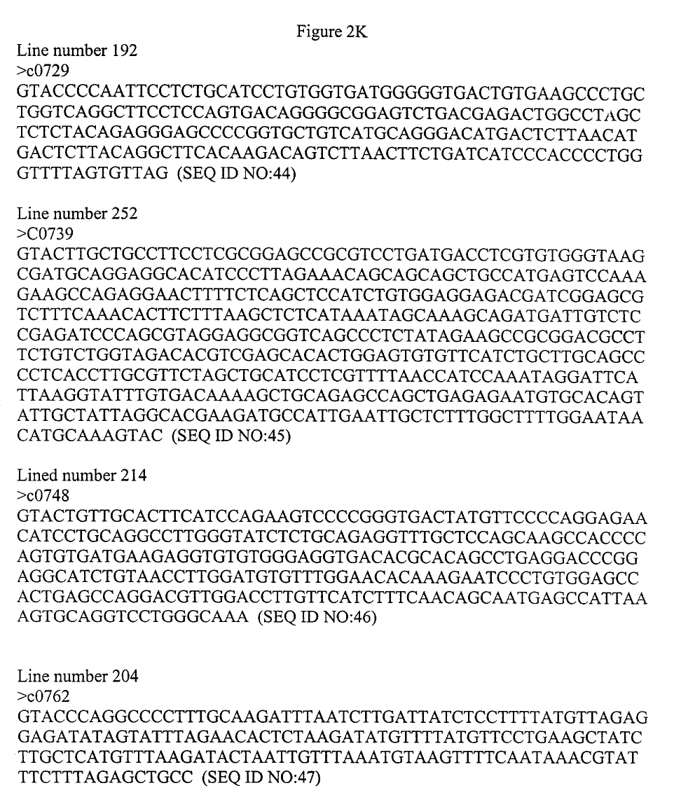

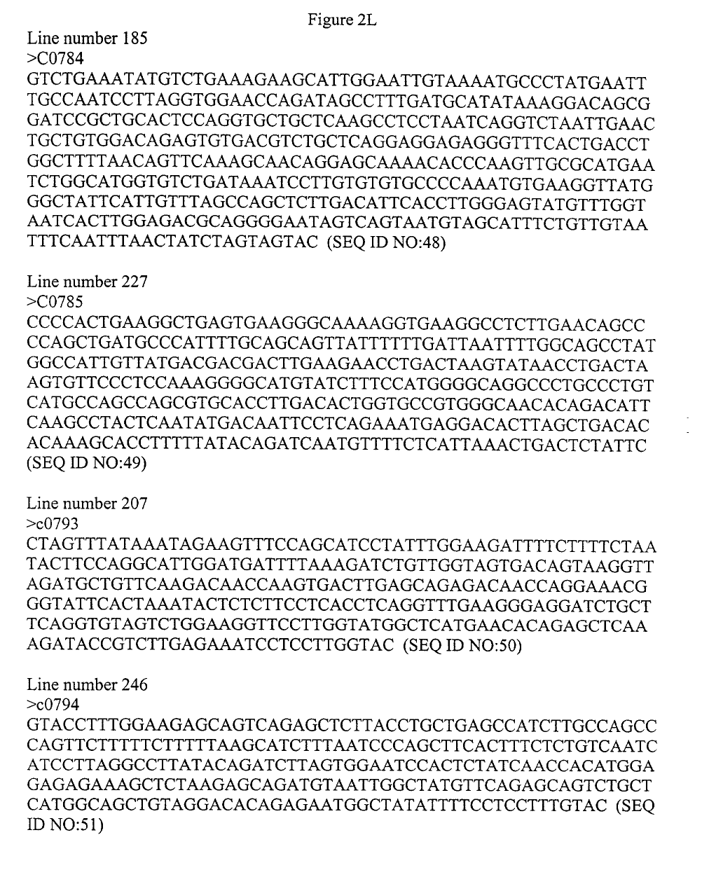

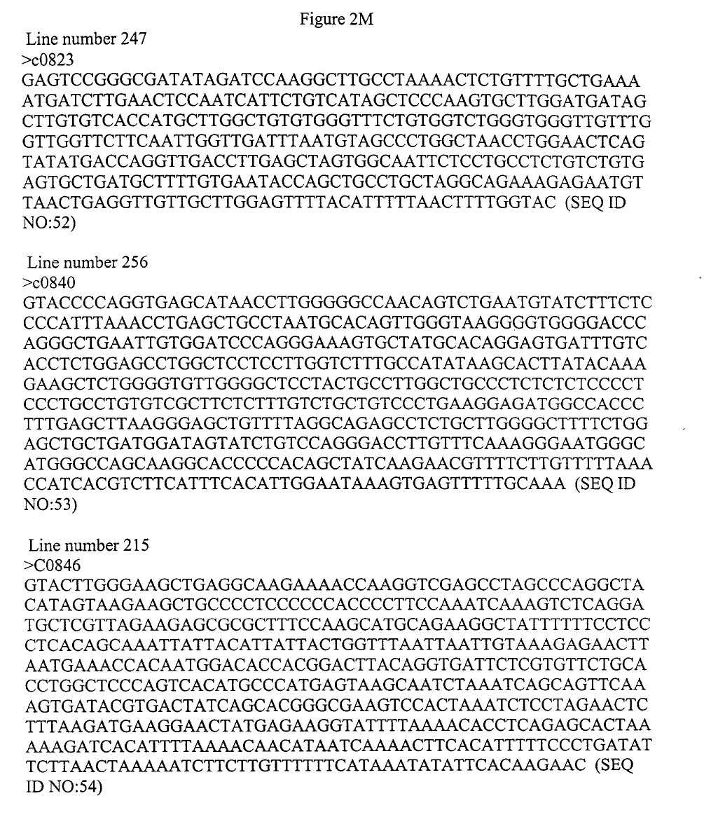

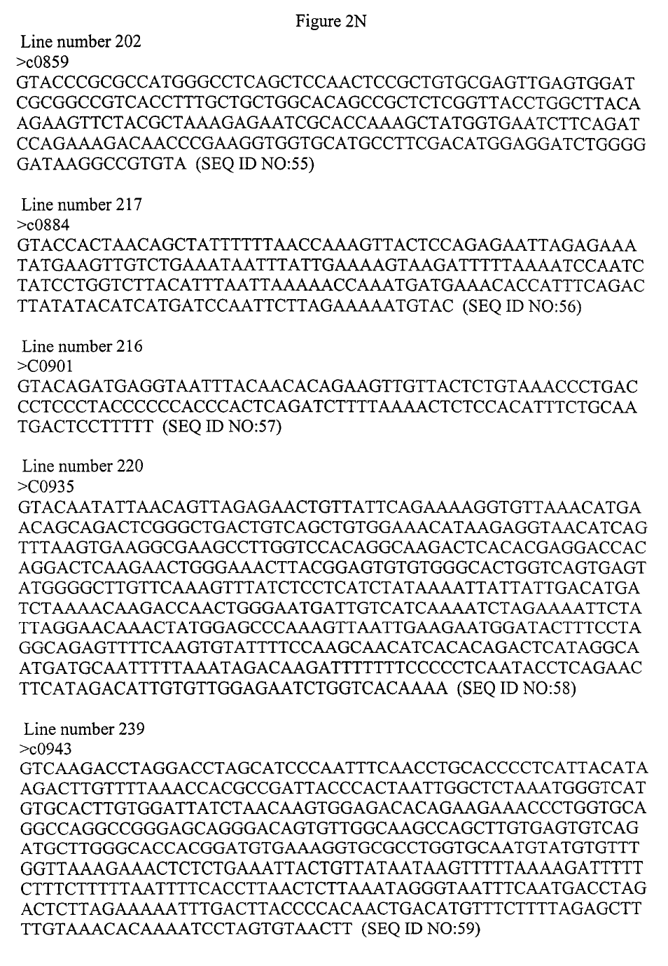

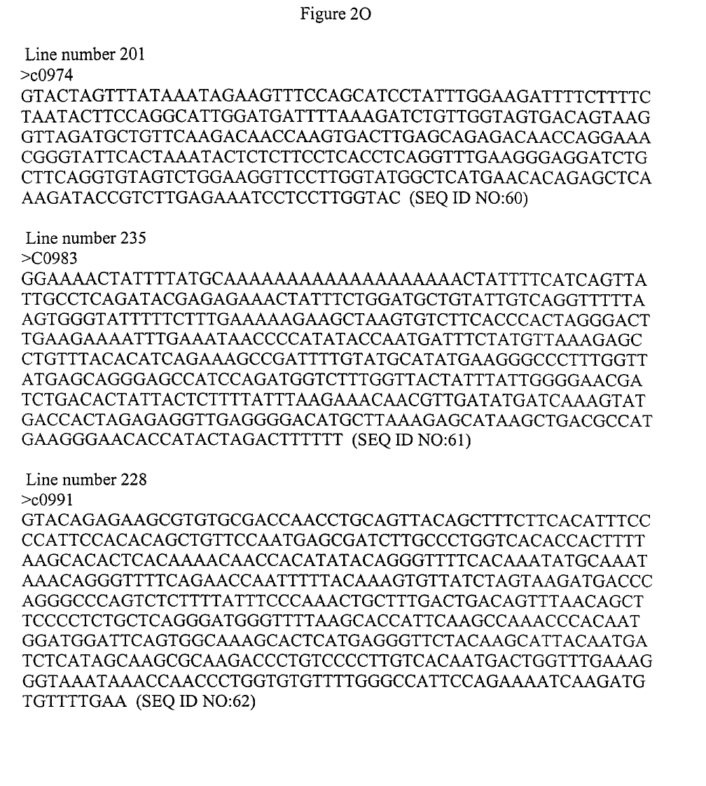

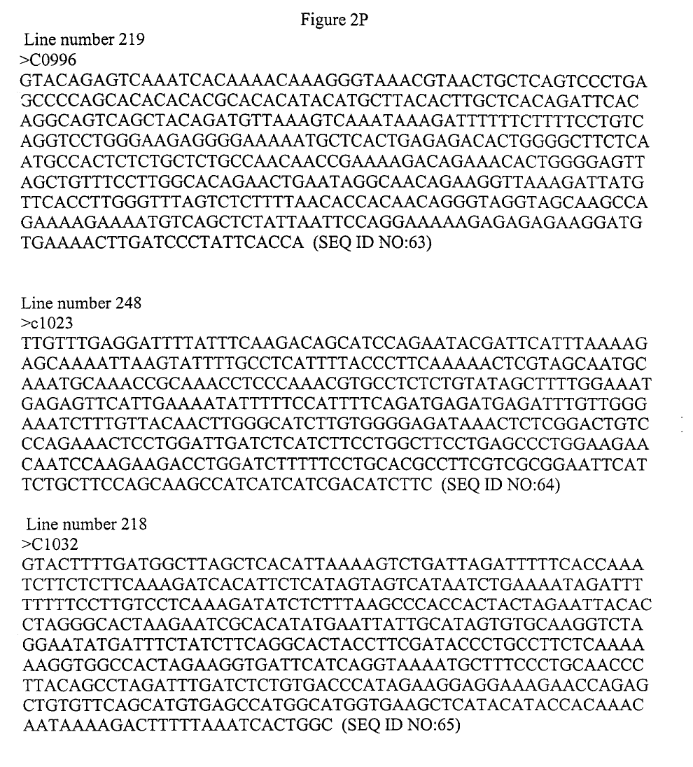

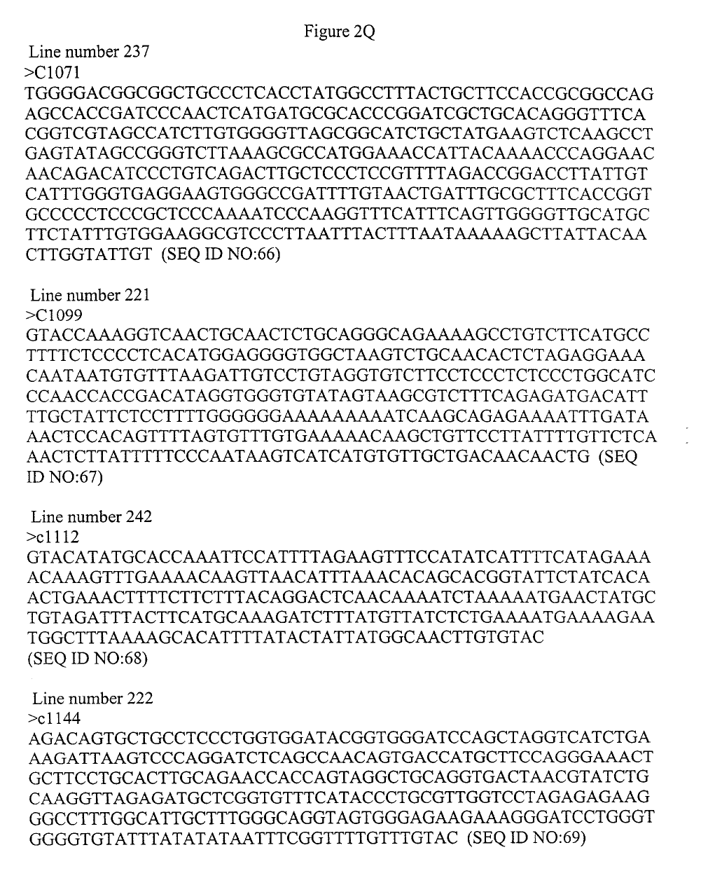

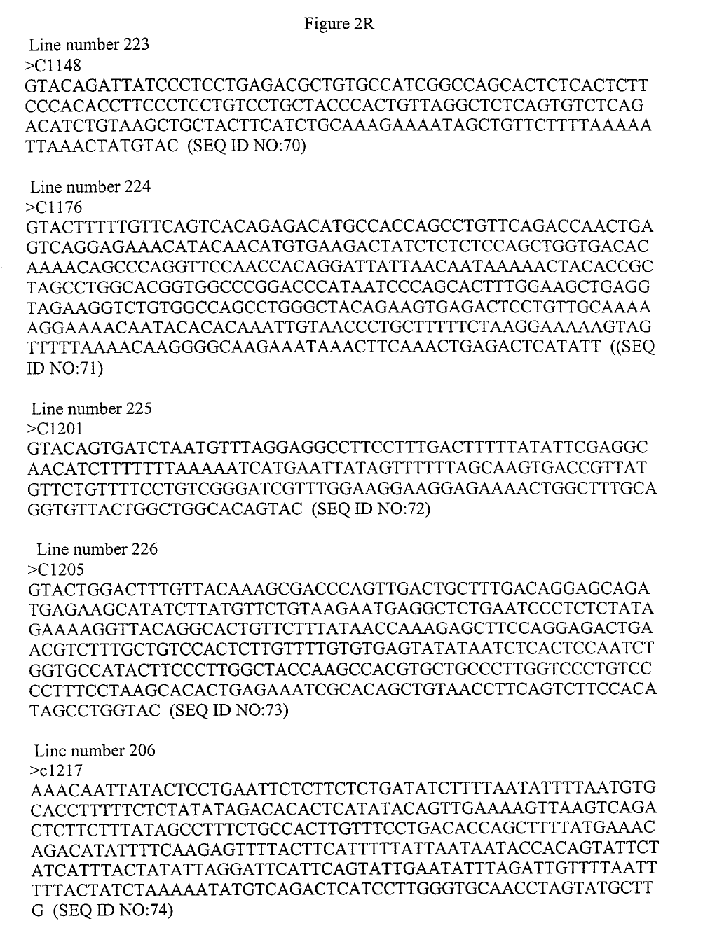

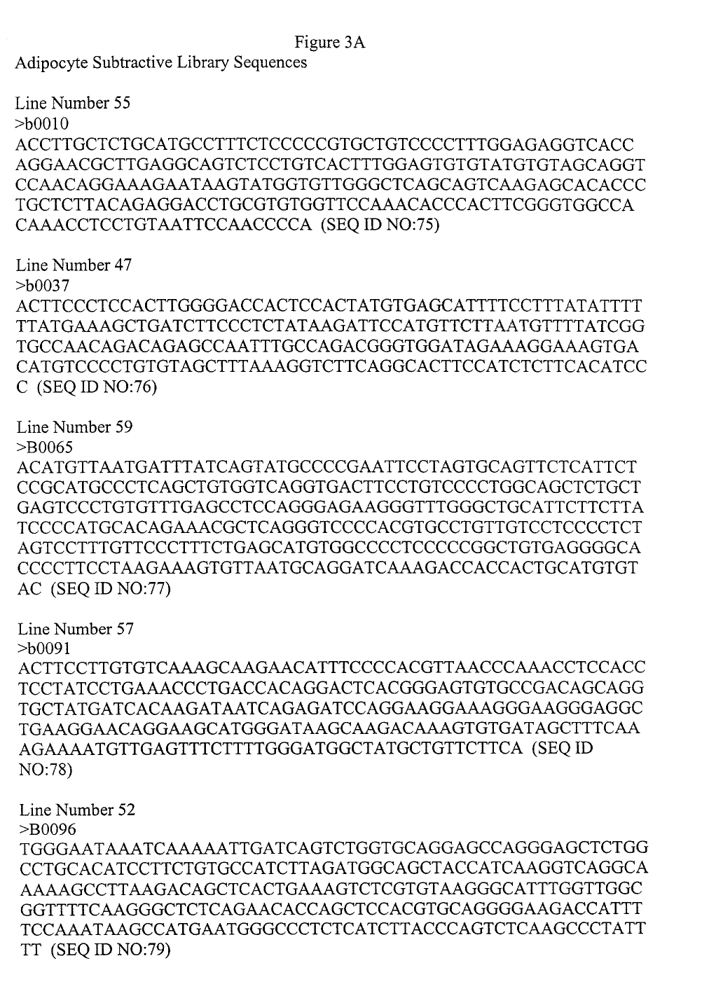

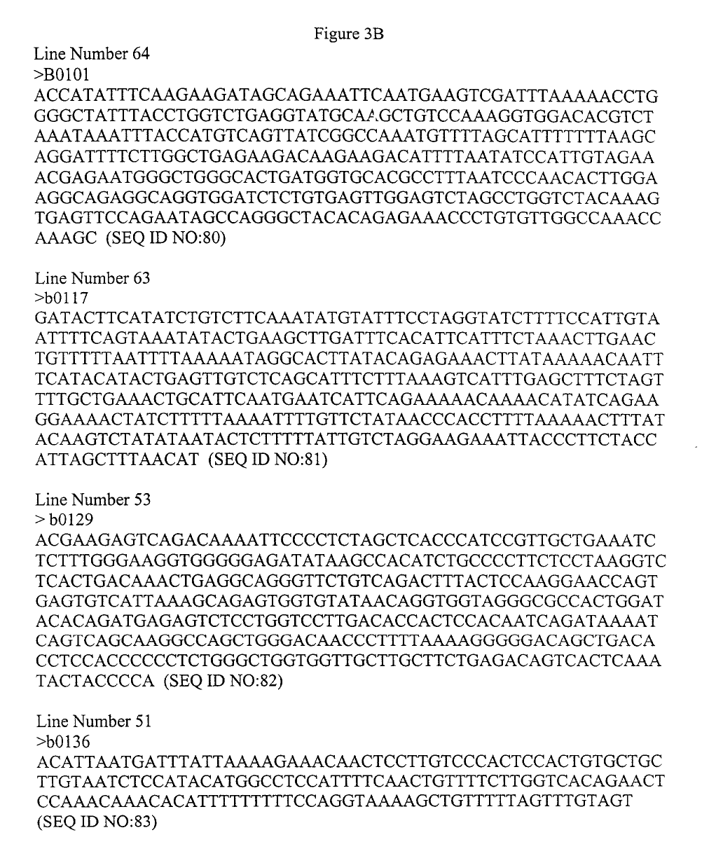

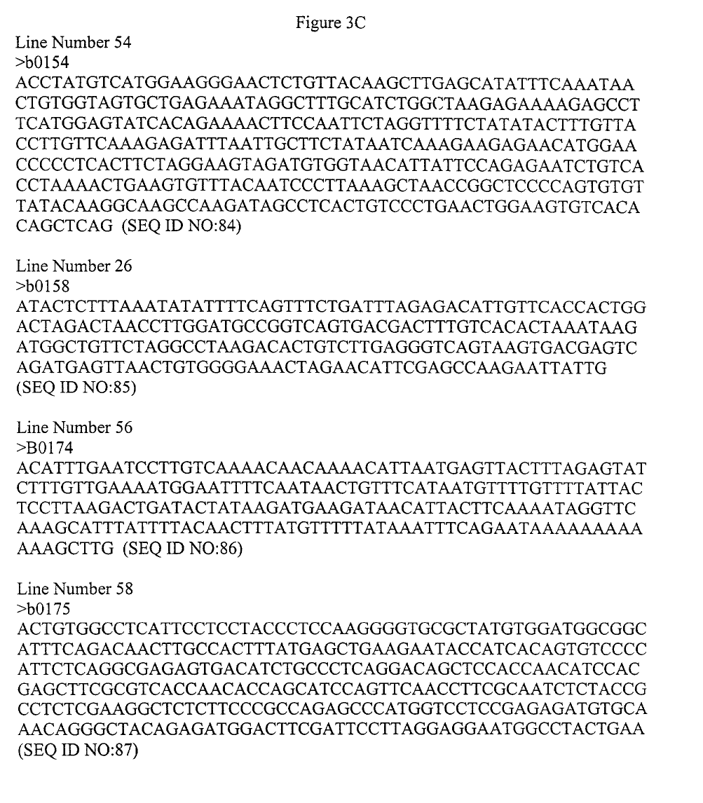

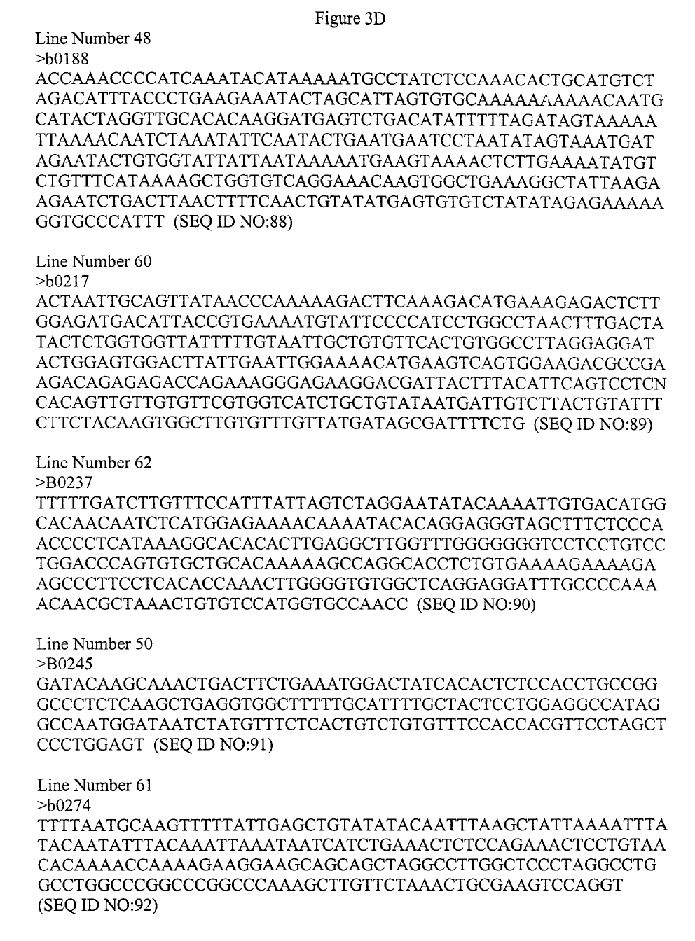

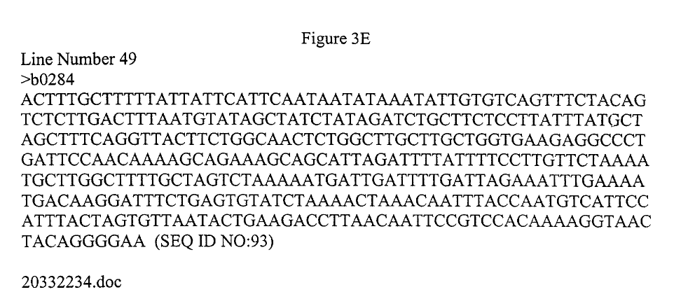

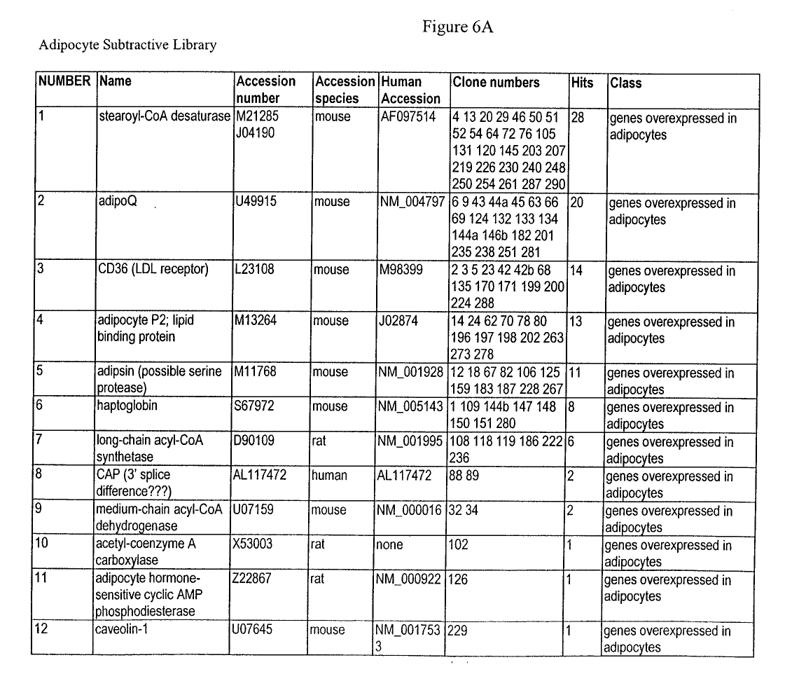

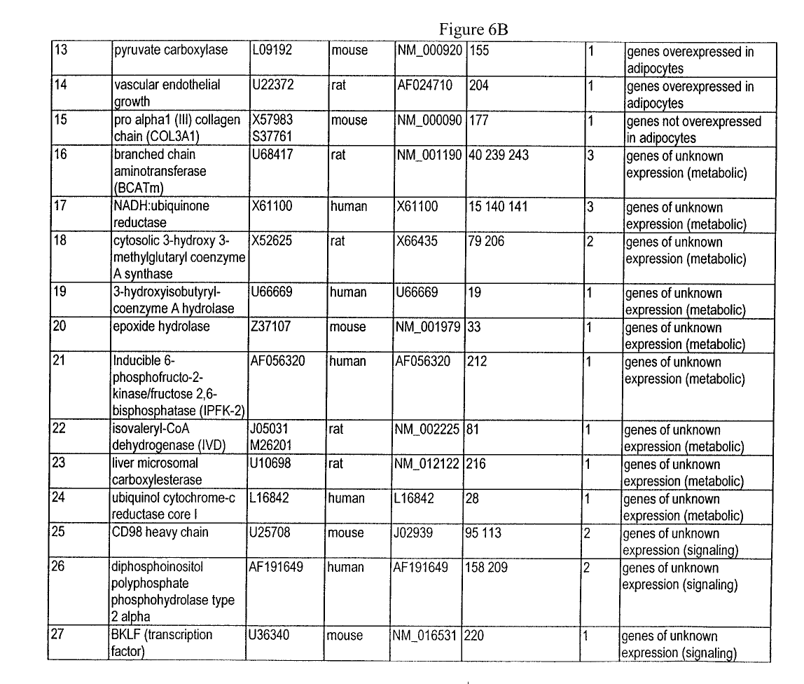

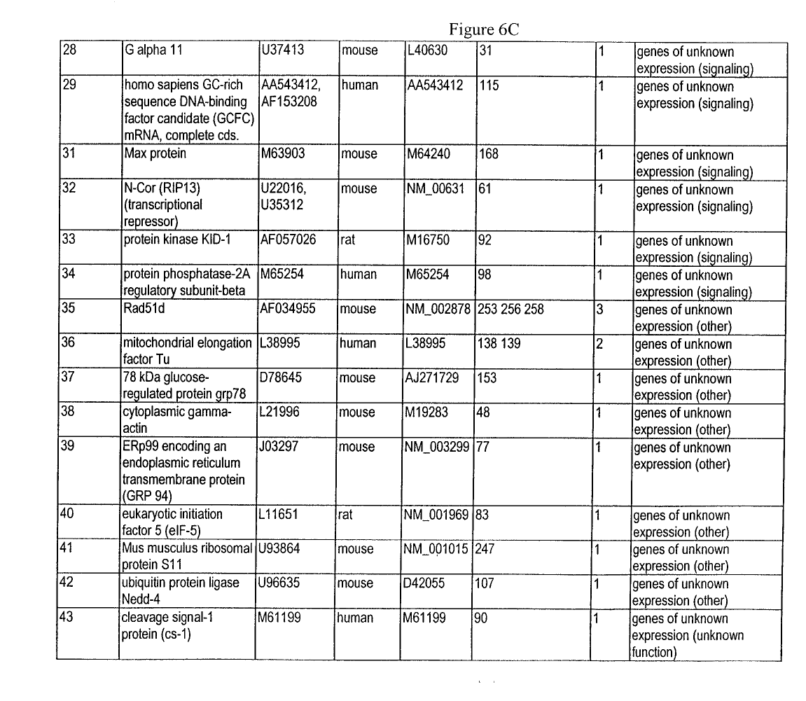

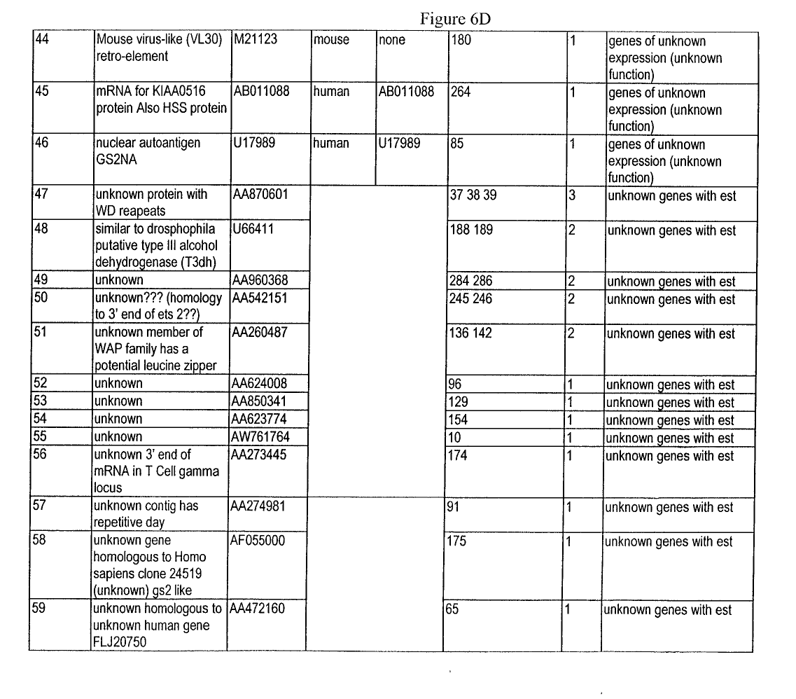

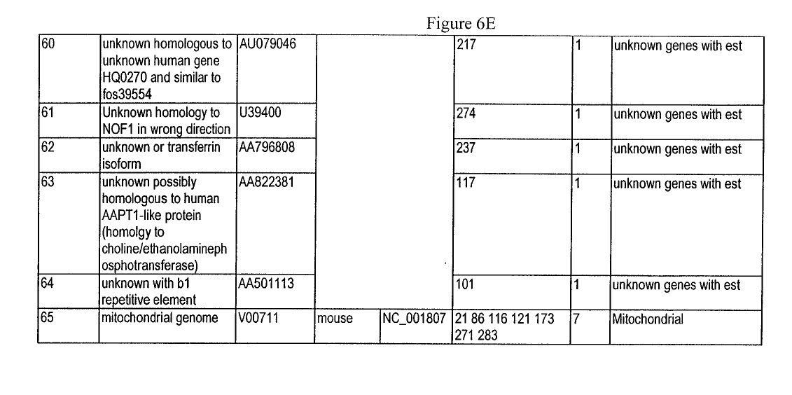

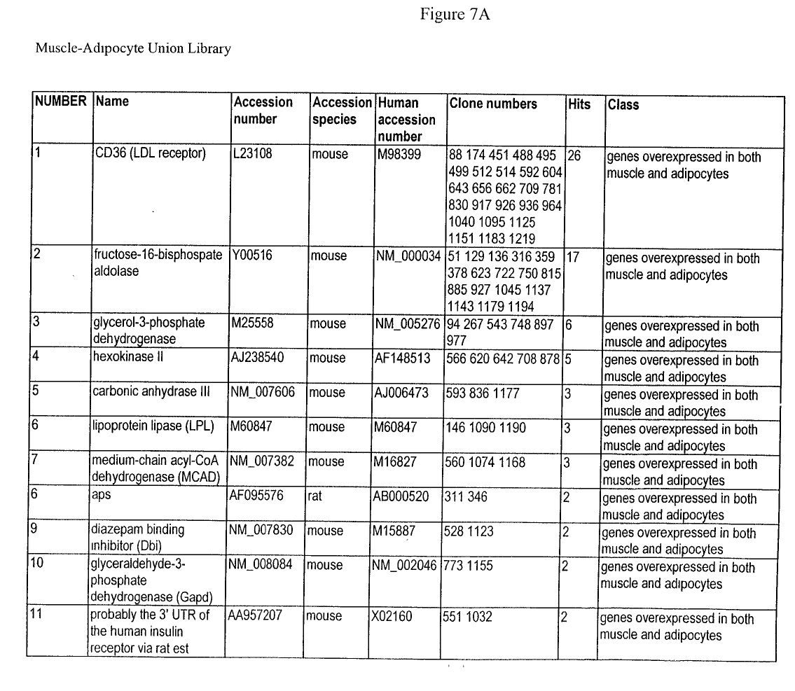

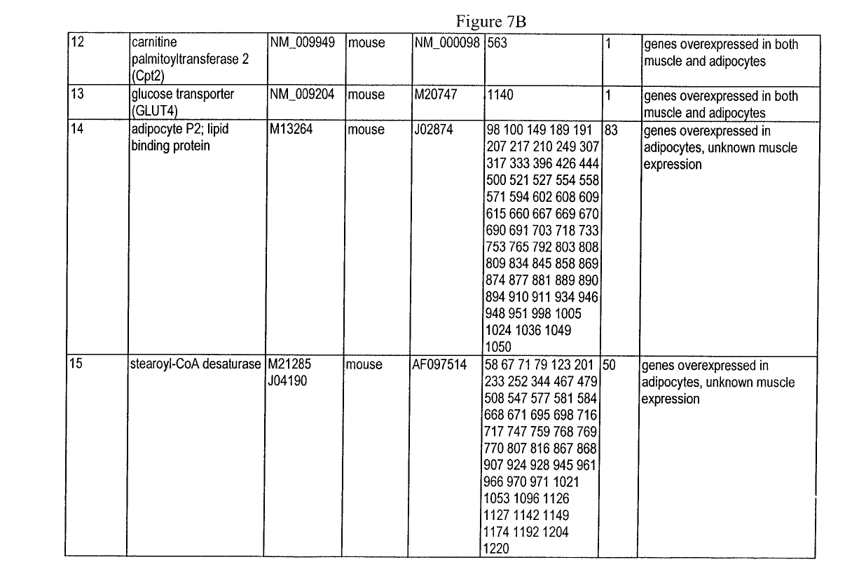

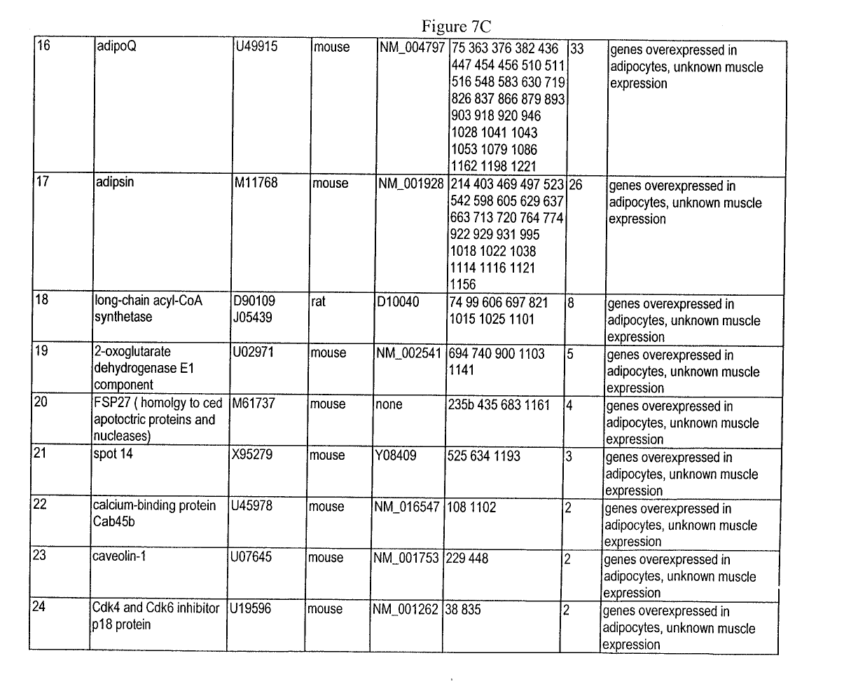

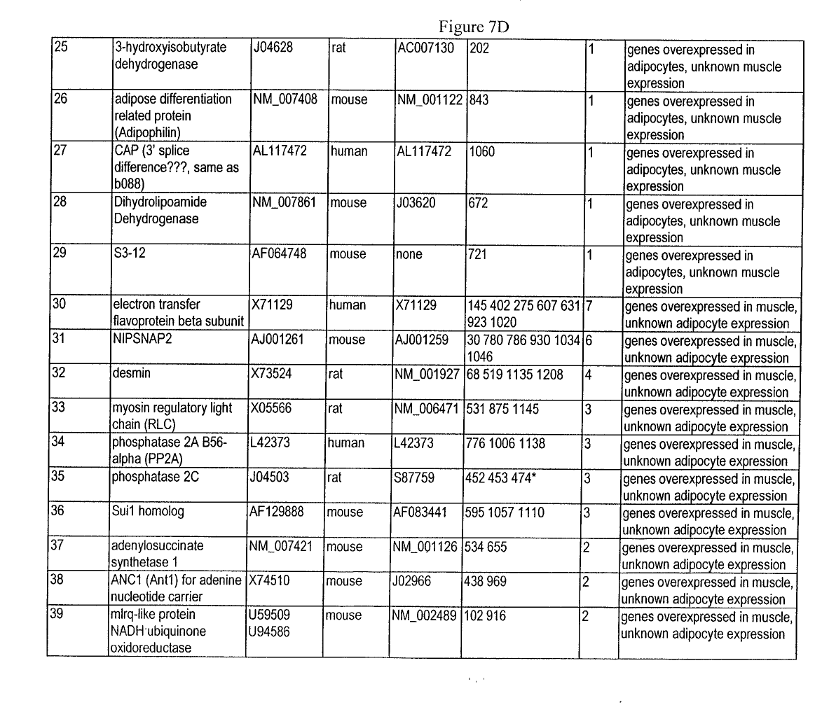

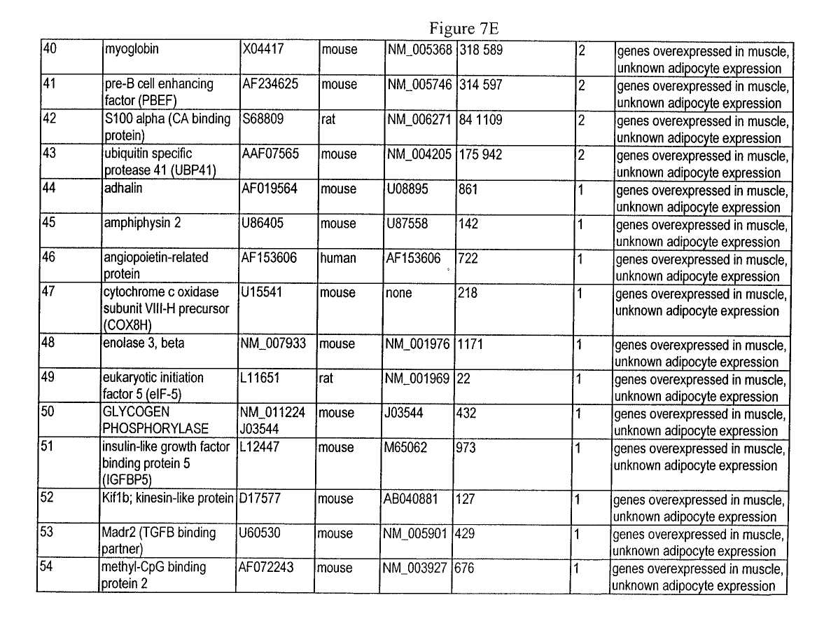

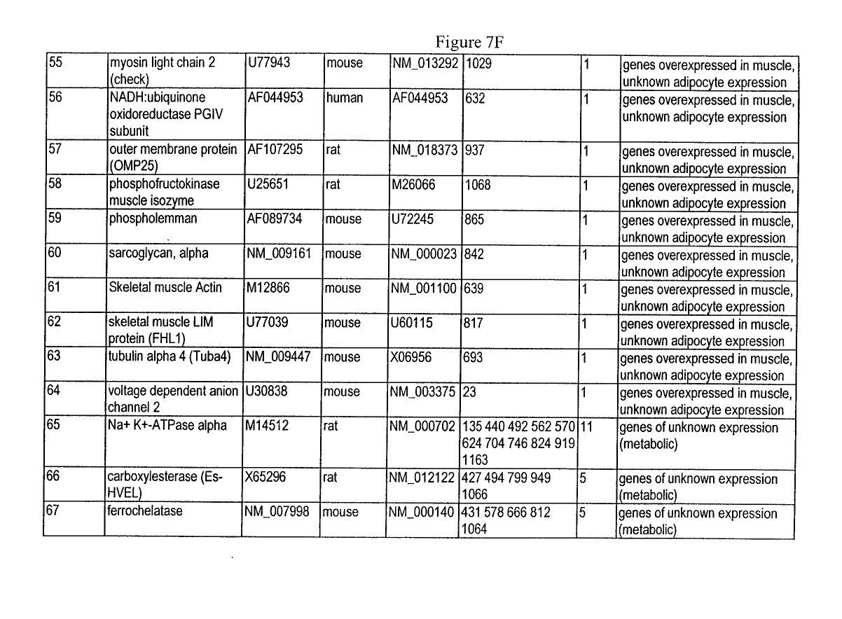

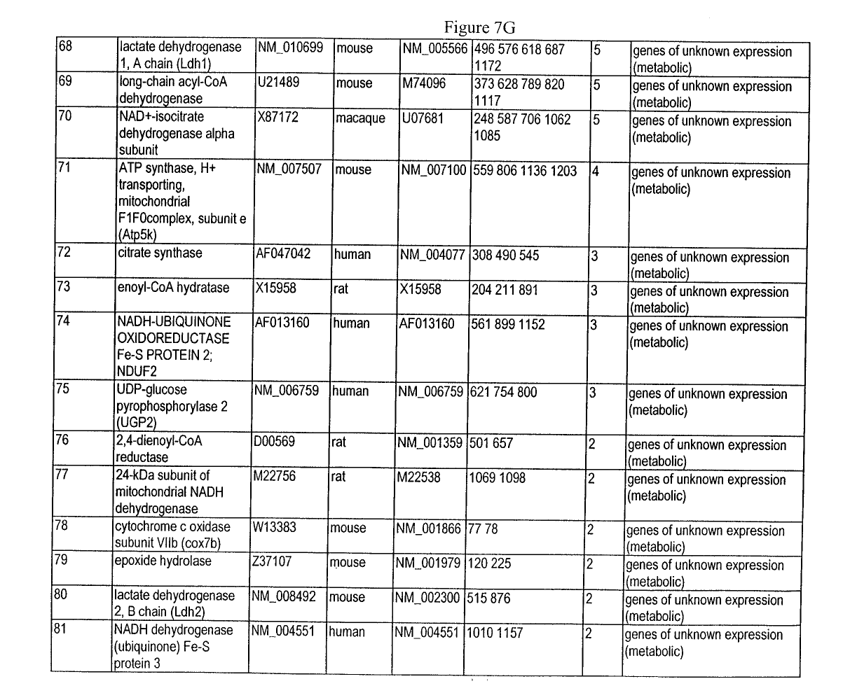

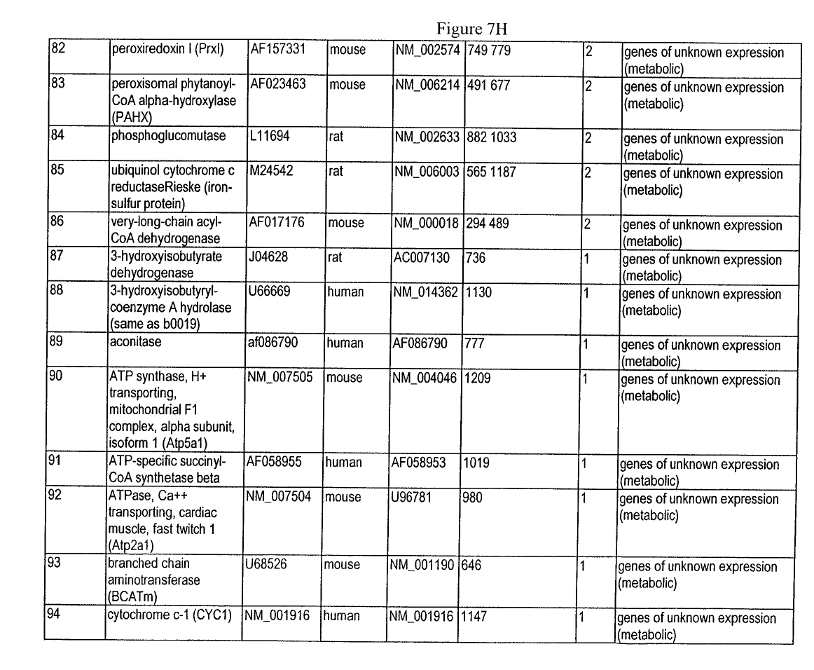

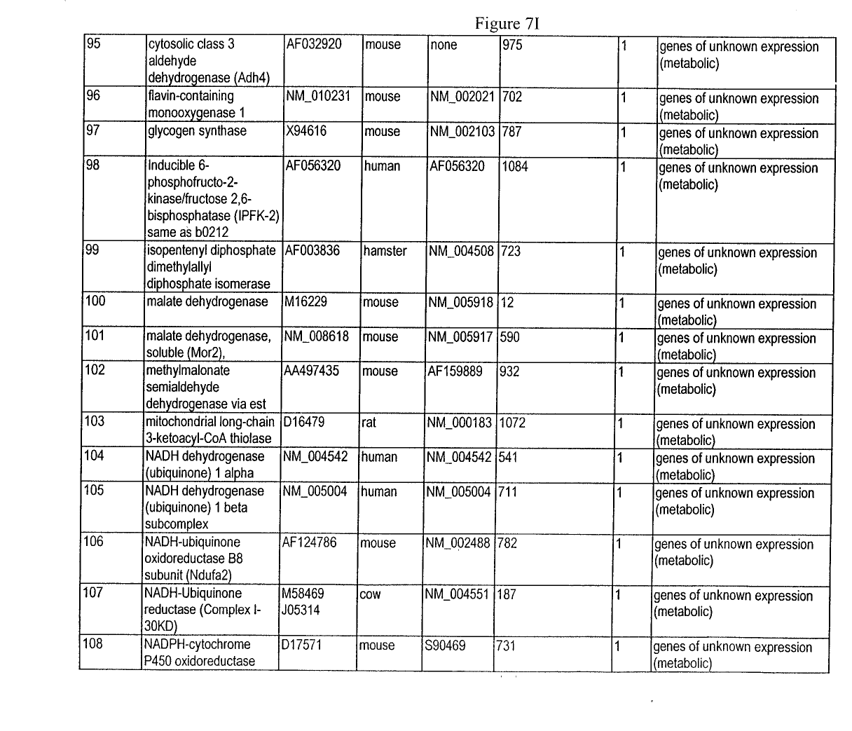

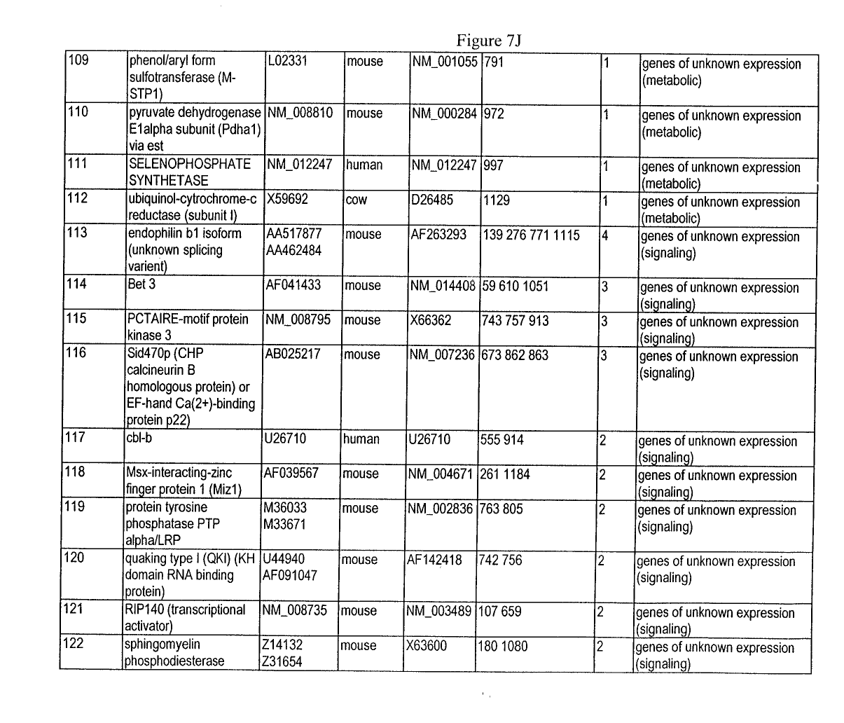

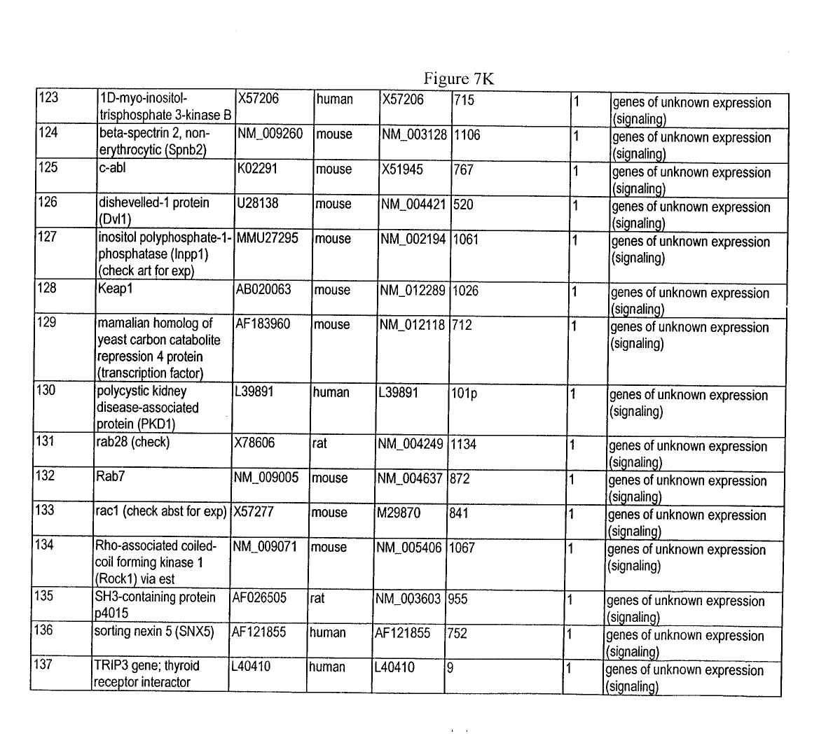

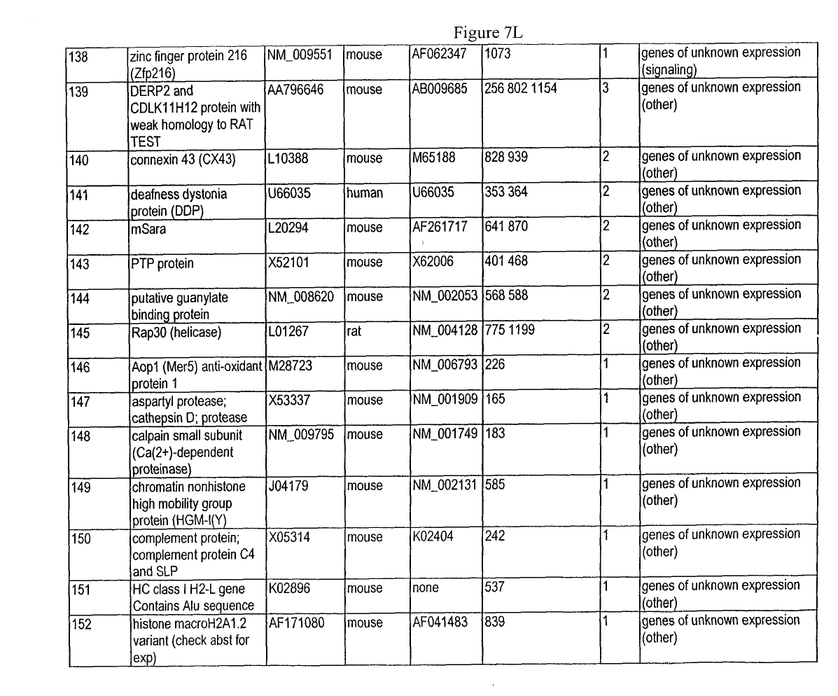

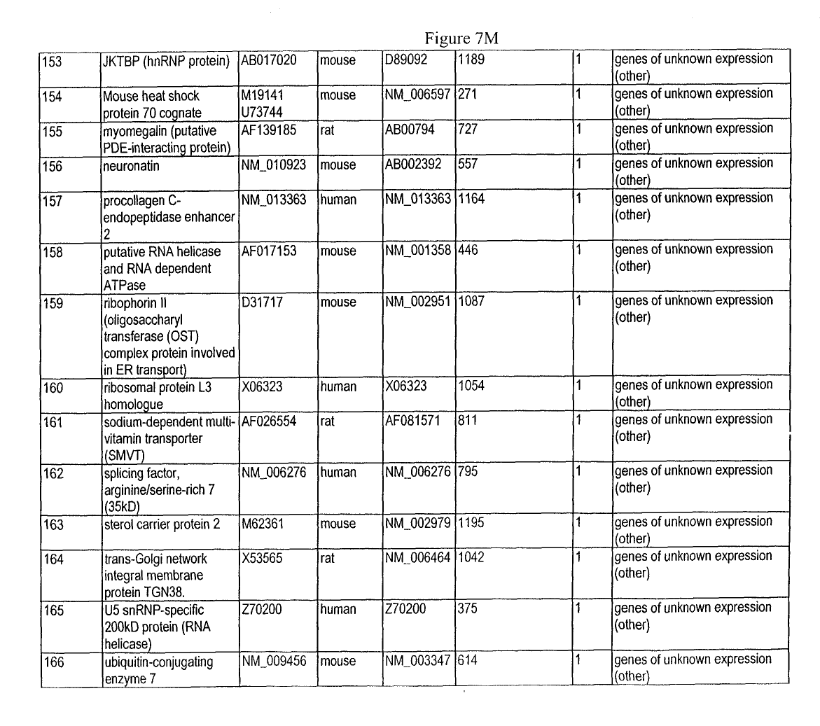

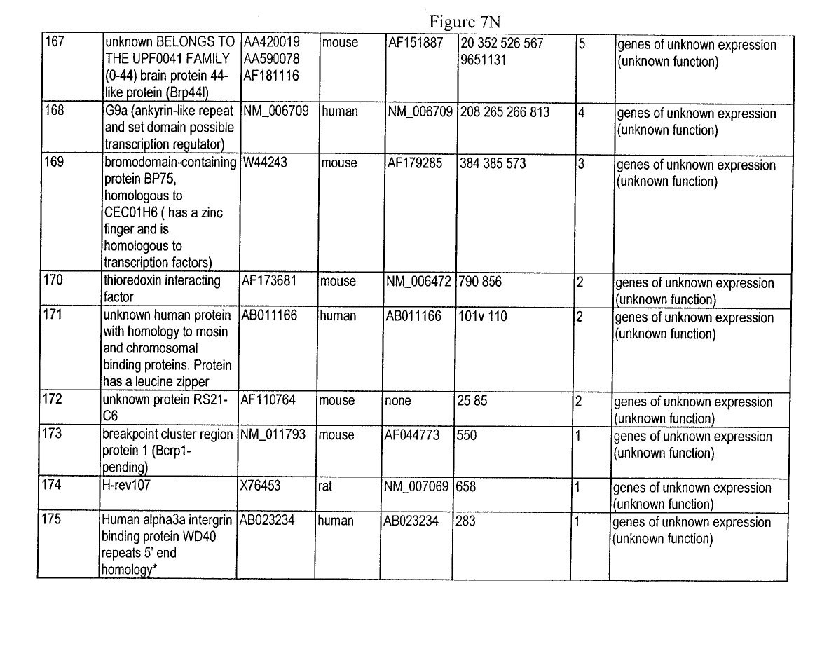

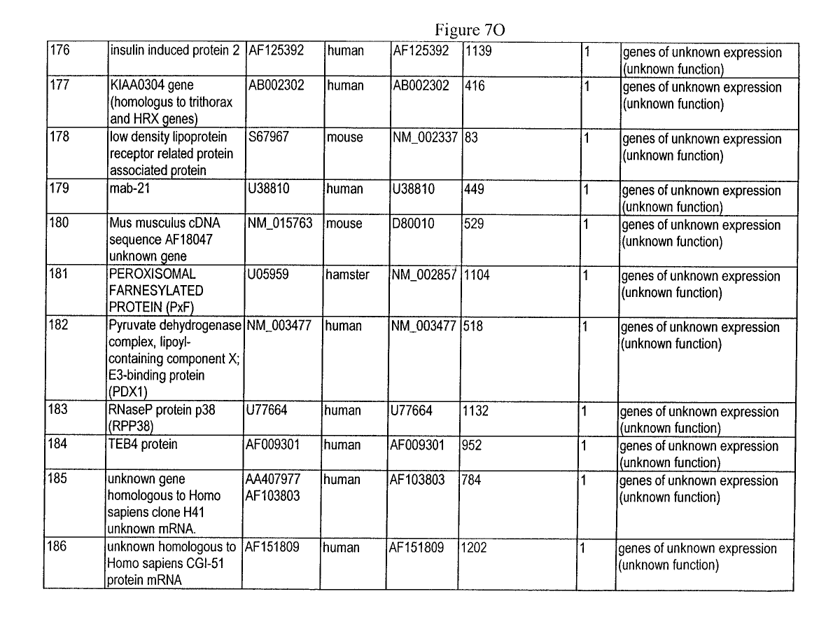

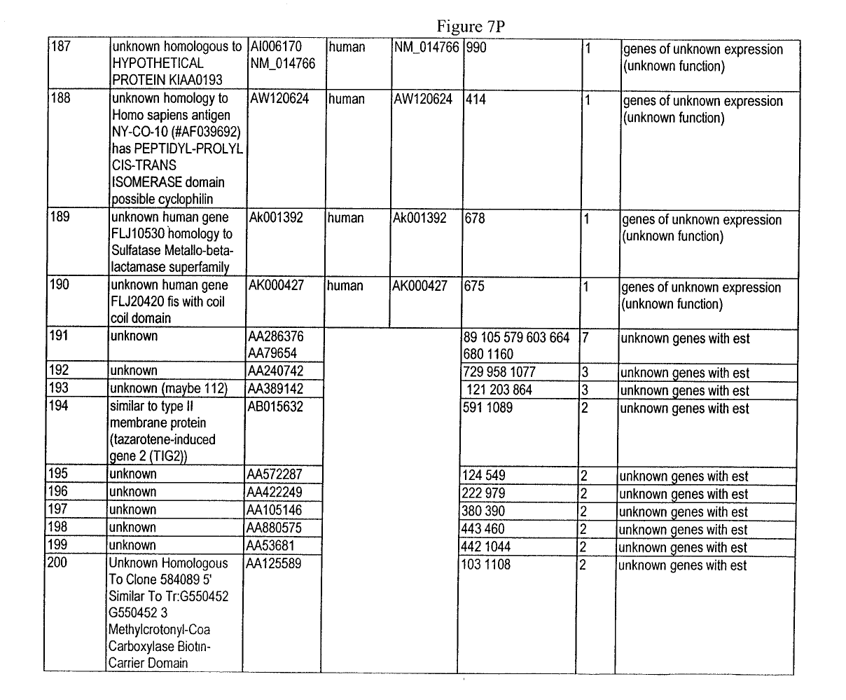

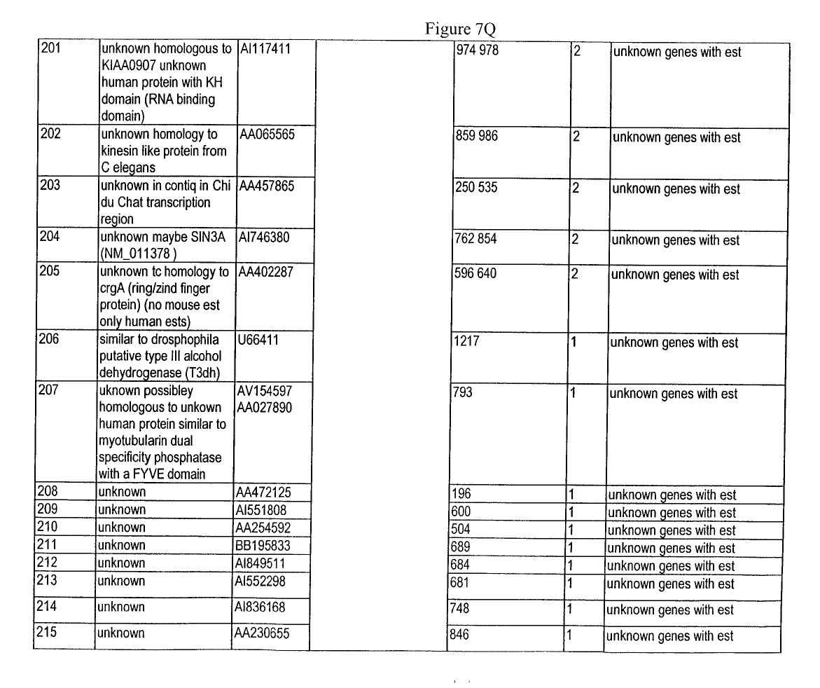

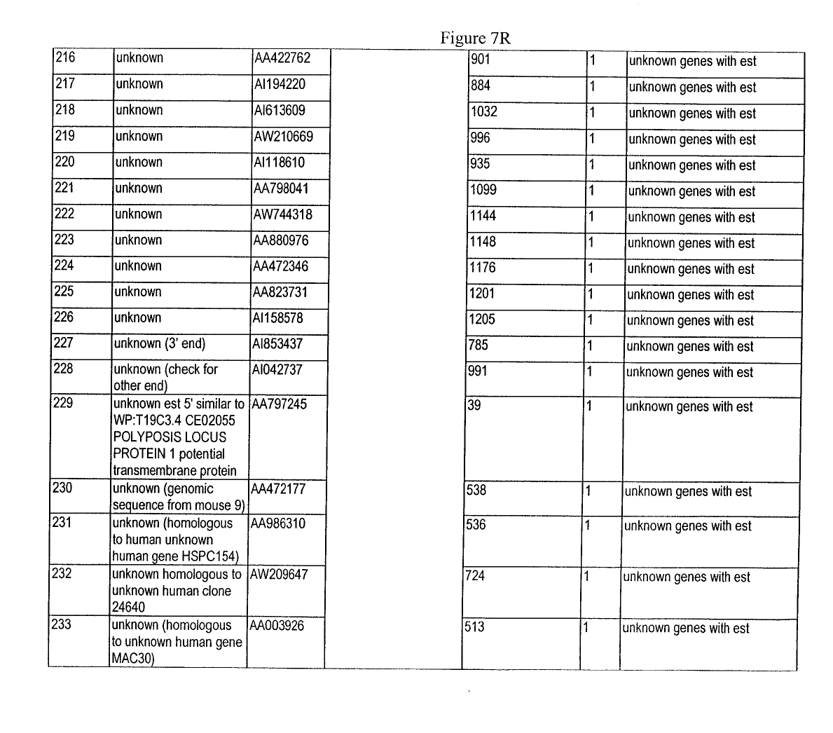

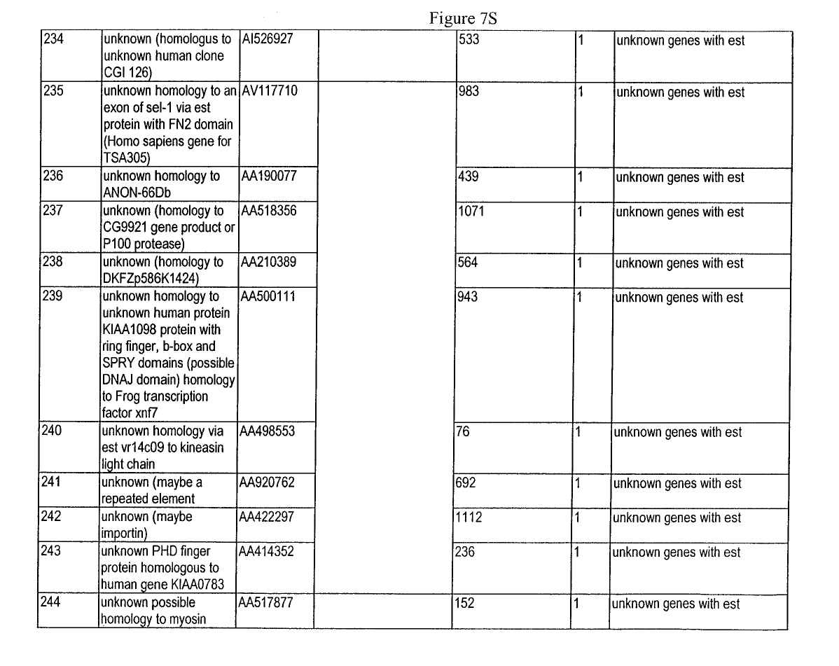

Sequences in the MAU library fall into three categories: (1) novel sequences (FIG. 1); (2) sequences from genes for which at least partial sequences were known, but for which no function was known or predicted (FIGS. 2A-2R and 3A-3E); and (3) sequences of genes with a known or predicted function (included in FIGS. 6A-6E and 7A-7U). The novel sequences are designated c0148 (SEQ ID NO: 1), c0827 (SEQ ID NO: 2), and c1083 (SEQ ID NO: 3), and they are set forth in FIG. 1.

Some of the library sequences are a novel combination of sequences based on partial sequencing of genes that were identified in the Adipocyte Subtractive library as differentially expressed in adipocyte and fibroblast cells combined with overlapping sequences that were obtained from databanks (GenBank and TIGR (The Institute for Genomic Research)). Additional library sequences are novel combinations of sequences based on partial sequencing of genes that are identified in the Muscle Adipocyte Union Library as differentially expressed in both adipocyte and muscle cells combined with overlapping sequences that were obtained from the databanks. Genes in these categories include b0117 (AAPT-like protein with CDP-alcohol phosphatidyltransferases signature sequence; SEQ ID NO: 81), b0175 (GS2 protein; SEQ ID NO: 87), c0139 (endophilin-like protein coil-coil plus SH3 domain; SEQ ID NO: 12), c0250 (SEQ ID NO: 17), c0352 (SEQ ID NO: 18), c0582 (Rab GTPase domain; SEQ ID NO: 33), c0591 (isoform of TIG2 protein; SEQ ID NO: 34), and c0840 (Clu-like protein; SEQ ID NO: 53). These sequences are depicted in FIGS. 2A-2R and 3A-3E, and are particularly useful in the methods of the invention.

Sequences that are differentially expressed in adipocytes, muscle cells, or both (as compared to expression in, e.g., fibroblasts) are useful, e.g., as genes or providing gene products that are targets for treatments for disorders involving glucose transport and for diagnosis of disorders involving aberrant glucose transport such as type II diabetes.

DNA Arrays

DNAs containing complete or partial sequences from the library of glucose transport-related sequences can be used to construct conventional DNA arrays (sometimes called DNA chips or gene chips). A DNA array according to the invention can contain tens, hundreds, or thousands of individual sequences immobilized (tethered) at discrete, predetermined locations (addresses or “spots”) on a solid, planar support, e.g., glass or nylon. Each spot may contain more than one DNA molecule, but each DNA molecule at a given address has an identical nucleotide sequence. The DNA array can be a macroarray or microarray, the difference being in the size of the DNA spots. Macroarrays contain spots of about 300 microns in diameter or larger and can be imaged using gel or blot scanners. Microarrays contain spots less than 300 microns, typically less than 200 microns, in diameter.

For analysis and comparison of glucose transport-related gene expression patterns, an array is constructed using sequences from at least four, e.g., at least 10, 20, 40, 60, 80 or 100 genes in the above-described library. A population of labeled cDNA representing total mRNA from a sample of a tissue of interest, e.g., muscle or adipose tissue, is contacted with the DNA array under suitable hybridization conditions. Hybridization of cDNAs with sequences in the array is detected, e.g., by fluorescence at particular addresses on the solid support. Thus, a pattern of fluorescence representing a gene expression pattern in the tissue of a particular individual or group of individuals is obtained. These patterns of glucose transport-related gene expression can be digitized and stored electronically for computerized analysis and comparison. For example, an array according to the invention can be used to compare glucose transport-related gene expression of type II diabetic individuals with each other, and with non-diabetic individuals. Such comparisons will reveal specific genes whose expression is increased or decreased in a given tissue type in individuals with type II diabetes or other glucose transport-related diseases or disorders. Such arrays can also be used to diagnose individuals having or at risk for a glucose transport-related disorder such as type II diabetes. For example, a nucleic acid sample (e.g., cDNA) from an individual suspected of having a glucose transport-related disorder is prepared and hybridized to the array. The pattern (including the level) of expression of sequences in the sample is compared to a reference pattern (e.g., representing the pattern of expression in unaffected individuals, and/or representing the pattern of expression in individuals known to have a particular glucose transport-related disorder). A pattern of expression in the sample that varies from that of the unaffected reference, and/or corresponds with the pattern of expression in a glucose transport disorder indicates that the individual has a glucose transport disorder.

In some embodiments of the invention, cDNAs are used to form the array. Suitable cDNAs can be obtained by conventional polymerase chain reaction (PCR) techniques. The length of the cDNAs can be from 20 to 2,000 nucleotides, e.g., from 100 to 1,000 nucleotides. Other methods known in the art for producing cDNAs can be used. For example, reverse transcription of a cloned sequence can be used (for example, as described in Sambrook et al., eds., Molecular Cloning: A Laboratory Manual. 2nd ed., Cold Spring Harbor Laboratory, Cold Spring Harbor Laboratory Press, Cold Spring Harbor, N.Y., 1989) The cDNAs are placed (“printed” or “spotted”) onto a suitable solid support (substrate), e.g., a coated glass microscope slide, at specific, predetermined locations (addresses) in a two-dimensional gild. A small volume, e.g., 5 nanoliters, of a concentrated DNA solution is used in each spot. Spotting can be carried out using a commercial microspotting device (sometimes called an arraying machine or gridding robot) according to the vendor's instructions. Commercial vendors of solid supports and equipment for producing DNA arrays include BioRobotics Ltd., Cambridge, UK; Corning Science Products Division, Acton, Mass.; GENPAK Inc., Stony Brook, N.Y.; SciMatrix, Inc., Durham, N.C.; and TeleChem International, Sunnyvale, Calif.

The cDNAs can be attached to the solid support by any suitable method. In general, the linkage is covalent. Suitable methods of covalently linking DNA molecules to the solid support include amino cross-linking and UV crosslinking. For guidance concerning construction of cDNA arrays according to the invention, see, e.g., DeRisi et al., 1996, Nature Genetics 14:457-460; Khan et al., 1999, Electrophoresis 20:223-229; Lockhart et al., 1996, Nature Biotechnol. 14:1675-1680.

In some embodiments of the invention, the immobilized DNAs in the array are synthetic oligonucleotides. Preformed oligonucleotides can be spotted to form a DNA array, using techniques described above with regard to cDNA. In general, however, the oligonucleotides are synthesized directly on the solid support. Methods for synthesizing oligonucleotide arrays are known in the art. See, e.g., Fodor et al., U.S. Pat. No. 5,744,305. The sequences of the oligonucleotides represent portions of the sequences in the library described above. For example, the lengths of oligonucleotides are 10 to 50 nucleotides, e.g., 15, 20, 25, 30, 35, 40, or 45 nucleotides.

In some embodiments of the invention, the human homologs of the identified sequences are used in the detection method. Examples of such human homologs are listed with their GenBank accession numbers in FIGS. 6A-6E, 7A-7U, and 8A-8I. In other embodiments, the sequence used for detection consists of highly conserved regions of the sequence, e.g., sequence that is highly conserved between homologous mouse and human sequence.

Sample Preparation and Analysis

In methods of the invention, the transcription level of a glucose transport-related gene is assumed to be reflected in the amount of its corresponding mRNA present in cells of assayed tissue or cell lines derived from specific tissues. In general, mRNA from the cells or tissue is copied into cDNA under conditions such that the relative amounts of cDNA produced representing specific genes reflect the relative amounts of the mRNA in the sample. Comparative hybridization methods involve comparing the amounts of various, specific mRNAs in two tissue samples, as indicated by the amounts of corresponding cDNAs hybridized to sequences from the glucose transport-related gene library.

The mRNA used to produce cDNA is generally isolated from other cellular contents and components. One useful approach for mRNA isolation is a two-step approach. In the first step, total RNA is isolated. The second step is based on hybridization of the poly(A) tails of mRNAs to oligo(dT) molecules bound to a solid support, e.g., a chromatographic column or magnetic beads. Total RNA isolation and mRNA isolation are known in the art and can be accomplished, for example, using commercial kits according to the vendor's instructions. Similarly, synthesis of cDNA from isolated mRNA is known in the art and can be accomplished using commercial kits according to the vendor's instructions. Fluorescent labeling of cDNA can be achieved by including a fluorescently labeled deoxynucleotide, e.g., Cy5-dUTP or Cy3-dUTP, in the cDNA synthesis reaction. For guidance concerning isolation of mRNA and synthesis of fluorescently labeled cDNA for analysis on a DNA array, see, e.g., Ross et al., 2000, Nature Genetics 24:227-235.

In the invention, conventional techniques for hybridization and washing of DNA arrays, detection of hybridization, and data analysis can be employed routinely without undue experimentation. Commercial vendors of hardware and software for scanning DNA arrays and analyzing data include Cartesian Technologies, Inc. (Irvine, Calif.); GSI Lumonics (Watertown, Mass.); Genetic Microsystems Inc. (Woburn, Mass.); and Scanalytics, Inc. (Fairfax, Va.).

Isolated Nucleic Acid Molecules

The invention provides certain novel, isolated nucleic acids that encode murine glucose transport-related polypeptides, or biologically active portions thereof (FIG. 1). In addition to forming part of the library, these nucleic acids can be used as hybridization probes to identify the full-length genes that they represent, and to isolate related nucleic acids, e.g., murine nucleic acids can be used to identify and clone human homologs. These nucleic acids also can be used to design PCR primers for PCR amplification of related nucleic acid molecules. The full-length genes identified and isolated using these novel sequences are predicted to function in insulin-responsive glucose transport systems in mammalian muscle cells and adipose cells.

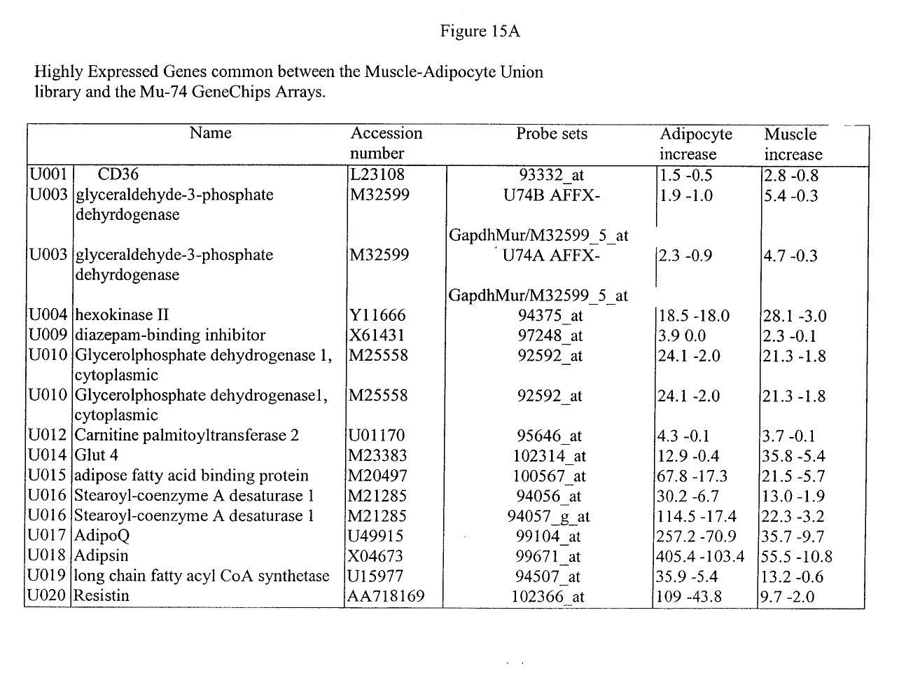

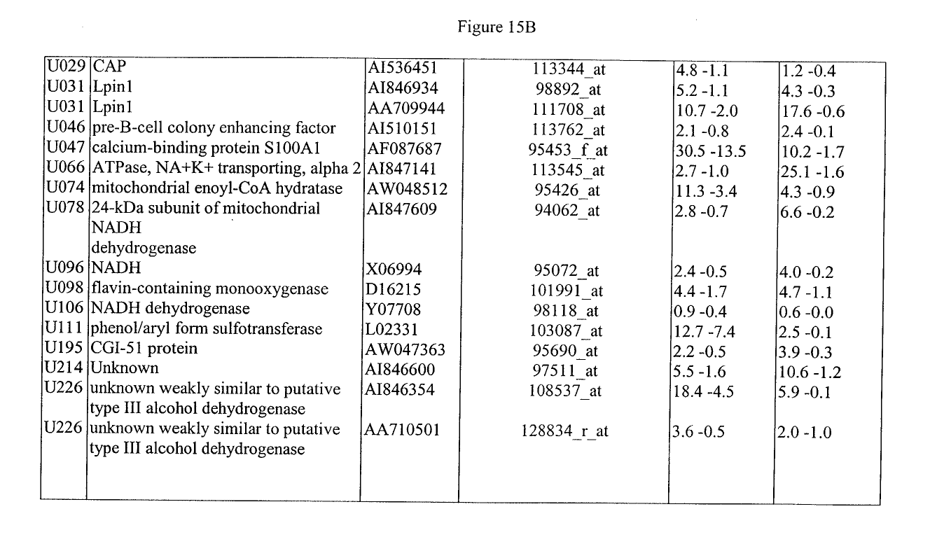

As used herein, “isolated DNA” means DNA that has been separated from DNA that flanks the DNA in the genome of the organism in which the DNA naturally occurs. The term therefore includes recombinant DNA incorporated into a vector, e.g., a cloning vector or an expression vector. The term also includes a molecule such as a cDNA, a genomic fragment, a fragment produced by PCR, or a restriction fragment. The term also includes a recombinant nucleotide sequence that is part of a hybrid gene construct, i.e., a construct encoding a fusion protein. The term excludes an isolated chromosome. Isolated nucleic acids of the invention (e.g., SEQ ID NOS: 1-93) can include modifications at the 3′ and/or 5′ end of the molecule including a metal, a modified nucleotide residue, or a nucleotide sequence that is not contiguous with the sequence of interest in nature. Such modifications can also be made to the sequences or fragments of sequences used in the invention (e.g., sequences derived from the genes listed in FIGS. 6-9 and 13-15).

A full length coding sequence that contains a novel nucleotide sequence of the invention, e.g., a nucleic acid molecule containing a sequence set forth in FIG. 1, or a complement thereof, can be isolated using conventional molecular biology techniques and the sequence information provided herein. For example the isolation can be accomplished without undue experimentation by applying techniques described in numerous treatises and reference manuals. For general guidance and specific protocols, see, e.g., Sambrook et al., eds., Molecular Cloning: A Laboratory Manual, 2nd ed., Cold Spring Harbor Laboratory, Cold Spring Harbor Laboratory Press, Cold Spring Harbor, N.Y., 1989; Ausubel et al. (eds.), 1994, Current Protocols in Molecular Biology, John Wiley & Sons, Inc.; Innes et al. (eds.), 1990, PCR Protocols, Academic Press.

A nucleic acid molecule of the invention can be amplified using cDNA, mRNA, or genomic DNA as a template and appropriate oligonucleotide primers according to standard PCR amplification techniques. Once isolated, the full-length nucleic acid can be cloned into an appropriate vector and characterized by conventional DNA sequence analysis, using standard techniques and equipment.

A nucleic acid fragment encoding a biologically active portion of a polypeptide encoded by a novel nucleic acid of the invention can be identified and prepared by isolating a portion of any of the sequences useful in the invention, expressing the encoded portion of the polypeptide protein (e.g., by recombinant expression in vitro) and assessing the activity of the encoded portion of the polypeptide.

The invention further encompasses nucleic acid molecules that differ from the nucleotide sequence set forth in FIG. 1, due to degeneracy of the genetic code and thus encode the same amino acid sequence as that encoded by the nucleotide sequence set forth in FIG. 1. The invention further encompasses isolated nucleic acid molecules that hybridize with the sequences set forth in FIG. 1 under high stringency conditions. As used herein, “high stringency” means the following: hybridization at 42° C. in the presence of 50% formamide; a first wash at 65° C. with 2×SSC containing 1% SDS; followed by a second wash at 65° C. with 0.1×SSC.

In addition to the nucleotide sequences set forth in FIG. 1, it will be appreciated by those skilled in the art that DNA sequence polymorphisms that lead to changes in the amino acid sequence may exist within a population (e.g., the human population). Such genetic polymorphisms may exist among individuals within a population due to natural allelic variation. An allele is one of a group of genes that occur alternatively at a given genetic locus. As used herein, “allelic variation” means variation in a nucleotide sequence that occurs at a given locus, or variation in an amino acid sequence of a polypeptide encoded by the nucleotide sequence at a given locus. Alternative alleles can be identified by sequencing the gene of interest in a number of different individuals. This can be accomplished by using hybridization probes to identify nucleic acids corresponding to the same genetic locus in a variety of individuals. The nucleic acid is then sequenced (e.g., amplified using PCR and the PCR products are sequenced) to identify variations. Isolated nucleic acids containing the nucleotide sequences of FIG. 1 that display allelic variations while retaining functional activity are within the scope of the invention.

In some embodiments of the invention, changes are introduced into the sequences of FIG. 1 by mutation thereby leading to changes in the amino acid sequence of the encoded protein, without altering the biological activity of the protein. For example, one can make nucleotide substitutions leading to amino acid substitutions at non-essential amino acid residues. A non-essential amino acid residue is a residue that can be altered from the wild-type sequence without altering the biological activity of the gene product (e.g., a protein). For example, amino acid residues that are not conserved or only semi-conserved among homologs of various species may be non-essential for activity and thus would be likely targets for alteration. In contrast, amino acid residues that are conserved among the homologs of various species (e.g., murine and human) may be necessary for activity and thus would not be likely targets for alteration.

An isolated nucleic acid molecule encoding a variant protein can be created by introducing one or more nucleotide substitutions, additions, or deletions into the nucleotide sequence of c0148 (SEQ ID NO: 1), c0827 (SEQ ID NO: 2), and c1083 (SEQ ID NO: 3) such that one or more amino acid substitutions, additions, or deletions are introduced into the encoded protein. Mutations can be introduced by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed recombinantly and the activity of the protein can be determined.

Isolating Homologous Sequences from Other Species

The human homologs of glucose-transport related genes and their products are useful for various embodiments of the present invention including diagnosis of glucose transport-related disorders such as type II diabetes. Homologs have already been identified for certain genes and GenBank Accession numbers are supplied for these. In those cases where a human homolog is not identified, several approaches can be used to identify such genes. These methods include low stringency hybridization screens of human libraries with a mouse glucose transport-related nucleic acid sequence, polymerase chain reactions (PCR) of human DNA sequence primed with degenerate oligonucleotides derived from a mouse glucose transport-related gene, two-hybrid screens, and database screens for homologous sequences.

Antisense Nucleic Acids

The invention includes antisense nucleic acid molecules, i.e., nucleic acid molecules whose nucleotide sequence is complementary to all or part of an mRNA based on the sequences c0148, c0827, and c1083 (FIG. 1). An antisense nucleic acid molecule can be antisense to all or part of a non-coding region of the coding strand of a nucleotide sequence encoding a polypeptide of the invention. The non-coding regions (“5′ and 3′ untranslated regions”) are the 5′ and 3′ sequences that flank the coding region and are not translated into amino acids.

An antisense oligonucleotide can be, for example, about 5, 10, 15, 20, 25, 30, 35, 40, 45, or 50 nucleotides or more in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention can be administered to a mammal, e.g., a human patient. Alternatively, they can be generated in sittu such that they hybridize with or bind to cellular mRNA and/or genomic DNA encoding a selected polypeptide of the invention to thereby inhibit expression, e.g., by inhibiting transcription and/or translation. The hybridization can be by conventional nucleotide complementarities to form a stable duplex, or, for example, in the case of an antisense nucleic acid molecule which binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies that bind to cell surface receptors or antigens. The antisense nucleic acid molecules can also be delivered to cells using the vectors described herein. For example, to achieve sufficient intracellular concentrations of the antisense molecules, vector constructs can be used in which the antisense nucleic acid molecule is placed under the control of a strong pol II or pol III promoter.

An antisense nucleic acid molecule of the invention can be an α-anomeric nucleic acid molecule. An α-anomeric nucleic acid molecule forms specific double-stranded hybrids with complementary RNA in which, contrary to the usual, β-units, the strands run parallel to each other (Gaultier et al., 1987, Nucleic Acids Res. 15:6625-6641). The antisense nucleic acid molecule can also comprise a 2′-o-methylribonucleotide (Inoue et al., 1987, Nucleic Acids Res. 15:6131-6148) or a chimeric RNA-DNA analog (Inoue et al., 1987, FEBS Lett. 215:327-330).

Antisense molecules that are complementary to all or part of a glucose transport-related gene are also useful for assaying expression of such genes using hybridization methods known in the art. For example, the antisense molecule is labeled (e.g., with a radioactive molecule) and an excess amount of the labeled antisense molecule is hybridized to an RNA sample. Unhybridized labeled antisense molecule is removed (e.g., by washing) and the amount of hybridized antisense molecule measured. The amount of hybridized molecule is measured and used to calculate the amount of expression of the glucose transport-related gene. In general, antisense molecules used for this purpose can hybridize to a sequence from a glucose transport-related gene under high stringency conditions such as those described herein. When the RNA sample is first used to synthesize cDNA, a sense molecule can be used. It is also possible to use a double-stranded molecule in such assays as long as the double-stranded molecule is adequately denatured prior to hybridization.

Ribozymes

The invention also encompasses ribozymes that have specificity for the sequences c0148, c0827, and c1083. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes (described in Haselhoff and Gerlach, 1988, Nature 334:585-591)) can be used to catalytically cleave mRNA transcripts to thereby inhibit translation of the protein encoded by the mRNA. A ribozyme having specificity for a nucleic acid molecule of the invention can be designed based upon the nucleotide sequence of a cDNA disclosed herein. For example, a derivative of a Tetrahymena L-19 IVS RNA can be constructed in which the nucleotide sequence of the active site is complementary to the nucleotide sequence to be cleaved in a glucose transport-related mRNA (Cech et al. U.S. Pat. No. 4,987,071; and Cech et al., U.S. Pat. No. 5,116,742). Alternatively, an mRNA encoding a polypeptide of the invention can be used to select a catalytic RNA having a specific ribonuclease activity from a pool of RNA molecules. See, e.g., Bartel and Szostak, 1993, Science 261:1411-1418.

The invention also encompasses nucleic acid molecules that form triple helical structures. For example, expression of a polypeptide of the invention can be inhibited by targeting nucleotide sequences complementary to the regulatory region of the gene encoding the polypeptide (e.g., the promoter and/or enhancer) to form triple helical structures that prevent transcription of the gene in target cells. See generally Helene, 1991, Anticancer Drug Des. 6(6):569-84; Helene, 1992, Ann. N.Y. Acad. Sci. 660:27-36; and Maher, 1992, Bioassays 14(12):807-15.

In various embodiments, the nucleic acid molecules of the invention can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (see Hyrup et al., 1996, Bioorganic & Medicinal Chemistry 4(1): 5-23). Peptide nucleic acids (PNAs) are nucleic acid mimics, e.g., DNA mimics, in which the deoxyribose phosphate backbone is replaced by a pseudopeptide backbone and only the four natural nucleobases are retained. The neutral backbone of PNAs allows for specific hybridization to DNA and RNA under conditions of low ionic strength. The synthesis of PNA oligomers can be performed using standard solid phase peptide synthesis protocols, e.g., as described in Hyrup et al., 1996, supra; Perry-O'Keefe et al., 1996, Proc. Natl. Acad. Sci. USA 93: 14670-675.

PNAs can be used in therapeutic and diagnostic applications. For example, PNAs can be used as antisense or antigene agents for sequence-specific modulation of gene expression by, e.g., inducing transcription or translation arrest or inhibiting replication. PNAs can also be used, e.g., in the analysis of single base pair mutations in a gene by, e.g., PNA directed PCR clamping; as artificial restriction enzymes when used in combination with other enzymes, e.g., S1 nucleases (Hyrup, 1996, supra; or as probes or primers for DNA sequence and hybridization (Hyrup, 1996, supra; Perry-O'Keefe et al., 1996, Proc. Natl. Acad. Sci. USA 93: 14670-675).

PNAs can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras can be generated which may combine the advantageous properties of PNA and DNA. Such chimeras allow DNA recognition enzymes, e.g., RNAse H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup, 1996, supra). The synthesis of PNA-DNA chimeras can be performed as described in Hyrup, 1996, supra, and Finn et al., 1996, Nitcleic Acids Res. 24:3357-63. For example, a DNA chain can be synthesized on a solid support using standard phosphoramidite coupling chemistry and modified nucleoside analogs. Compounds such as 5′-(4-methoxytrityl)amino-5′-deoxy-thymidine phosphoramidite can be used as a link between the PNA and the 5′ end of DNA (Mag et al., 1989, Nucleic Acids Res. 17:5973-88). PNA monomers are then coupled in a stepwise manner to produce a chimeric molecule with a 5′ PNA segment and a 3′ DNA segment (Finn et al., 1996, Nucleic Acids Res. 24:3357-63). Alternatively, chimeric molecules can be synthesized with a 5′ DNA segment and a 3′ PNA segment (Peterser et al., 1975, Bioorganic Med. Chem. Lett. 5:1119-11124).

In some embodiments, the oligonucleotide includes other appended groups such as peptides (e.g., for targeting host cell receptors in vivo), or agents facilitating transport across the cell membrane (see, e.g., Letsinger et al., 1989, Proc. Natl. Acad. Sci. USA 86:6553 -6556; Lemaitre et al., 1987, Proc. Natl. Acad. Sci. USA 84:648-652; PCT Publication No. WO 88/09810) or the blood-brain barrier (see, e.g., PCT Publication No. WO 89/10134). In addition, oligonucleotides can be modified with hybridization-triggered cleavage agents (see, e.g., Krol et al., 1988, Bio/Techniques 6:958-976) or intercalating agents (see, e.g., Zon, 1988, Pharm. Res. 5:539-549). To this end, the oligonucleotide may be conjugated to another molecule, e.g., a peptide, hybridization triggered cross-linking agent, transport agent, hybridization-triggered cleavage agent, etc.

Isolated Proteins

The invention provides isolated polypeptides encoded by glucose transport-related nucleic acids depicted in FIGS. 1, 2A-2R, and 3A-3E. These polypeptides can be used, e.g., as immunogens to raise antibodies. Methods are well known in the art for predicting the translation products of the nucleic acids (i.e, using computer programs that provide the predicted polypeptide sequences and direction as to which of the three reading frames is the open reading frame of the sequence. These polypeptide sequences can then be produced either biologically (e.g., by positioning the nucleic acid sequence that encodes them in-frame in an expression vector transfected into a compatible expression system) or chemically using methods known in the art. The polypeptides encoded by the genes listed in FIGS. 6-9 and 13-15 are also useful in the invention. For example, the entire polypeptide or a fragment thereof can be used to produce an antibody that is useful in a screening assay. FIGS. 6-9 and 13-15, provide the GenBank accession numbers of the sequences, when available. These listings provide both nucleotide and polypeptide sequences that are useful in the invention.

An “isolated” or “purified” protein or biologically active portion thereof is substantially free of cellular material or other contaminating proteins from the cell or tissue source from which the protein is derived, or substantially free of chemical precursors or other chemicals when chemically synthesized. The language “substantially free of cellular material” includes preparations of protein in which the protein is separated from cellular components of the cells from which it is isolated or recombinantly produced. Thus, protein that is substantially free of cellular material includes preparations of protein having less than about 30%, 20%, 10%, or 5% (by dry weight) of heterologous protein (also referred to herein as “contaminating protein”). In general, when the protein or biologically active portion thereof is recombinantly produced, it is also substantially free of culture medium, i.e., culture medium represents less than about 20%, 10%, or 5% of the volume of the protein preparation. In general, when the protein is produced by chemical synthesis, it is substantially free of chemical precursors or other chemicals, i.e., it is separated from chemical precursors or other chemicals that are involved in the synthesis of the protein. Accordingly such preparations of the protein have less than about 30%, 20%, 10%, 5% (by dry weight) of chemical precursors or compounds other than the polypeptide of interest.

Expression of proteins and polypeptides can be assayed to determine the amount of expression. Methods for assaying protein expression are known in the art and include Western blot, immunoprecipitation, and radioimmunoassay.

Biologically active portions of a polypeptide of the invention include polypeptides comprising amino acid sequences sufficiently identical to or derived from the amino acid sequence of the protein, which include fewer amino acids than the full length protein, and exhibit at least one activity of the corresponding full-length protein. Typically, biologically active portions comprise a domain or motif with at least one activity of the corresponding protein. A biologically active portion of a protein of the invention can be a polypeptide which is, for example, 10, 25, 50, 100, or more amino acids in length. Moreover, other biologically active portions, in which other regions of the protein are deleted, can be prepared by recombinant techniques and evaluated for one or more of the functional activities of the native form of a polypeptide of the invention.

Polypeptides of the invention have the predicted amino acid sequence of an open reading frame of c0148 (SEQ ID NO: 1), c0827 (SEQ ID NO: 2), and c1083 (SEQ ID NO: 3). In some embodiments, polypeptides of the invention have the predicted amino acid sequence selected from SEQ ID NOS: 4-93. Other useful proteins are substantially identical (e.g., at least about 45%, preferably 55%, 65%, 75%, 85%, 95%, or 99%) to the predicted amino acid sequence of a polypeptide encoded by a polynucleotide comprising the polynucleotide sequence of c0148 (SEQ ID NO: 1), c0827 (SEQ ID NO: 2), and c1083 (SEQ ID NO: 3) or substantially identical (e.g., at least about 93%, preferably 94%, 95%, 96%, or 99%) to the predicted amino acid sequence of a polypeptide encoded by a polynucleotide comprising the polynucleotide sequence of c0148 (SEQ ID NO: 1), c0827 (SEQ ID NO: 2), and c1083 (SEQ ID NO: 3), and retain the functional activity of the protein of the corresponding naturally-occurring protein yet differ in amino acid sequence due to natural allelic variation or mutagenesis.

The comparison of sequences and determination of percent identity between two sequences can be accomplished using a mathematical algorithm. In an embodiment of the invention, the percent identity between two amino acid sequences is determined using the Needleman and Wunsch ((1970) J. Mol. Biol. 48:444-453) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In another embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5,-or 6. In general, percent identity between amino acid sequences referred to herein is determined using the BLAST 2.0 program, which is available to the public at http://www.ncbi.nlm.nih.gov/BLAST. Sequence comparison is performed using an ungapped alignment and using the default parameters (Blossum 62 matrix, gap existence cost of 11, per residue gap cost of 1, and a lambda ratio of 0.85). The mathematical algorithm used in BLAST programs is described in Altschul et al., 1997, Nucleic Acids Research 25:3389-3402.

The invention also provides chimeric or fusion proteins. As used herein, a “chimeric protein” or “fusion protein” comprises all or part (e.g., a biologically active portion) of a polypeptide of the invention operably linked to a heterologous polypeptide (i.e., a polypeptide other than the same polypeptide of the invention). Within the fusion protein, the term “operably linked” is intended to indicate that the polypeptide of the invention and the heterologous polypeptide are fused in-frame to each other. The heterologous polypeptide can be fused to the N-terminus or C-terminus of the polypeptide of the invention.

One useful fusion protein is a GST fusion protein in which the polypeptide of the invention is fused to the C-terminus of GST sequences. Such fusion proteins can facilitate the purification of a recombinant polypeptide of the invention.

In another embodiment, the fusion protein contains a heterologous signal sequence at its N-terminus. For example, the native signal sequence of a polypeptide of the invention can be removed and replaced with a signal sequence from another protein. For example, the gp67 secretory sequence of the baculovirus envelope protein can be used as a heterologous signal sequence (Current Protocols in Molecular Biology, Ausubel et al., eds., John Wiley & Sons, 1992). Other examples of eukaryotic heterologous signal sequences include the secretory sequences of melittin and human placental alkaline phosphatase (Stratagene; La Jolla, Calif.). In yet another example, useful prokaryotic heterologous signal sequences include the phoA secretory signal (Sambrook et al., stipra) and the protein A secretory signal (Pharmacia Biotech; Piscataway, N.J.).

In yet another embodiment, the fusion protein is an immunoglobulin fusion protein in which all or part of a polypeptide of the invention is fused to sequences derived from a member of the immunoglobulin protein family. The immunoglobulin fusion proteins of the invention can be incorporated into pharmaceutical compositions and administered to a subject to inhibit an interaction between a ligand (soluble or membrane-bound) and a protein on the surface of a cell (receptor), to thereby suppress signal transduction in vivo. The immunoglobulin fusion protein can be used to affect the bioavailability of a cognate ligand of a polypeptide of the invention. Inhibition of ligand/receptor interaction may be useful therapeutically, both for treating proliferative and differentiative disorders and for modulating (e.g., promoting or inhibiting) cell survival. Moreover, the immunoglobulin fusion proteins of the invention can be used as immunogens to produce antibodies directed against a polypeptide of the invention in a subject, to purify ligands and in screening assays to identify molecules which inhibit the interaction of receptors with ligands.

Chimeric and fusion proteins of the invention can be produced by standard recombinant DNA techniques. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, e.g., Ausubel et al., supra). Moreover, many expression vectors are commercially available that already encode a fusion moiety (e.g., a GST polypeptide). A nucleic acid encoding a polypeptide of the invention can be cloned into such an expression vector such that the fusion moiety is linked in-frame to the polypeptide of the invention.

A signal sequence of a polypeptide of the invention can be used to facilitate secretion and isolation of the secreted protein or other proteins of interest. Signal sequences are typically characterized by a core of hydrophobic amino acids which are generally cleaved from the mature protein during secretion in one or more cleavage events. Such signal peptides contain processing sites that allow cleavage of the signal sequence from the mature proteins as they pass through the secretory pathway. Thus, the invention pertains to the described polypeptides having a signal sequence, as well as to the signal sequence itself and to the polypeptide in the absence of the signal sequence (i.e., the cleavage products). In one embodiment, a nucleic acid sequence encoding a signal sequence of the invention can be operably linked in an expression vector to a protein of interest, such as a protein which is ordinarily not secreted or is otherwise difficult to isolate. The signal sequence directs secretion of the protein, such as from a eukaryotic host into which the expression vector is transformed, and the signal sequence is subsequently or concurrently cleaved. The protein can then be readily purified from the extracellular medium by methods known in the art. Alternatively, the signal sequence can be linked to the protein of interest using a sequence which facilitates purification, such as with a GST domain.

The present invention also pertains to variants of the polypeptides of the invention. Such variants have an altered amino acid sequence which can function as either agonists (mimetics) or as antagonists. Variants can be generated by mutagenesis, e.g., discrete point mutation or truncation. An agonist can retain substantially the same, or a subset, of the biological activities of the naturally occurring form of the protein. An antagonist of a protein can inhibit one or more of the activities of the naturally occurring form of the protein by. for example, competitively binding to a downstream or upstream member of a cellular signaling cascade which includes the protein of interest. Thus, specific biological effects can be elicited by treatment with a variant of limited function. Treatment of a subject with a variant having a subset of the biological activities of the naturally occurring form of the protein can have fewer side effects in a subject relative to treatment with the naturally occurring form of the protein.

Antibodies

An isolated polypeptide of the invention, or a fragment thereof, can be used as an immunogen to generate antibodies using standard techniques for polyclonal and monoclonal antibody preparation. The full-length polypeptide or protein can be used or, alternatively. the invention provides antigenic peptide fragments for use as immunogens. The antigenic peptide of a protein of the invention comprises at least 8 (e.g., 10, 15, 20, or 30) amino acid residues of the amino acid sequence of a sequence of the invention, e.g., c0148, c0827, and c1083, and encompasses an epitope of the protein such that an antibody raised against the peptide forms a specific immune complex with the protein. Sequences also useful in the invention include polypeptides encoded by the sequences in FIGS. 1, 2A-2R, and 3A-3E or polypeptides encoded by sequences comprising a sequence listed in FIGS. 1, 2A-2R, and 3A-3R. Polypeptides encoded by the known genes identified herein as glucose transport-related genes are also useful in the invention.

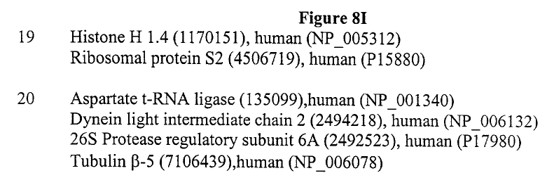

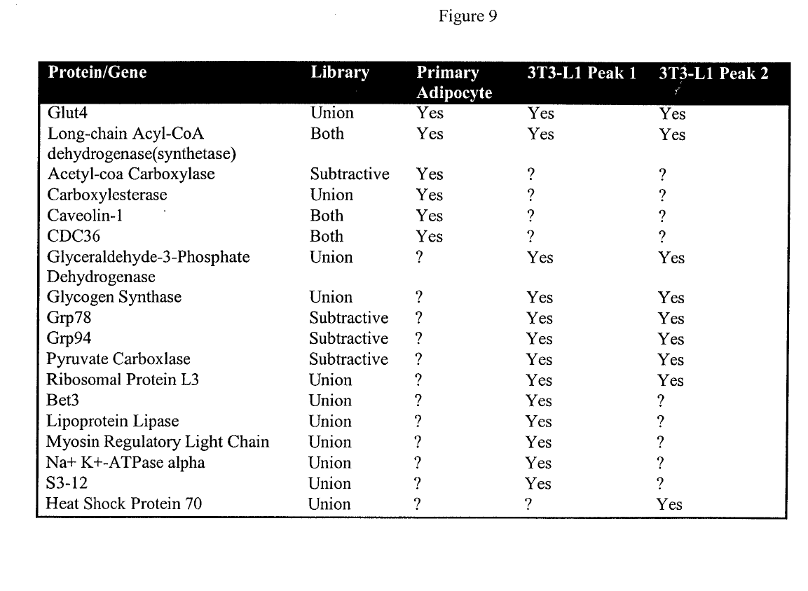

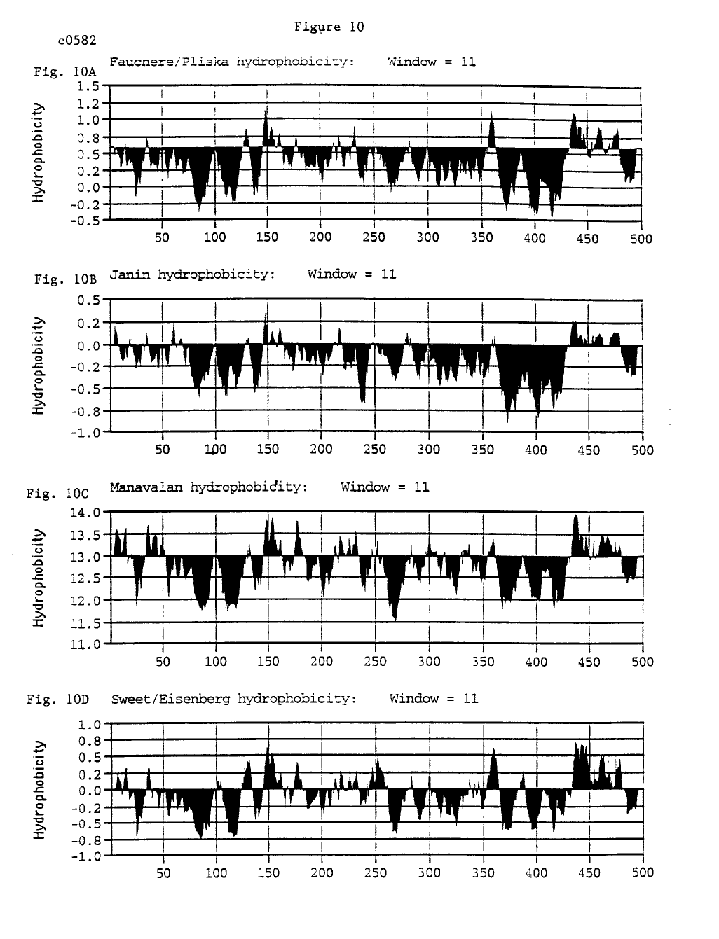

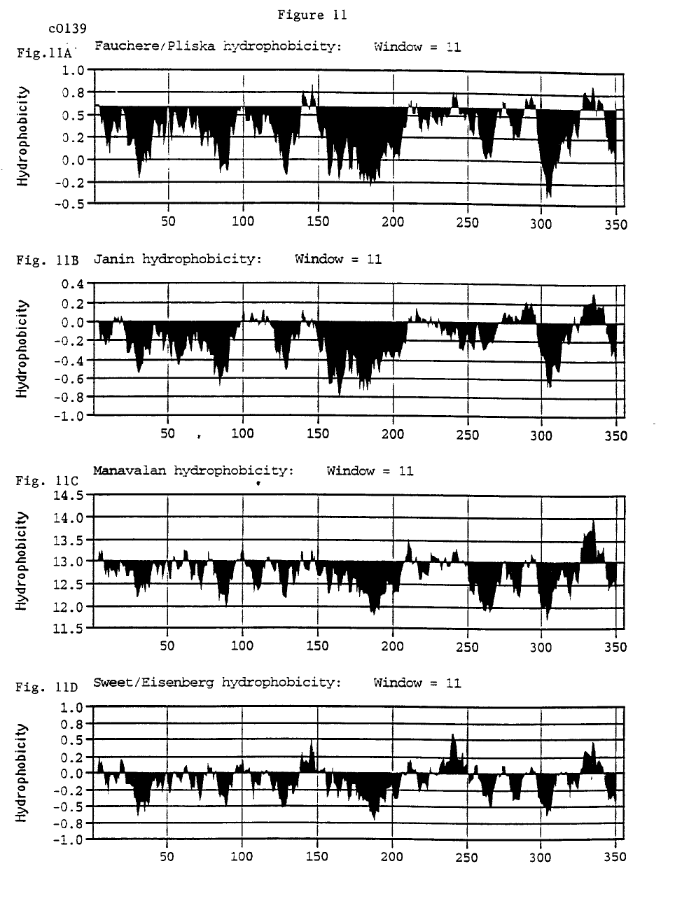

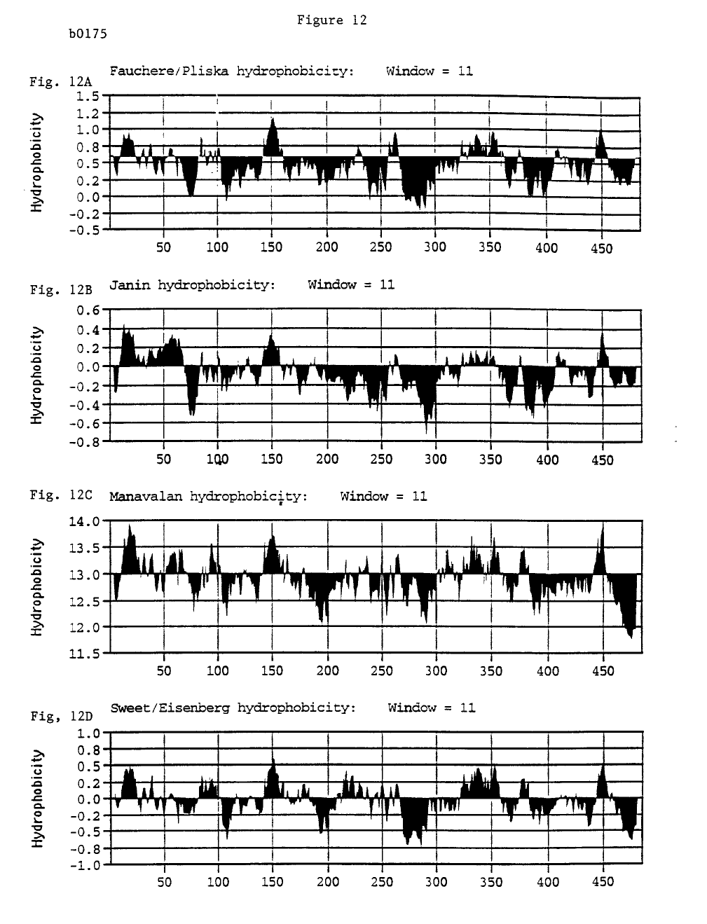

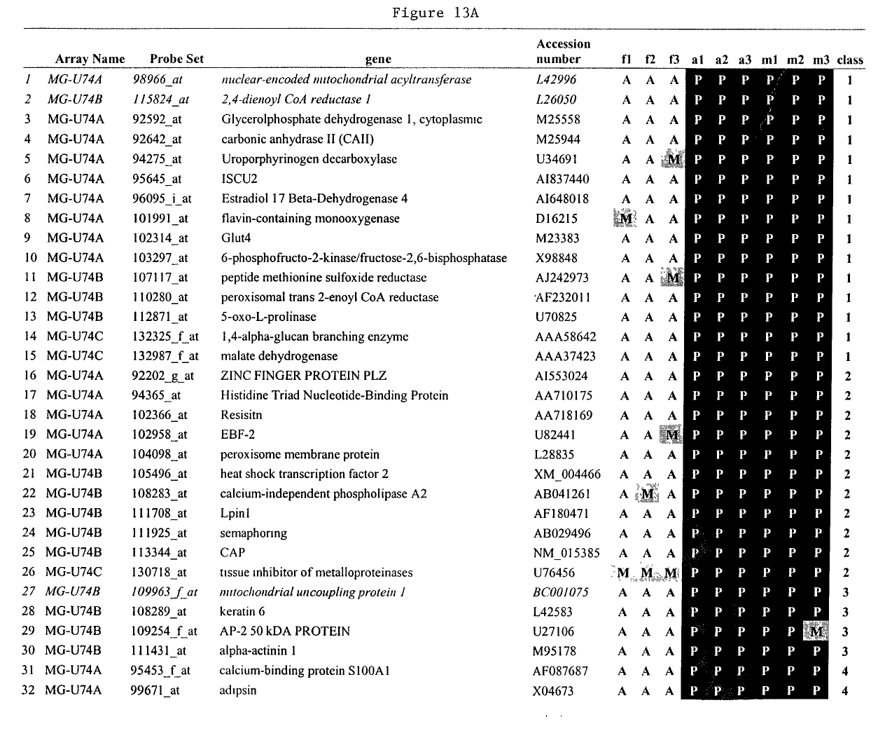

Epitopes can be encompassed by the antigenic peptide are regions that are located on the surface of the protein, e.g., hydrophilic regions. Hydrophilic regions of selected sequences are indicated in hydrophobicity plots (FIGS. 10A-10D, 11A-11D, and 12A-12D). These plots or similar analyses can be used to identify hydrophilic regions in polypeptides useful in the invention.

An immunogen typically is used to prepare antibodies by immunizing a suitable subject, (e.g., rabbit, goat, mouse or other mammal). An appropriate immunogenic preparation can contain, for example, a recombinantly expressed or a chemically synthesized polypeptide. The preparation can further include an adjuvant, such as Freund's complete or incomplete adjuvant, or similar immunostimulatory agent.

Polyclonal antibodies can be prepared as described above by immunizing a suitable subject with a polypeptide of the invention as an immunogen. The antibody titer in the immunized subject can be monitored over time by standard techniques, such as with an enzyme linked immunosorbent assay (ELISA) using immobilized polypeptide. If desired, the antibody molecules can be isolated from the mammal (e.g., from the blood) and further purified by well-known techniques, such as protein A chromatography to obtain the IgG fraction. At an appropriate time after immunization, e.g., when the specific antibody titers are highest, antibody-producing cells can be obtained from the subject and used to prepare monoclonal antibodies by standard techniques, such as the hybridoma technique originally described by Kohler and Milstein, 1975, Nature 256:495-497, the human B cell hybridoma technique (Kozbor et al., 1983, Immunol. Today 4:72), the EBV-hybridoma technique (Cole et al., 1985, Monoclonal Antibodies and Cancer Therapy, Alan R. Liss, Inc., pp. 77-96) or trioma techniques. The technology for producing hybridomas is well known (see generally Current Protocols in Immunology, 1994, Coligan et al. (eds.) John Wiley & Sons, Inc., New York, N.Y.). Hybridoma cells producing a monoclonal antibody of the invention are detected by screening the hybridoma culture supernatants for antibodies that bind the polypeptide of interest, e.g., using a standard ELISA assay.

Alternative to preparing monoclonal antibody-secreting hybridomas, a monoclonal antibody directed against a polypeptide of the invention can be identified and isolated by screening a recombinant combinatorial immunoglobulin library (e.g., an antibody phage display library) with the polypeptide of interest. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., 1991, Bio/Technology 9:1370-1372; Hay et al., 1992, Hum. Antibod. Hybridomas 3:81-85; Huse et al., 1989, Science 246:1275-1281; Griffiths et al., 1993, EMBO J. 12:725-734.

Additionally, recombinant antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al., 1988, Science 240:1041-1043; Liu et al., 1987, Proc. Natl. Acad. Sci. USA 84:3439-3443; Liu et al., 1987, J. Immunol. 139:3521-3526; Sun et al., 1987, Proc. Natl. Acad. Sci. USA 84:214-218; Nishimura et al., 1987, Canc. Res. 47:999-1005; Wood et al., 1985, Nature 314:446-449; and Shaw et al., 1988, J. Natl. Cancer Inst. 80:1553-1559); Morrison, 1985, Science 229:1202 -1207; Oi et al., 1986, Bio/Techniques 4:214; U.S. Pat. 5,225,539; Jones et al., 1986) Nature 321:552-525; Verhoeyan et al., 1988, Science 239:1534; and Beidler et al., 1988, J. Immunol. 141:4053-4060.

Completely human antibodies are particularly desirable for therapeutic treatment of human patients. Such antibodies can be produced using transgenic mice which are incapable of expressing endogenous immunoglobulin heavy and light chains genes, but which can express human heavy and light chain genes. The transgenic mice are immunized in the normal fashion with a selected antigen, e.g., all or a portion of a polypeptide of the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., U.S. Pat. Nos. 5,625,126; 5,633,425; 5,569,825; 5,661,016; and 5,545,806. In addition, companies such as Abgenix, Inc. (Freemont, Calif.), can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a murine antibody, is used to guide the selection of a completely human antibody recognizing the same epitope. (Jespers et al., 1994, Biotechnology 12:899-903).

An antibody directed against a polypeptide of the invention (e.g., monoclonal antibody) can be used to isolate the polypeptide by standard techniques, such as affinity chromatography or immunoprecipitation. Moreover, such an antibody can be used to detect the protein (e.g., in a cellular lysate or cell supernatant) in order to evaluate the abundance and pattern of expression of the polypeptide. The antibodies can also be used diagnostically to monitor protein levels in tissue as part of a clinical testing procedure, e.g., for example, determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Screening Assays

The invention provides a method for identifying modulators, i.e., candidate agents or reagents, of expression or activity of a glucose transport-related nucleic acid or polypeptide. Such candidate agents or reagents include polypeptides, oligonucleotides, peptidomimetics, carbohydrates or small molecules such as small organic or inorganic molecules (e.g., non-nucleic acid small organic chemical compounds) that modulate expression (protein or mRNA) or activity of one or more glucose transport-related polypeptides or nucleic acids. In general, screening assays involve assaying the effect of a test agent on expression or activity of a glucose transport-related nucleic acid or polypeptide in a test sample (i.e., a sample containing the glucose transport-related nucleic acid or polypeptide). Expression or activity in the presence of the test compound or agent is compared to expression or activity in a control sample (i.e., a sample containing a glucose transport-related polypeptide that was not incubated in the presence of the test compound). A change in the expression or activity of the glucose transport-related nucleic acid or polypeptide in the test sample compared to the control indicates that the test agent or compound modulates expression or activity of the glucose transport-related nucleic acid or polypeptide and is a candidate agent.

In one embodiment, the invention provides assays for screening candidate agents that bind to or modulate the activity of a polypeptide or nucleic acid of the invention or biologically active portion thereof. The compounds to be screened, can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam, 1997, Anticancer Drug Des. 12:145).

Examples of methods for the synthesis of molecular libraries can be found in the art, for example in: De Witt et al., 1993, Proc. Natl. Acad. Sci. USA 90:6909; Erb et al., 1994, Proc. Natl. Acad. Sci. USA 91:11422; Zuckermann et al., 1994, J. Med. Chem. 37:2678; Cho et al., 1993, Science 261:1303; Carrell et al., 1994, Angew. Chem. Int. Ed. Engl. 33:2059; Carell et al., 1994, Angew. Chem. Int. Ed. Engl. 33:2061; and Gallop et al., 1994, J. Med. Chem. 37:1233.

Libraries of compounds may be presented in solution (e.g., Houghten, 1992, Bio/Techniques 13:412-421), or on beads (Lam, 1991, Nature 354:82-84), chips (Fodor, 1993, Nature 364:555-556), bacteria (U.S. Pat. No. 5,223,409), spores (U.S. Pat. Nos. 5,571,698; 5,403,484; and 5,223,409), plasmids (Cull et al., 1992, Proc. Natl. Acaci. Sci. USA 89:1865-1869) or phage (Scott and Smith, 1990, Science 249:386-390; Devlin, 1990, Science 249:404-406; Cwirla et al., 1990, Proc. Natl. Acad. Sci. USA 87:6378-6382; and Felici, 1991, J. Mol. Biol. 222:301-310).

In one embodiment, the assay is a cell-based assay in which a cell expressing a polypeptide of the invention, or a biologically active portion thereof, on the cell surface is contacted with a test compound. The ability of the test compound to bind to the polypeptide is then determined. The cell, for example, can be a yeast cell or a cell of mammalian origin. Determining the ability of the test compound to bind to the polypeptide can be accomplished, for example, by coupling the test compound with a radioisotope or enzymatic label such that binding of the test compound to the polypeptide or biologically active portion thereof can be determined by detecting the labeled compound in a complex. For example, test compounds can be labeled with ²⁵I, ³⁵S, ¹⁴C, or ³H, either directly or indirectly, and the radioisotope detected by direct counting of radioemmission or by scintillation counting. Alternatively. test compounds can be enzymatically labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of a polypeptide of the invention, or a biologically active portion thereof, on the cell surface with a known compound which binds to the polypeptide to form an assay mixture, contacting the assay mixture with a test compound, and determining the ability of the test compound to interact with the polypeptide, wherein determining the ability of the test compound to interact with the polypeptide comprises determining the ability of the test compound to preferentially bind to the polypeptide or a biologically active portion thereof as compared to the known compound.

In another embodiment, an assay is a cell-based assay comprising contacting a cell expressing a membrane-bound form of a polypeptide of the invention, or a biologically active portion thereof, on the cell surface with a test compound and determining the ability of the test compound to modulate (e.g., stimulate or inhibit) the activity of the polypeptide or biologically active portion thereof. Determining the ability of the test compound to modulate the activity of the polypeptide or a biologically active portion thereof can be accomplished, for example, by determining the ability of the polypeptide to bind to or interact with a target molecule.

Determining the ability of a polypeptide or nucleic acid of the invention to bind to or interact with a target molecule can be accomplished by one of the methods described herein for determining direct binding. As used herein, a “target molecule” is a molecule with which a selected polypeptide or nucleic acid (e.g., a polypeptide or nucleic acid of the invention) binds or interacts with in nature, for example, a molecule on the surface of a cell which expresses the selected protein, a molecule on the surface of a second cell, a molecule in the extracellular milieu, a molecule associated with the internal surface of a cell membrane or a cytoplasmic molecule. A target molecule can be a polypeptide or nucleic acid of the invention or some other polypeptide, protein or nucleic acid. For example, a target molecule can be a component of a signal transduction pathway which facilitates transduction of an extracellular signal (e.g., a signal generated by binding of a compound to a polypeptide of the invention) through the cell membrane and into the cell or a second intercellular protein which has catalytic activity or a protein which facilitates the association of downstream signaling molecules with a polypeptide of the invention. Determining the ability of a polypeptide of the invention to bind to or interact with a target molecule can also be accomplished by determining the activity of the target molecule. For example, the activity of the target molecule can be determined by detecting induction of a cellular second messenger of the target (e.g., intracellular Ca²⁺, diacylglycerol, or IP3), detecting catalytic/enzymatic activity of the target on an appropriate substrate, detecting the induction of a reporter gene (e.g., a regulatory element that is responsive to a polypeptide of the invention operably linked to a nucleic acid encoding a detectable marker, e.g., luciferase), or detecting a cellular response, for example, cellular differentiation, or cell proliferation. When the target molecule is a nucleic acid, the compound can be, e.g., a ribozyme or antisense molecule.

In yet another embodiment, an assay of the present invention is a cell-free assay comprising contacting a polypeptide or nucleic acid of the invention, or biologically active portion thereof, with a test compound and determining the ability of the test compound to bind to the polypeptide or biologically active portion thereof. Binding of the test compound to the polypeptide can be determined either directly or indirectly as described above. In one embodiment, the assay includes contacting the polypeptide of the invention or biologically active portion thereof with a known compound which binds the polypeptide to form an assay mixture, contacting the assay mixture with a test compound, and determining the ability of the test compound to interact with the polypeptide (e.g., its ability to compete with binding of the known compound), wherein determining the ability of the test compound to interact with the polypeptide comprises determining the ability of the test compound to preferentially bind to the polypeptide or biologically active portion thereof as compared to the known compound. When the test compound is targeted to a nucleic acid, the binding of the test compound to the nucleic acid can be tested, e.g., by binding, by fragmentation of the nucleic acid (as when the test compound is a ribozyme), or by inhibition of transcription or translation in the presence of the test compound.

In another embodiment, an assay is a cell-free assay comprising contacting a polypeptide of the invention or biologically active portion thereof with a test compound and determining the ability of the test compound to modulate (e.g., stimulate or inhibit) the activity of the polypeptide or biologically active portion thereof. For example, determining the ability of the test compound to modulate the activity of the polypeptide can be accomplished by determining the ability of the polypeptide of the invention to modify the target molecule. Such methods can, alternatively, measure the catalytic/enzymatic activity of the target molecule on an appropriate substrate. In general, modulation of the activity of the polypeptide of the invention or biologically portion thereof is determined by comparing the activity in the absence of the test compound to the activity in the presence of the test compound.

In yet another embodiment, the cell-free assay comprises contacting a polypeptide or nucleic acid of the invention, or biologically active portion thereof, with a known compound which binds to the polypeptide to form an assay mixture, contacting the assay mixture with a test compound, and determining the ability of the test compound to interact with the polypeptide or nucleic acid, wherein determining the ability of the test compound to interact with the polypeptide or nucleic acid comprises determining the ability of the polypeptide or nucleic acid to preferentially bind to or modulate the activity of a target molecule.

The cell-free assays of the present invention are amenable to use of either a soluble form or the membrane-bound form of a polypeptide of the invention. In the case of cell-free assays comprising the membrane-bound form of the polypeptide, it may be desirable to utilize a solubilizing agent such that the membrane-bound form of the polypeptide is maintained in solution. Examples of such solubilizing agents include non-ionic detergents such as n-octylglucoside, n-dodecylglucoside, n-octylmaltoside, octanoyl-N-methylglucamide, decanoyl-N-methylglucamide, Triton X-100, Triton X-114, Thesit, Isotridecypoly(ethylene glycol ether)n, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl-N,N-dimethyl-3-ammonio-1-propane sulfonate.

In more than one embodiment of the above assay methods of the present invention, it may be desirable to immobilize either the polypeptide of the invention or its target molecule to facilitate separation of complexed from uncomplexed forms of one or both of the proteins, as well as to accommodate automation of the assay. Binding of a test compound to the polypeptide, or interaction of the polypeptide with a target molecule in the presence and absence of a test agent, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase fusion proteins or glutathione-S-transferase fusion proteins can be adsorbed onto glutathione sepharose beads (Sigma Chemical; St. Louis, Mo.) or glutathione derivatized microtitre plates, which are then combined with the test compound or the test compound and either the non-adsorbed target protein or a polypeptide of the invention, and the mixture incubated under conditions conducive to complex formation (e.g., at physiological conditions for salt and pH). Following incubation, the beads or microtitre plate wells are washed to remove any unbound components and complex formation is measured either directly or indirectly, for example, as described above. Alternatively, the complexes can be dissociated from the matrix, and the level of binding or activity of the polypeptide of the invention can be determined using standard techniques.

Other techniques for immobilizing proteins on matrices can also be used in the screening assays of the invention. For example, either the polypeptide of the invention or its target molecule can be immobilized utilizing conjugation of biotin and streptavidin. Biotinylated polypeptide of the invention or target molecules can be prepared from biotin-NHS (N-hydroxy-succinimide) using techniques well known in the art (e.g., biotinylation kit, Pierce Chemicals; Rockford, Ill.), and immobilized in the wells of streptavidin-coated 96 well plates (Pierce Chemical). Alternatively, antibodies reactive with the polypeptide of the invention or target molecules but which do not interfere with binding of the polypeptide of the invention to its target molecule can be derivatized to the wells of the plate, and unbound target or polypeptide of the invention trapped in the wells by antibody conjugation. Methods for detecting such complexes such as GST-immobilized complexes, include immunodetection of complexes using antibodies reactive with the polypeptide of the invention or target molecule, as well as enzyme-linked assays which rely on detecting an enzymatic activity associated with the polypeptide of the invention or target molecule.

In another embodiment, modulators of expression of a polypeptide of the invention are identified in a method in which a cell is contacted with a test agent or compound and the expression of the selected mRNA or protein (i.e., the mRNA or protein corresponding to a polypeptide or nucleic acid of the invention) in the cell is determined. The level of expression of the selected mRNA or protein in the presence of the test agent is compared to the level of expression of the selected mRNA or protein in the absence of the test agent. The test agent can then be identified as a modulator of expression of the polypeptide (i.e., a candidate compound)of the invention based on this comparison. For example, when expression of the selected mRNA or protein is greater (statistically significantly greater) in the presence of the test agent than in its absence, the test agent is identified as a candidate agent that is a stimulator of the selected mRNA or protein expression. Alternatively, when expression of the selected mRNA or protein is less (statistically significantly less) in the presence of the test agent than in its absence, the test agent is identified as a candidate agent that is an inhibitor of the selected mRNA or protein expression. The level of the selected mRNA or protein expression in the cells can be determined by methods described herein.

In yet another aspect of the invention, a polypeptide of the inventions can be used as “bait proteins” in a two-hybrid assay or three hybrid assay (see, e.g., U.S. Pat. No. 5,283,317; Zervos et al., 1993, Cell 72:223-232; Madura et al., 1993, J. Biol. Chem. 268:12046-12054; Bartel et al., 1993, Bio/Techniques 14:920-924; Iwabuchi et al., 1993, Oncogene 8:1693-1696; and PCT Publication No. WO 94/10300), to identify other proteins, that bind to or interact with the polypeptide of the invention and modulate activity of the polypeptide of the invention. Such binding proteins are also likely to be involved in the propagation of signals by the polypeptide of the inventions as, for example, upstream or downstream elements of a signaling pathway involving the polypeptide of the invention.

Electronic Data Storage and Processing

The invention includes nucleic acid and polypeptide sequences that are provided in digital form that can be transmitted and read electronically (e.g., in a database). In some embodiments, the database can be queried for comparison with data provided (e.g., a nucleic acid sequence or a pattern of expression). All sequence information or data provided for comparison with the database can be transmitted to the database, e.g., by email, via the Internet, on diskette, or any other mode of electronic or non-electronic communication.

The invention thus features an electronic method of determining whether a patient has a glucose-transport related disorder by obtaining an electronic form of a nucleic acid sequence from the patient; obtaining a database of nucleic acid molecules whose expression is altered in a glucose transport-related disorder such as type II diabetes that includes nucleic acid molecules of individuals with glucose-transport related disorders; and comparing the patient nucleic acid sequence with the nucleic acid molecules in the database, wherein a patient nucleic acid sequence that matches a nucleic acid molecule in the database indicates the patient has or is at risk for a glucose-transport related disorder.

The invention also includes a database that includes an electronic form (e.g., digital form) of the nucleic acid molecules of the invention, and a computer-readable instructions for a processor to carry out the comparison method. The database can also be stored on a machine- or computer-readable medium, and can be accessed, e.g., through a communications network, such as the Internet.

As used herein, “sequence information” refers to any nucleotide and/or amino acid sequence information, including but not limited to full-length nucleotide and/or amino acid sequences, partial nucleotide and/or amino acid sequences. Moreover, information “related to” the sequence information includes detecting the presence or absence of a sequence (e.g., detection of expression of a sequence, fragment, or polymorphism), determination of the level of a sequence (e.g., detection of a level of expression, for example, a quantitative detection), detection of a reactivity to a sequence (e.g., detection of protein expression and/or levels, for example, using a sequence-specific antibody), detection of a pattern of expression of two or more sequences, and the like. These sequences can be read by electronic apparatus and can be stored on any suitable medium for storing, holding, or containing data or information that can be read and accessed by an electronic apparatus. Such media can include, but are not limited to: magnetic storage media, such as floppy disks, hard disk storage medium, and magnetic tape; optical storage media such as compact disks; electronic storage media such as RAM, ROM, EPROM, EEPROM and the like; general hard disks and hybrids of these categories such as magnetic/optical storage media. The medium is adapted or configured for having recorded thereon sequence information.

As used herein, the term “electronic apparatus” is intended to include any suitable computing or processing apparatus or other device configured or adapted for storing data or information. Examples of electronic apparatus suitable for use with the present invention include stand-alone computing apparatus such as personal computers (PCs) and large computer systems. These systems can be accessed by communications networks, including local area networks (LAN), wide area networks (WAN), Internet, Intranet, and Extranet. For example, the database can be made available on an Internet website.

As used herein, “stored” refers to a process for encoding information on the electronic apparatus readable medium. Those skilled in the art can readily adopt any of the presently known methods for recording information on known media to generate manufactures comprising the sequence information.

A variety of software programs and formats can be used to store the sequence information on the electronic apparatus readable medium. For example, the sequence information can be represented in a word processing text file, formatted in commercially-available software such as WordPerfect® and MicroSoft® Word®, or represented in the form of an ASCII file, stored in a database application, such as DB2®, Sybase®, Oracle®, or the like, as well as in other forms. Any number of data processor structuring formats (e.g., text file or database) can be employed to obtain or create a medium having recorded thereon the sequence information.

By providing sequence information in machine or computer-readable form, one can routinely access the sequence information for a variety of purposes. For example, one skilled in the art can use the sequence information in computer-readable form to compare a specific sequence with the sequence information stored within a database. Search means are used to identify fragments or regions of the sequences that match a particular sequence.

The present invention therefore provides a medium for storing or holding a database or instructions for performing a method for determining whether an individual has a specific disease or disorder related to glucose transport or a pre-disposition for a specific disease or disorder related to glucose transport, wherein the method can include analyzing the individual's sequence information and based on the sequence information, determining whether the individual has a particular disorder or a predisposition for a particular disorder associated with a specific genetic sequence, and/or recommending a particular treatment for the disorder or pre-disorder condition. For example, the pattern of expression of glucose transport-related sequences or proteins from an individual suspected of having a glucose transport-related disorder (e.g., type II diabetes) can be analyzed, and, based on the analysis (e.g., aberrant expression of one or more glucose transport-related genes), a diagnosis provided and instructions for treatment.

The invention will be further described in the following examples which do not limit the scope of the invention described in the claims.

### EXAMPLES

Three approaches were used to identify genes and proteins involved in glucose transport. First, several subtractive cDNA libraries were constructed that consist of genes selectively expressed in insulin-responsive tissues. Furthermore, it has been discovered that at least two of these genes have a role in regulating GLUT4 translocation. As a second approach, microarrays were screened with fluorescently labeled probes synthesized from mRNA isolated from insulin-responsive tissues. In the third approach, a subcellular fraction was prepared that was enriched for vesicles involved in glucose transport. Proteins from this fraction were prepared and analyzed using microsequencing techniques. Additional analysis comparing the predicted protein sequences obtained in the first two approaches with the vesicle protein sequences provided a subset of sequences involved in glucose transport that are useful for certain aspects of the invention.

### Example 1

Subtractive Libraries

Two methods were used to construct subtractive libraries.

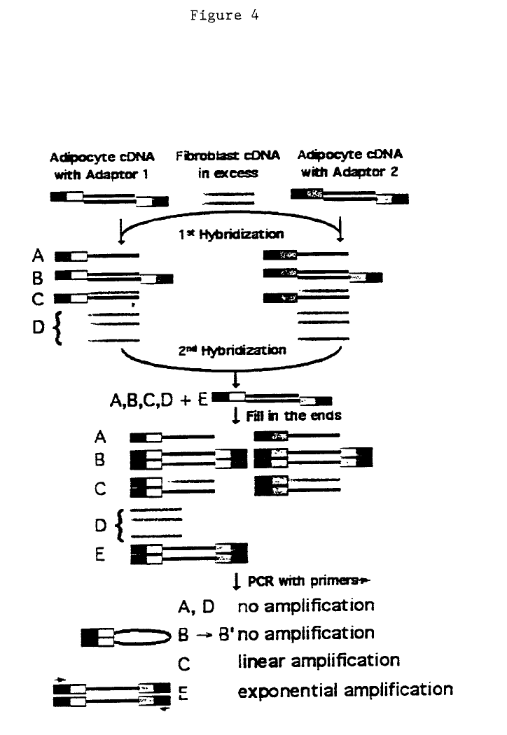

In the first method, suppression subtractive hybridization was used (Diatchenko et al., 1996, Proc Natl. Acad. Sci USA 93:6025-30). In this method, a first library was constructed that consisted of sequences that are highly expressed in muscle, but not in 3T3-L1 fibroblasts (available from American Type Culture Collection; ATCC). The second library consisted of sequences that are highly expressed in 3T3-L1 adipocytes, but not in 3T3-L1 fibroblasts. The general method for this procedure is diagrammed in FIG. 4.

Libraries were constructed by reverse transcription of total mRNA isolated from plates of confluent 3T3-L1 fibroblasts and 3T3-L1 adipocytes 9 to 10 days after the start of differentiation. The resulting cDNAs were then digested with the restriction enzyme Rsa I. Digested adipocyte cDNA was divided into two pools, and each pool was ligated to a different oligonucleotide adaptor. Adaptor 1 was:

| | |
|---|---|
| 5′-CTAATACGACTCACTATAGGGCTCGAGCGGCCGCCCGGGCAGGT-3′ | (SEQ ID NO: 94) |
| GGCCCGTCCA-5′ | (SEQ ID NO: 95) |
| Adaptor 2 was: | |
| 5′-CTAATACGACTCACTATAGGGCAGCGTGGTCGCGGCCGAGGT-3′ | (SEQ ID NO: 96) |
| GCCGGCTCCA-5′ | (SEQ ID NO: 97) |

Each pool of adipocyte cDNA (tester DNA) was then hybridized with an excess of fibroblast cDNA (driver DNA) for 9 hours at 68° C. The two hybridization mixtures were combined and incubated overnight at 68° C. After hybridization the 5′ overhangs were filled in with Taq DNA polymerase, and amplified by PCR using primers that are homologous to each of the adaptors. This subtraction procedure was also performed using the mouse muscle cDNA as the tester, and 3T3-L1 fibroblast cDNA as the driver.

As a test to demonstrate that muscle and adipocyte specific transcripts are amplified by this procedure, the final products of both subtractions were amplified using PCR primers internal to GLUT4 and α-tubulin transcripts. The final product of muscle subtraction (SUB) and the unsubtracted muscle cDNA (UNSUB) were used for PCR analysis with primers internal to the coding regions of Glut4 (G4) and α-tubulin-1 (TUB). Glut4 and α-tubulin-1 primers were designed to amplify 485 bp and 408 bp fragments respectively. PCR samples were removed after 23, 28 and 33 PCR cycles and loaded onto a 1.5% TAE (40 mM Tris-acetate, pH8.0 1 mM EDTA) agarose gel. The gel was stained with ethidium bromide and visualized with WV light. As expected, GLUT4 cDNA (representing GLUT4 expression) was found in the subtracted muscle cDNA but tubulin cDNA was present in relatively small amounts because tubulin is expressed in both fibroblasts and muscle (and so a substantial amount of the tubulin sequence was subtracted out). GLUT4 is expressed in muscle but not in fibroblasts, and so, as expected present in relatively large amounts. In the muscle-subtracted cDNA, the GLUT4 signal is stronger in earlier PCR cycles, while the tubulin signal in suppressed. Similar results were obtained with PCR analysis with 3T3-L1 adipocyte-subtracted cDNA.

To construct the libraries, the final PCR products from the 3T3-L1 adipocyte subtraction were digested with Rsa I and cloned into Eco RV restricted the pBluescript SK+ vector (STRATAGENE®) creating a library of adipocyte subtractive clones. The library contained approximately 2×10³ clones. The cloned plasmid DNA sequences were analyzed by dideoxy sequencing with either the M13-20 or reverse primer on an ABI 377 automatic sequencer. In an initial round of sequencing, 183 independent clones, representing expression from 65 different genes, were sequenced. Sequences were analyzed in a search against the non-redundant (NR) nucleotide database using the Blast program at www.ncbi.nlm.nih.gov/blast/blast.cgi. The gapped BLAST program was used against the non-redundant or the dbest database. All BLAST searches were performed using the default settings which are: Expect=10; Filter for Low complexity: on; Filter for Human Repeats: off; Mask for lookup table only: off; Matrix=Blosum62; Gap existence cost=11; Per residue gap cost=1; Lambda ratio=85.

Genes previously shown to be preferentially expressed or not preferentially expressed in adipocytes are those in which their mRNA expression profiles have been published in journal articles in the Medline database. A summary of these sequences is shown in FIGS. 6A-6E. Approximately 60% of the sequenced clones in this library were from genes previously reported as overexpressed in 3T3-L1 adipocytes. Another 23% of the clones consisted of known gene sequences whose expression pattern was known in adipocytes, while 13% of the sequenced clones had unknown (previously unreported) sequences. Four percent of the cloned sequences are from genes of mitochondrial origin. The identity of the genes in the subtractive library that have already shown to be preferentially expressed in 3T3-L1 adipocytes are listed in FIGS. 6A-6E. Genes, such as adipoQ and stearoyl-CoA desaturase, that are found at the highest frequency in this subtractive library are also those that were discovered in previous attempts to clone genes that are highly expressed in 3T3-L1 adipocytes upon differentiation (Ntambi et al., 1988, J. Biol. Chem 263:17291-17300; Bernlohr et al., 1984, Proc. Nat. Acad. Sci. USA 81: 5468-5472; Hu et al., 1996, J. Biol.

Chem. 271:10697-10703; Min and Spiegelman, 1986, Nucleic Acids Res. 14:8879-8892).

Sequences that are expressed in the Adipocyte Subtractive library that are from genes with unknown function are listed in FIGS. 3A-3E.

### Example 2

Construction of a Muscle-adipocyte Library

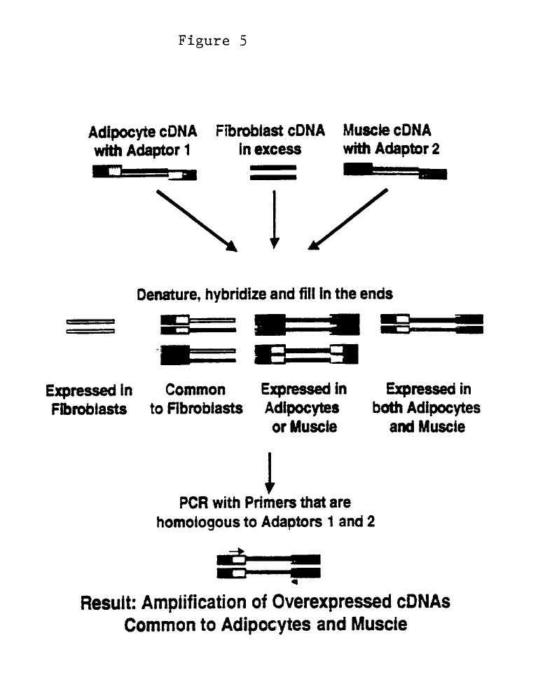

To identify genes encoding proteins that are involved in glucose transport, gene expression in 3T3-L1 adipocytes and muscle was investigated. To accomplish this, another library was constructed consisting of genes that fulfilled the following two criteria. First, the genes had to be highly expressed in both 3T3-L1 adipocytes and mouse skeletal muscle; second, the genes could not be highly expressed in 3T3-L1 fibroblasts. This library, the Muscle-Adipocyte Union Library (MAU library), was constructed using a modification of the suppression subtractive hybridization technique (FIG. 5). The method was like the subtractive suppression modification technique described in FIG. 4 except that adaptor 1 was ligated to Rsa I-digested 3T3-L1 adipocyte cDNA while adaptor 2 was ligated to Rsa I-digested mouse muscle cDNA. Both cDNAs were then hybridized to an excess of 3T3-L1 fibroblast DNA. The two hybridization reactions were then mixed to create hybrid molecules in which one strand originated from adipocytes and the second strand of the hybrid was from muscle. Because only these hybrid molecules have different adaptors on each end, they can be PCR amplified, unlike the rest of the cDNAs. These hybrid products were then amplified using PCR. The final PCR products of the 3T3-L1 muscle-adipocyte union subtraction were cloned into overhang vector pCR2.1. (INVITROGEN®) to produce a library of approximately 10⁴ clones. Plasmid DNAs were dideoxy sequenced with the either the M13-20 or reverse primer on an ABI 377 automatic sequencer. Sequences were searched against the non-redundant (NR) nucleotide database using the Blast program at www.ncbi.nlm.nih.gov/blast/blast.cgi. Genes previously shown to be overexpressed or not overexpressed in adipocytes are those in which their mRNA expression profiles has been published in journal articles in the Medline database. FIGS. 7A-7U show the summary of sequences from this library. These clones represent as many as 265 different genes. About 40% of these sequences are expressed from genes that have previously been shown to be preferentially in muscle, adipocytes, or both tissues. Another 26% of the clones are sequences from known genes whose expression profile is not known, and 17% of the clones represent previously unidentified genes. A large percentage of sequences (12%) represent genes of mitochondrial origin. FIG. 1 shows sequences from this library that are novel, and FIGS. 2A-2R show the sequences of selected clones from this library. FIGS. 7A-7U show the genes that encode the sequences identified in the MAU library including the GenBank accession no., when one is known. FIGS. 7A-7U also list the homologous human genes for these sequences and the expression profile of each sequence with respect to its expression in adipocytes and muscle.

The sequences identified in this manner are useful, e.g., for detecting a glucose transport-related disorder such as type II diabetes.

### Example 3

mRNA Expression Profiles of Unknown Genes in the 3T3-L1 Adipocyte Subtractive and the Muscle-adipocyte Union Libraries.

To determine the expression profile of cognate RNA from library clones that have not been previously reported to be overexpressed (i.e., preferentially expressed) in insulin-sensitive (e.g., adipocyte and muscle) tissues, expression of these sequences was analyzed in undifferentiated 3T3-L1 cells and differentiated 3T3-L1 adipocytes. Northern blot analysis was used in which 3T3-L1 and mouse multi-tissue Northern blots were probed.

Cloned inserts from the Adipocyte Subtractive Library clones were labeled with ³²P-dCTP and used in an initial screen to probe Northern blots of total RNA from 3T3-L1 fibroblasts and adipocytes. For Northern blotting, 3T3-L1 and multiple tissue total RNA (10 μg) were electrophoresed on 1.2% agarose/6.6% formaldehyde gels, then transferred to Nytran membranes. Before transfer, gels were stained with ethidium bromide and visualized with UV light in order to confirm equal loading of RNAs. Blots were probed with inserts containing fragments of previously unidentified genes from both libraries Probes were labeled with P³²-dCTP and incubated with the membranes overnight at 42° C. Blots were washed twice with 2×SSC/0.1% SDS at room temperature, twice in 0.2×SSC/0.1% SDS at room temperature and twice in 0.2×SSC/0.1% SDS at 42° C. After washing, blots were exposed to a phosphor screen for one to three days. Phosphor screens were scanned with the Storm 860 Scanner from Molecular Dynamics. Full-length clones for many of these unknown genes have been obtained either by purchasing IMAGE Consortium clones or by screening muscle or adipocyte lambda libraries (such libraries can be made using methods known in the art).

Seventy-eight clones from the Adipocyte Subtractive Library were characterized. Sixty of the 78 cloned sequences (approximately 75%) were preferentially expressed upon adipocyte differentiation (i.e., in 3T3-L1 adipocytes).

Thirty-two clones from the 3T3-L1 Muscle-Adipocyte Union library (MAU library) were analyzed. Nineteen were preferentially expressed in 3T3-L1 adipocytes. This leads to the conclusion that approximately 50% of the clones in the MAU library, whose expression has not previously been reported, are preferentially expressed in 3T3-L1 adipocytes. This indicates that approximately 80% of the clones in the 3T3-L1 Adipocyte Subtractive Library and 70% of the clones in the Muscle-Adipocyte Union Library (MAU library) are highly expressed in at least one insulin-sensitive tissue. (For the 3T3-L1 Adipocyte Subtractive Library, 60% of sequences previously shown to be preferentially expressed +½ of 40%=80%; for MAU library, 40% of sequences previously shown to be preferentially expressed +½ of 60% of uncharacterized genes=70%). Genes that were found to be preferentially expressed in 3T3-L1 adipocytes were used to probe mouse multi-tissue Northern blots. Using Northern analysis, it was confirmed that 11 previously unidentified genes from the MAU library (i.e., genes expressed in adipocytes and muscle) are expressed in at least two different insulin-sensitive tissues (see FIGS. 6A-6E and 7A-7U; “overexpressed” indicates that the sequence was found to be preferentially expressed in insulin-sensitive cells in these experiments).

Using multi-tissue Northern blots it was shown that that six previously identified genes are highly expressed in insulin-sensitive tissues. Furthermore, at least two of these proteins have a role in regulating GLUT4. This was determined as follows. Three clones in the Muscle-Adipocyte Union Library consist of the 3′ end of PP2Cα1 (Genbank Acession No. D28117 Kato et al., 1994, Gene 145:311-312). Northern blot analysis demonstrated that at least three transcripts of PP2Cα are highly expressed in both 3T3-L1 adipocytes and in mouse fat. We further examined mRNA expression of PP2Cα. For Northern blotting, 3T3-L1 and multiple tissue total RNA (10 μg) were separated by electrophoresis on 1.2% agarose/6.6% formaldehyde gels, then transferred on to Nytran membranes. Blots were probed with library clone c0452, which contains the last 216 base pairs of the PP2Cα1 coding sequence along with the 288 base pairs of 3′ noncoding region. Probes were labeled with P³²-dCTP and incubated with the membranes overnight at 42° C. Blots were washed twice with 2×SSC/0.1% SDS at room temperature, twice in 0.2×SSC/0.1% SDS at room temperature and twice in 0.2×SSC/0.1% SDS at 42° C. After washing, 3T3-L1 blots were exposed to film for one day, while multi tissue northern blots were exposed to a phosphor screen for one to three days. Phosphor screens were scanned with the Storm 860 Scanner from Molecular Dynamics.

To assess the role of PP2Cα1 in insulin-stimulated glucose transport, PP2Cα1 protein was microinjected into 3T3-L1 adipocytes, and GLUT4 translocation was determined by immunofluorescence. Microinjection of PP2Cα1 was found to potentiate the ability of a submaximal 1 nM concentration of insulin to translocate GLUT4 to the plasma membrane to levels close if not equal to that of a maximal 10 nM insulin stimulation. To examine the effect of microinjected PP2Cα1 on GLUT4 translocation, 3T3-L1 adipocytes were incubated in serum free medium for two hours and microinjected with either IgG alone or PP2Cα along with IgG. Sixty minutes later adipocytes were incubated with media alone, 1 nM insulin or a maximally effective concentration of insulin (10 nM) for 30 minutes. Cells were then fixed with methanol and then stained with anti-GLUT4 antibody. Adipocytes were examined using fluorescence microscopy (Zeiss Axioskop, at 630×magnification) and scored for scored for the presence of substantial cell surface GLUT4 immunoreactivity at the plasma membrane. Controls are cells on the same coverslips that were not injected. Microinjection of phosphatases 2A or 2B had no effect on the ability of insulin to activate GLUT4 translocation. Western blotting has also revealed that PP2Cα selectively co-immunoprecipitates insulin receptors but not PDGF receptors in an insulin-enhanced manner.

Gα11 (Q209L) Induced 2-Deoxyglucose Uptake in Differentiated 3T3-L1 Adipocytes.

Gα11 sequence (Genbank Accession No. U37411; Davignon et al., 1996, Genomics 31:359-366) was identified in the 3T3-L1 Adipocyte Subtractive Library. This protein is a member of the Gaq family which are heterotimeric components of G protein complexes. Northern blot analysis confirmed that Gα11 expression is induced upon 3T3-L1 adipocyte differentiation, and that it is more abundant by far in fat than in any other tissue. Differentiated 3T3-L1 adipocytes were seeded at 150,000 cells per well in 24 well plates and then infected with either control or Gα11 (Q209L) adenoviruses. Thirty hours after infection, plates were serum starved for two hours in Krebs-Ringer phosphate buffer with BSA and pyruvate. Plates were then treated with or without wortmannin (a specific inhibitor of P13 kinase) for 15 minutes followed by stimulation with insulin or endothelin for 30 minutes. Cells were then assayed for 2-deoxyglucose uptake as described in Frost and Lane (1985, J. Biol. Chem. 260:2646-2652).

For Northern blotting, 3T3-L1 and multiple tissue total RNA (10 μg) were separated on 1.2% agarose/6.6% formaldehyde gels, then transferred on to Nytran membranes. Blots were probed with library clone b0031, which contains nt 237 to nt 435 of the Gα11 coding sequence. Probes were labeled with P³²-dCTP and incubated with the membranes overnight at 42° C. Blots were washed twice with 2×SSC/0.1% SDS at room temperature, twice in 0.2×SSC/0.1% SDS at room temperature and twice in 0.2×SSC/0.1% SDS at 42° C. After washing, 3T3-L1 blots were exposed to film for one day, while multi tissue northern blots were exposed to a phosphor screen for three days. Phosphor screens were scanned with the Storm 860 Scanner from Molecular Dynamics. A closely related protein Gq did not have this expression profile. Infection of 3T3-L1 adipocytes with a recombinant adenovirus expressing a constitutively active form of Gα11 expression, but not the native protein led to an increase in GLUT4 concentration in the plasma membrane, and a fourfold increase in glucose uptake in a wortmannin-insensitive manner. Thus, wortmannin does not inhibit the ability of the active form of Gα11 to stimulate GLUT4 translocation.

Since PI3 kinase activation is required for insulin to activate GLUT4 translocation, these data indicate that Gaα11 is likely a mediator of PI3 kinase independent activators of GLUT4 translocation, such as endothelin. In addition, these data demonstrate that glucose transport-related genes were identified using the methods described herein. They also illustrate an assay for identifying glucose transport-related sequences that are PI3 kinase independent activators of GLUT4 translocation.

### Example 4

Polypeptides Isolated from GLUT4-Enriched Vesicles

The GLUT4 glucose transporter resides primarily in perinuclear membranes in unstimulated 3T3-L1 adipocytes and is acutely translocated to the cell surface in response to insulin. A novel method of purifying intracellular GLUT4-enriched membranes was used to identify polypeptides involved in glucose transport.

Antibodies

Rabbit polyclonal anti-GLUT4 antibody was raised against the C-terminal 12 amino acid sequence of GLUT4. Mouse anti-transferrin receptor was from Zymed. Rabbit polyclonal anti-VAMP2 antibody was from StressGen Biotechnologies Corp. Mouse monoclonal anti-vimentin antibody used in immunoblots and immuno-electron microscopy analysis was from Santa Cruz. Mouse monoclonal anti-α-tubulin antibody, used in immunoblot and immuno-electron microscopy analysis and the secondary antibodies conjugated to gold particles for immuno-electron microscopy were from Amersham Pharmacia Biotech.

Immunoblotting

Fractions from velocity gradients and equilibrium density gradient were prepared as described above and aliquots from these fractions were subjected to SDS-PAGE on resolving gels according to Laemmli (1970, Nature 227:680-685). Separated proteins were electrophoretically transferred to nitrocellulose membrane, blocked with 3% nonfat milk and 1% BSA in TTBS (0.05% Tween 20 in Tris-buffered saline) and then incubated with primary antibody in TTBS containing 1% BSA. After incubation, membranes were washed with TTBS and incubated with horseradish peroxidase-labeled anti-mouse IgG for the detection of monoclonal antibodies or with horseradish peroxidase-labeled anti-rabbit IgG for detection of polyclonal antibodies. Proteins were visualized using an enhanced chemiluminescent substrate kit (Amersham Pharmacia Biotech) and immunoblot intensities were quantified by a scanning densitometer.

Electron Microscopy

GLUT4-containing membranes of the insulin sensitive fractions from the equilibrium density gradient were isolated as described above. Fractions were pooled, pelleted by centrifugation at 48,000 rpm for 2 hours, resuspended in PBS and fixed in a final concentration of 2% paraformaldehyde in PBS. GLUT4-vesicles were then adsorbed to Formvard-coated gold grids and processed for double labeling as outlined in Martin et al. (supra) and Sleeman et al. (1998, J. Biol. Chem. 273:3132-3135). Grids were incubated with 50 μl of primary antibody diluted in 1% BSA and PBS as follows: anti-GLUT4, anti-IRAP, anti-vimentin, anti-α-tubulin or non-immune IgG, as a negative control. After incubation with each IgG fraction, grids were labeled with either 5 or 15 nm gold particles conjugated to the secondary antibody (goat anti-rabbit or goat anti-mouse). Grids were stained with 1% uranyl acetate, dried and viewed using a transmission electron microscope PHILLIPS CM.10.

Purification of Insulin-responsive GLUT4-containing Membranes

GLUT4-containing membranes were prepared by first isolating low density (LD) microsomes then subjecting these to further purification on sucrose velocity gradients. Finally, the GLUT4 fractions from the sucrose gradients were subjected to equilibrium density sucrose gradients. The preparations were made from primary, unstimulated or insulin stimulated rat adipocytes, although the could also be prepared from other tissues, e.g., striatal muscle.

To prepare the initial crude membrane preparations for purification, adipocytes were isolated from epididymal fat pads of Male Sprague-Dawley Rats (125-150 g) by collagenase digestion in Krebs-RingerdHEPES, pH 7.4, supplemented with 2% bovine serum albumin and 2 mM pyruvate. Following digestion, the cells were washed and permitted to recover for 30 minutes. The cells were then incubated at 37° C. with or without 100 nM insulin for 20 minutes. The cells were washed with PBS and immediately homogenized in buffer A (50 mM HEPES, pH 7.4, 10 mM NaF, 1 mM NaPPi, 0.1 mM Na₃VO₄, 1 mM phenylmethylsulfonyl fluoride, 10 μg/ml aprotinin, and 10 μg/ml leupeptin), and then subjected to differential centrifugation as described in Czech and Buxton, 1993, J. Biol. Chem. 268:9187-9190. Low density microsomes were prepared by modifications of previously described methods (Mackeell, D. W. and Jarret, L., 1970, J. Cell Biol., 44:417432). Briefly, cells were homogenized for 15 strokes with a motor-driven Teflon/glass homogenizer in 24 ml of buffer containing 10 mM Tris-Cl, pH 7.4, 1 mM EDTA, 250 mM sucrose, 10 mM NaF, 1 mM phenylmethylsufonyl fluoride. The homogenate were brought to 4° C. and centrifuged for 20 minutes at 16,000×g. The 16,000×g supernatant was centrifuged at 48,000×g for 20 minutes to obtain a pellet of high density microsomes and the resulting supernatant was centrifuged for 90 minutes at 200,000×g to obtain a pellet of low density microsomes. The low density microsomes were resuspended at a final concentration of approximately 1-3 mg/ml. Protein was quantified using the bicinchoninic acid protein determination kit (Pierce) with bovine serum albumin as standard.

GLUT4-enriched fractions were then isolated from LD microsomal fractions utilizing the sedimentation sucrose velocity gradient centrifugation (Kandror et al., 1995, Biochem. J. 307:383-390; Heller-Harrision,et al., 1996, J. Biol. Chem. 271:10200-10204). Briefly, 1.5 to 2 mg of LD microsomal fractions were loaded onto a 10-35% sucrose velocity gradient (sucrose in buffer B: 20 mM HEPES, pH 7.4, 100 mM NaCl, 1 mM EDTA, 2 mM dithiothreitol, 1 mM, 10 mM NaF, 1 mM NaPPi, 0.1 mM Na₃VO₄, 1 mM phenylmethylsulfonyl fluoride, 10 μg/ml aprotinin and 10 μg/ml leupeptin) and centrifuged for 3.5 hours at 110,000×g rpm in an SW28 rotor (Beckman) and 1 ml fractions were collected. The crude membrane fraction contains most of the GLUT4 present in unstimulated adipocytes and is composed primarily of intracellular membranes (Czech and Buxton, supra). This additional centrifugation step separates about 90% of the total membrane protein (fractions 1-7) from the GLUT4-enriched membranes (fractions 8-18).

Insulin treatment of rat adipocytes prior to disruption of the cells and preparation of these membranes causes a marked decrease in the yield of GLUT4 present in the latter fractions. However, no such insulin effect is observed when total membrane protein is measured because these membranes are still highly contaminated with membranes that do not contain GLUT4 and are not insulin-responsive.

To further resolve the membrane species associated with GLUT4, fractions 8-18 which contained most of the GLUT4 from the sucrose velocity gradient were subjected to equilibrium gradient centrifugation. Fractions from sucrose velocity gradients containing GLUT4-membranes (Fractions 8 to 18) were pooled, pelleted by ultracentrifugation at 48,000 rpm for 1.5 hours, resuspended in buffer B and then loaded onto an equilibrium density sucrose gradient (10-65% (w/v) in buffer B and centrifuged at 150,000×g rpm for 18 hours in a SW 50.1 rotor (Beckman). After centrifugation, 0.25 ml fractions were collected starting from the top of the gradient. Fractions were analyzed for the total protein content using a Bradford assay (Bio-Rad).

Most of the membrane protein was distributed over fractions 5-20 after this procedure, whereas most of the GLUT4 was distributed within fractions 7-14. Importantly, GLUT4 was localized into two types of membranes (GLUT4 membranes) that can be distinguished based on their sensitivity to insulin. The amount of GLUT4 in fractions 7-9 (peak 1) was decreased when the cells were treated with insulin before homogenization and preparation of membranes, whereas the GLUT4 in fractions 10-20 (peak 2) was not affected by insulin treatment of the adipocytes. Strikingly, measurement of total membrane protein in the fractions of this gradient revealed a similar profile: about a 50% reduction in fractions 7-9 due to insulin action, with no insulin effect observed in fractions 10-20. This observed insulin-mediated decrease in total membranes recovered in fractions 7-9 indicates the successful partial purification of membranes of the insulin-responsive compartment or compartments in primary adipocytes. Similar data were obtained using 3T3-L1 adipocytes.

These methods can be used to, e.g., provide an enriched preparation of glucose transport-related sequences. In addition, in screening assays, a test compound can be incubated with the cells before isolation of the vesicles and the ability of the test compound to affect the localization of the glucose transport-related sequence determined.

### Example 5

Characterization of GLUT4 Membranes

Two additional approaches were used to characterize the membranes resolved by equilibrium gradient centrifugation. First, each fraction from the gradient was analyzed by SDS-PAGE and silver staining of the constituent proteins. This analysis revealed that most of the membrane proteins in fractions 7 and 8 were dramatically reduced when membranes were derived from insulin-treated adipocytes. Certain proteins in fractions 6 and 9 showed the same effect, whereas many did not. These results suggest that membranes resolved in fractions 7 and 8 are highly purified insulin-responsive membranes, while those in fractions 6 and 9 are only partially purified. Membranes in higher density fractions show no detectable insulin-sensitivity in spite of the presence of significant GLUT4 protein. Many of the protein bands in the insulin-sensitive membranes are also present in the membranes that are not responsive to the hormone. These data are consistent with the hypothesis that the insulin sensitive membranes containing GLUT4 contain many of the same constituent proteins as other cell membranes that function in a hormone-insensitive mode. Thus, these proteins may also be targets for drugs that potentiate insulin action and ameliorate type II diabetes.

To further characterize the GLUT4 membrane preparation, we determined the distribution of transferrin receptors, thought to be present in endosomal membranes, and VAMP2 (vesicle-associated membrane protein), thought to be associated with insulin-sensitive GLUT4-containing membranes (Kandror and Pilch, 1996, J. Biol. Chem. 271:21703-21708; Kandror and Pilch, 1996, Am. J. Physiol. 271:E1-E14). Surprisingly, both of these proteins were present in the fractions that were responsive to insulin and their distributions were more restricted to these fractions than was GLUT4 itself. These data suggest that the insulin-sensitive membranes in these fractions are contaminated by recycling endosomes, that transferrin receptor is present in the insulin-sensitive membranes, or both. The restriction of VAMP2 to the insulin-sensitive fractions is consistent with data showing that VAMP2 function is necessary for GLUT4 translocation to the plasma membrane in response to insulin (Cain et al., 1992, J. Biol. Chem. 267:11681-11684; Martin et al., 1996, J. Cell. Biol. 134:625-635).

Expression of transferrin and/or VAMP2 can therefore be used as part of a system analyzing glucose transport, e.g., in diagnosing type II diabetes.

These experiments provide an example of a method for analyzing glucose transport, e.g., in an individual with type II diabetes. In such a case, insulin-sensitive cells from the individual are cultured and analyzed as above. Alterations in the amount or distribution of vesicle proteins compared to a control (i.e., normal with respect to diabetes) indicate that the individual has or is at-risk for a disorder involving glucose transport. Testing cells from the individual that were cultured in the presence or absence of insulin provides additional information regarding hormone sensitivity (e.g., by examining the distribution of vesicle proteins in the presence and absence of hormone.

### Example 6

Identification of Cytoskeletal Proteins in GLUT4-containing Membranes

To identify proteins present in the insulin-sensitive membranes containing GLUT4, the equivalent of fractions 7 and 8 were pooled, analyzed by SDS-PAGE and the gels silver stained. These results confirmed that many of the resident proteins in the membranes derived from insulin-treated cells were present at lower abundance compared to controls. Many of the protein bands, combined from both lanes, were subjected to tryptic hydrolysis and the peptides analyzed by mass spectrometry as described in Example 6. Of the proteins identified by this procedure, peptides derived from GLUT4 itself appeared in two closely spaced bands. Remarkably, the lower of these bands also contained a peptide corresponding to the phosphorylated form of the COOH-terminus of GLUT4, indicating significant amounts of phosphorylated GLUT4 are present in insulin-sensitive membranes. In addition, peptides corresponding to several proteins previously reported to be present in these membranes were identified, including the IGF-II/mannose-6-phosphate receptor, IRAP (insulin-regulated aminopeptidase), amine oxidase, long chain acyl-CoA synthetase, and SCAMPs (secretory carrier-associated membrane proteins). Two proteins not previously known to be present in insulin-sensitive GLUT4-containing membranes were also identified—vimentin, an intermediate filament subunit, and (x-tubulin, the microtubule protein.

Two approaches were taken to determine if vimentin and α-tubulin are directly associated with membrane vesicles that also contain GLUT4 and are insulin-sensitive. In one approach, the membrane preparations obtained from the equilibrium gradient centrifugation were analyzed by MALDI-TOF MS analysis. In a second approach, the fractions were analyzed using immunoelectron microscopy using anti-GLUT4, anti-vimentin and anti-tubulin antibodies.

MALDI-TOF MS Analysis

Proteins resolved by SDS-PAGE were visualized by silver staining (Bio-Rad) and the bands were excised from one single dimensional 5-15% gel. The silver stained proteins bands were destained and tryptically digested (trypsin) in gel according to Gharahdaghi et al. (1999, Electrophoresis 20:601-605) with some slight modifications. The digested samples were further concentrated and desalted with Millipore Zip Tip C18 micro tips prior to MALDI-TOF (matrix-assisted laser desorption ionization time-of-flight) analysis. MALDI-TOF analyses were performed on a Kratos Analytical Kompact SEQ Instrument, equipped with a curved field reflectron. Peptide masses were searched against the non-redundant protein database using MS-Fit of the Protein Prospector program developed by Clauser et al (1999, Anal. Chem. 71:2871-2882) at University of California, San Francisco. Fragmentation information obtained from individual peptides via Post-Source-Decay (PDS) analysis was searched against the non-redundant protein database using the protein prospector program MS-Tag.

Immunoelectron Microscopy

Standard techniques were used to stain the prepared vesicles with anti-Glut4, anti-vimentin, and anti-tubulin antibodies conjugated to colloidal gold particles. Most of the vesicles in the preparations show reactivity with anti-GLUT4 indicating relatively low contamination with membranes that do not contain the transporter. Anti-vimentin and anti-tubulin antibodies were used to detect vimentin and tubulin in GLUT4-positive membranes. A fraction of these GLUT4-positive membrane vesicles also directly react with anti-vimentin and anti-tubulin. Non-immune antibodies showed no detectable staining of these membranes under the conditions of these experiments, while anti-GLUT4 staining was readily detected. These results indicate that some GLUT4-containing membrane vesicles are associated with the cytoskeletal proteins vimentin, α-tubulin, or both.

To further assess association of vimentin and α-tubulin with insulin-sensitive membranes, the abundance of these cytoskeletal proteins was estimated using Western analysis in each of the membrane fractions obtained by equilibrium gradient centrifugation. The relative abundance of GLUT4 protein versus vimentin and α-tubulin throughout these fractions was analyzed. Both vimentin and alpha-tubulin are present in all of the membrane fractions of the gradient except for the top few fractions. Strikingly, both of these proteins are greatly reduced in abundance in the same gradient fractions in which GLUT4 is reduced in response to the action of insulin. In membrane fractions of higher density, the concentrations of GLUT4, vimentin, and α-tubulin are all unaffected by prior treatment of cells with insulin. Taken together, these experiments demonstrate that two cytoskeletal proteins, vimentin and α-tubulin, are bound to subpopulations of the GLUT4-containing membranes that are insulin-responsive in rat adipocytes.

### Example 7

Identification of Proteins Expressed in GLUT4-containing Vesicles

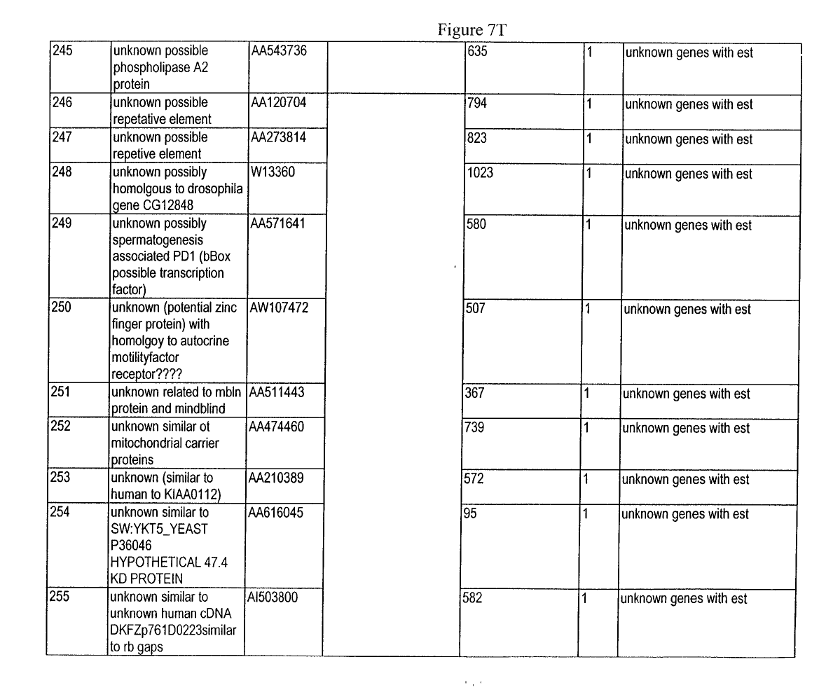

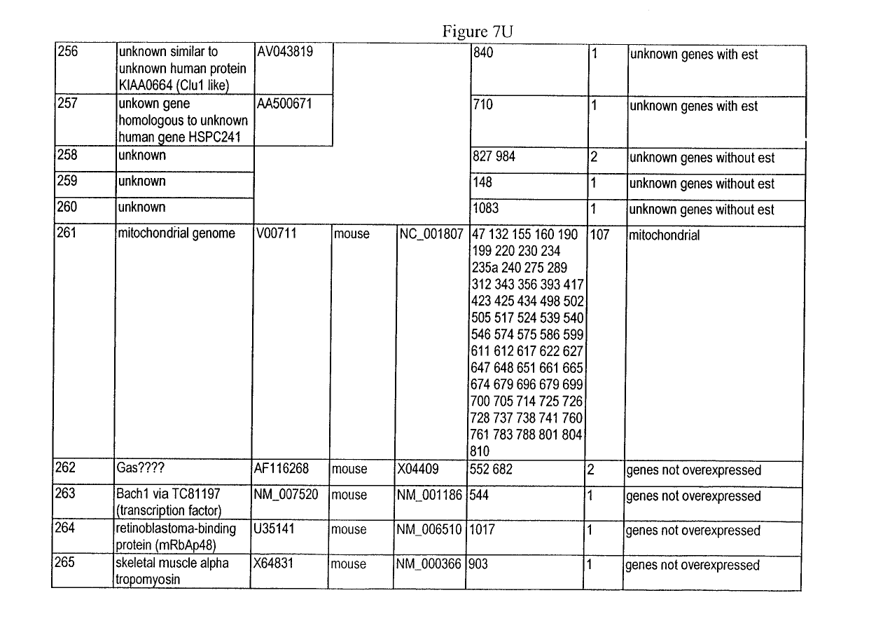

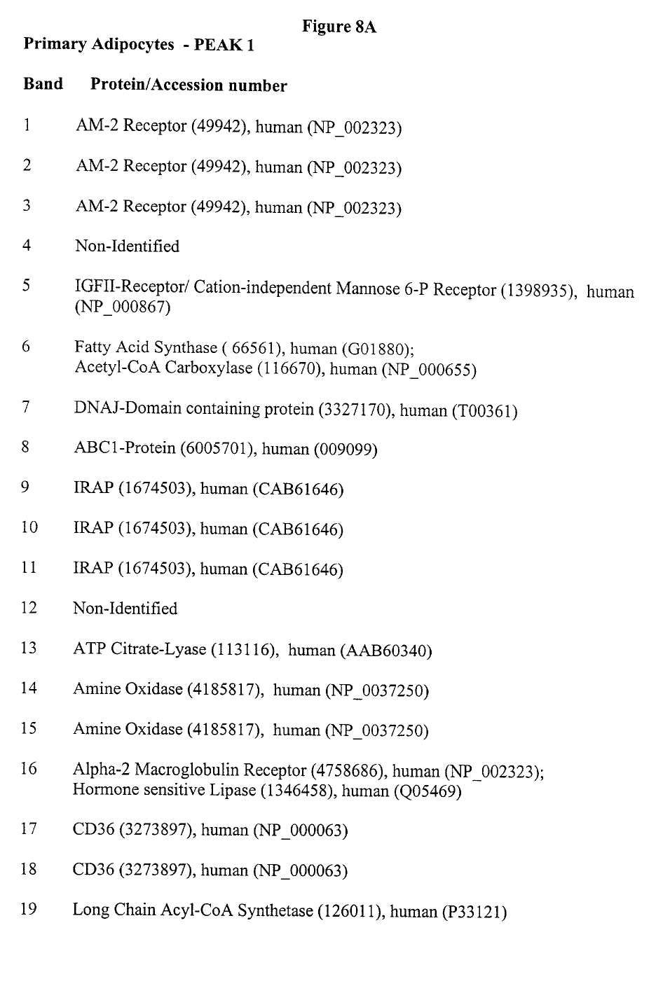

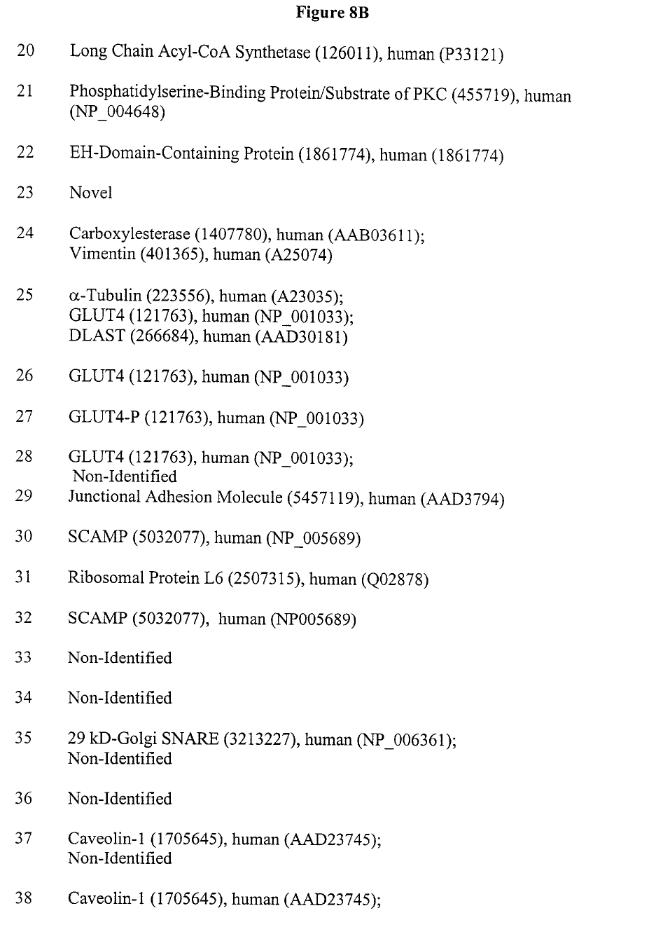

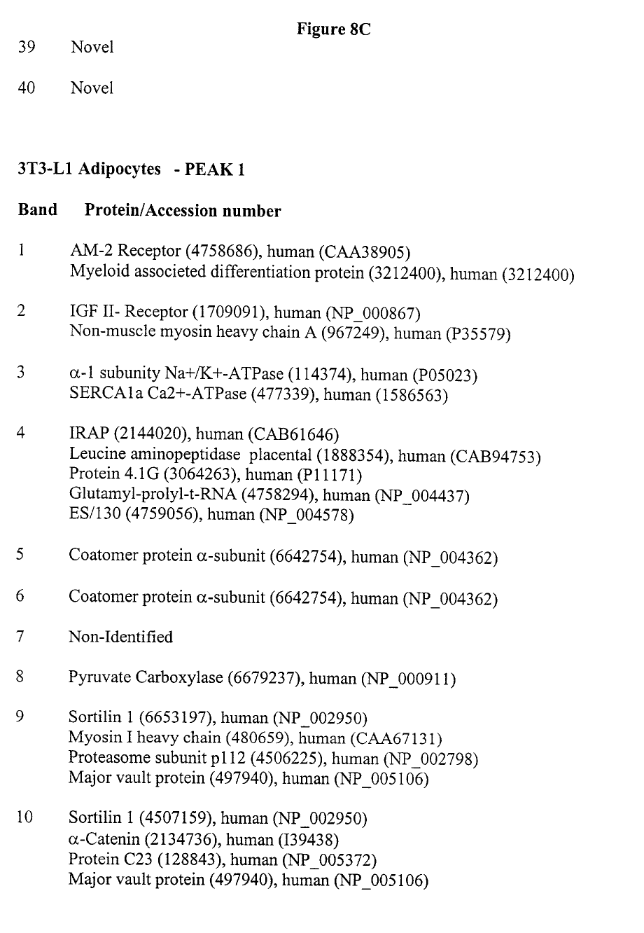

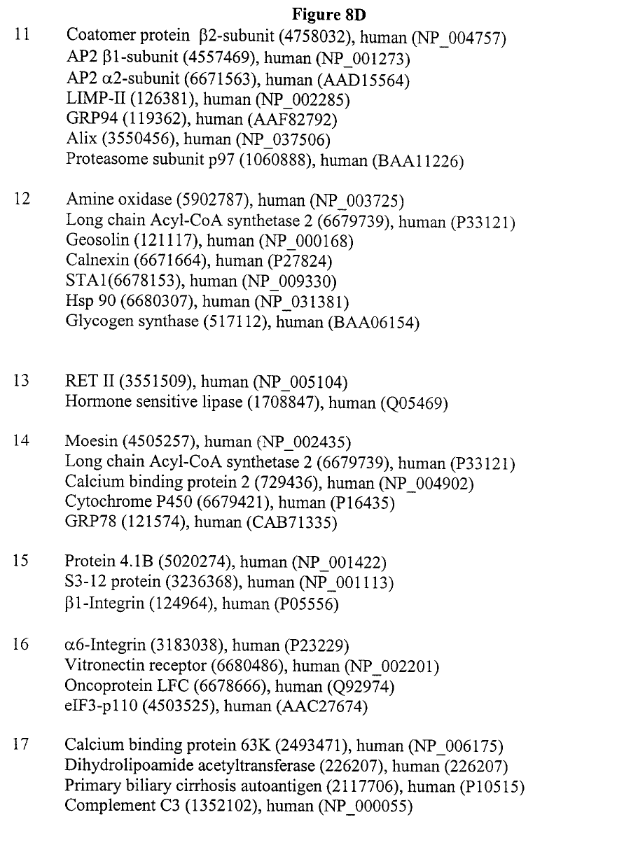

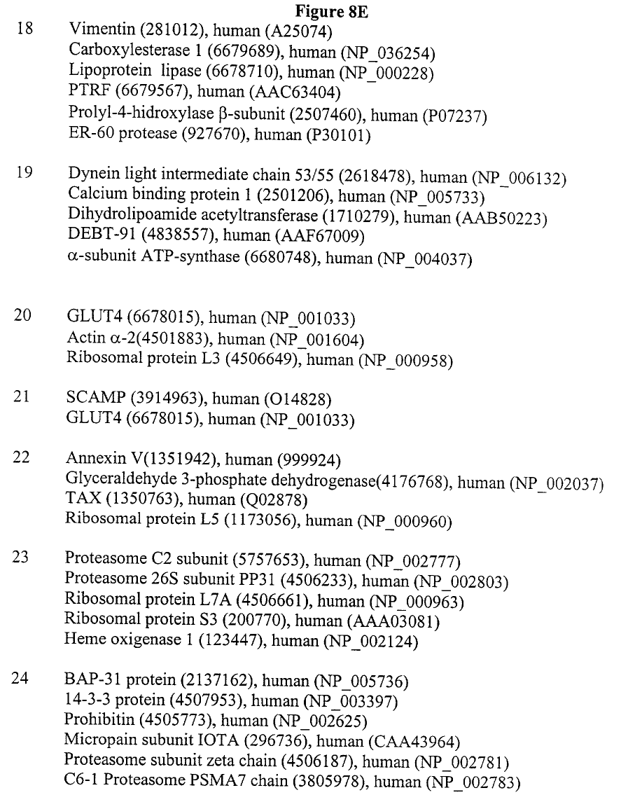

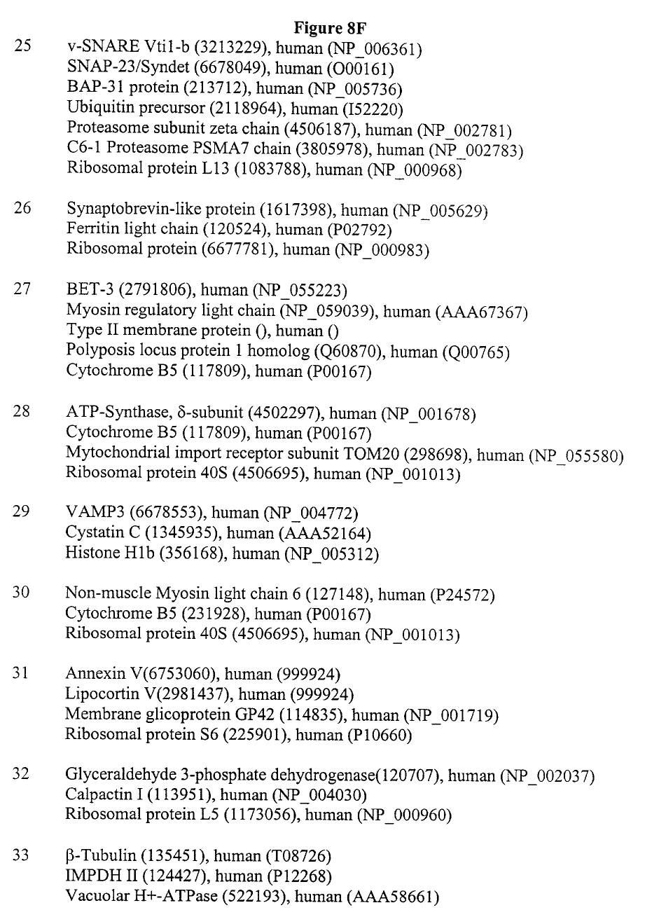

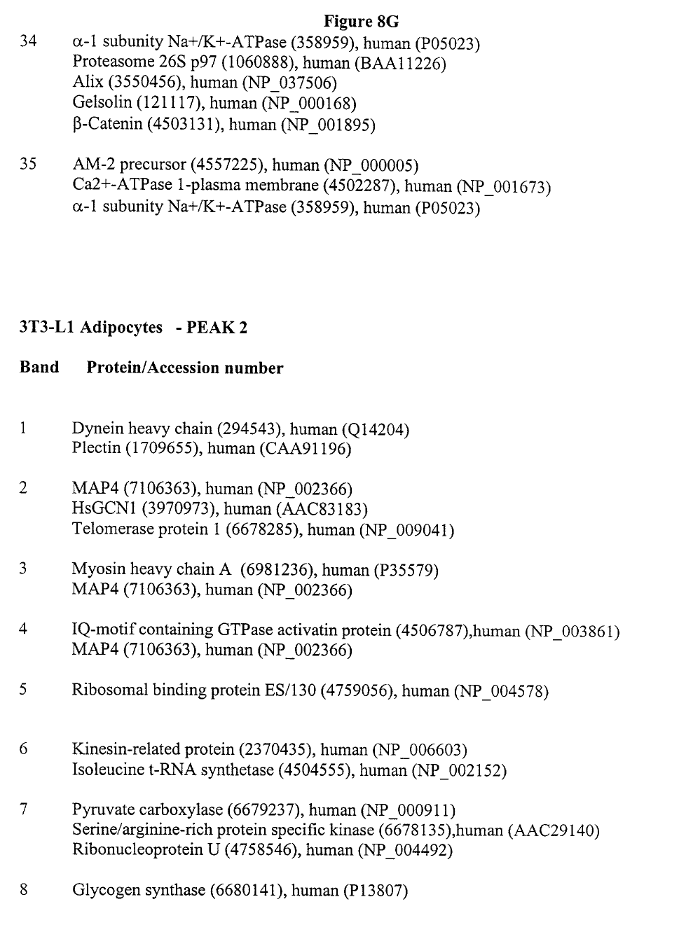

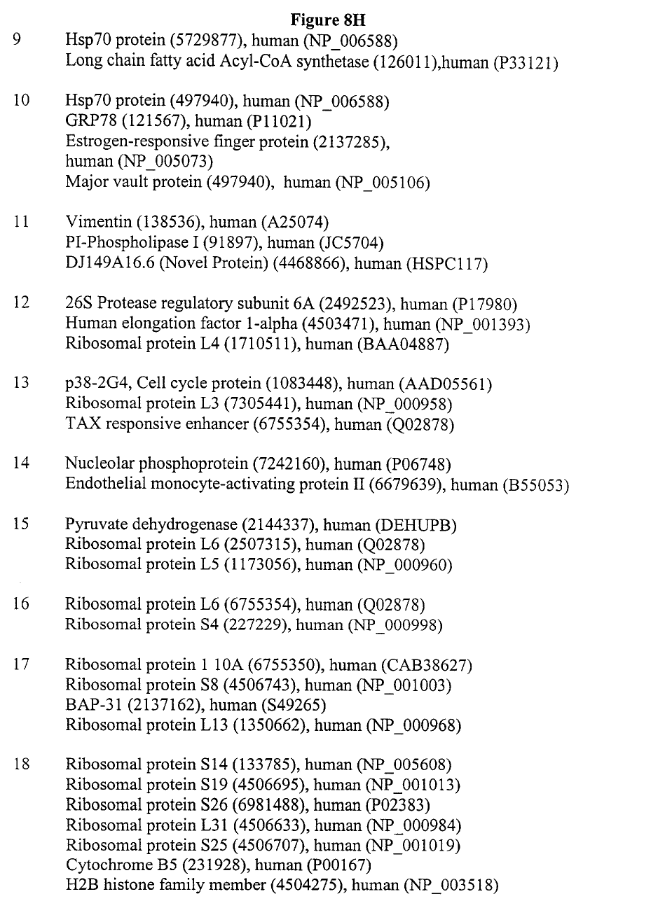

GLUT4-containing membranes were isolated by velocity sedimentation, then further fractionated using sucrose density equilibrium gradients, and, as described above, GLUT4-containing fractions that exhibited the most insulin sensitivity (peak 1; fraction 7-8 and the fractions containing GLUT4 that were less insulin sensitive (when compared to the peak fractions) were identified. The biogenesis of the peak 1 vesicle fraction was also observed to increase during 3T3-L1 adipocyte differentiation. To identify proteins present in GLUT4-containing vesicles, fractions corresponding to peak 1 from primary adipocytes, peak 1 from 3T3-L1 adipocytes, and peak 2 from 3T3-L1 adipocytes were pooled, subjected to SDS-PAGE and silver stained. The protein bands were subjected to tryptic hydrolysis and the peptides analyzed by mass spectrometry using standard techniques. FIGS. 8A-8I are a list of the peptides identified in peaks 1 and 2, as well as their GenBank Accession numbers and the Genbank Accession numbers of a human homolog if one is available.

These proteins are useful as targets for compounds that modulate glucose transport as well as for diagnosis of individuals having or at risk for disorders related to glucose transport.

### Example 8

Comparison of Muscle-adipocyte Union Library Sequences and GLUT4-enriched Vesicle Sequences

A comparison was made between the glucose transport-related proteins identified in the subtractive and the Adipocyte Union libraries and glucose transport-related proteins identified in glucose transport vesicles. FIG. 9 lists those proteins that were in common between at least one of the libraries and were also identified in peak 1 or 2 of the vesicle preparation. Acetyl-CoA carboxylase, carboxylesterase, caveolin-1, CDC36, are listed in this figure although their presence in peak 1 or peak 2 is not confirmed.

### Example 9

Analysis of Gene Expression Using DNA Arrays

DNA arrays can be used to assay the levels of gene expression of selected gene sequences. These were measured by assaying the amount of mRNA for the gene sequences selected for analysis in undifferentiated 3T3 L1 fibroblasts and differentiated 3T3 L1 adipocytes. The sequences selected for analysis are selected from the MAU library. Clones from the library that show significantly different levels of expression in differentiated adipocytes are selected for further analysis of their role in glucose transport.

A Protocol for Analyzing an Array Follows

1. Clones that are previously sequenced are selected from the MAU library. These clones consist of known and unknown genes with various levels of expression in fibroblasts and adipocytes.

2. Each of the clones is diluted 1:50 and then amplified by PCR.

3. PCR fragments are gel purified and re-suspended in 20-30 μl of ddH₂O.

4. Nucleic acid concentration of the PCT products is measured by spectrophotometer (OD₂₆₀) and further dilutions are made bringing all samples to a concentration of 100 ng/μl.

5. The PCR samples are then dot blotted (i.e., each to a separate address) onto a charged nylon membrane at 50 ng per dot as described in steps a-c.

a. The PCR samples are diluted to the desired concentration in 0.2 M NaOH/10 mM EDTA (denaturation solution) and then incubated at 37° C. for fifteen minutes.

b. The nylon membranes are pre-wetted and placed into a dot blot apparatus. Suction is applied to the apparatus and buffer is washed through the openings.

c. After denaturation the DNA solution is place in the apparatus (each sample in a separate well) and suction is applied. Once the solution has gone through the filter, the wells are washed with additional denaturation solution. The membrane is then removed from the apparatus and cross-linked with UV-radiation. Membranes are then baked to dryness and stored in sealed bags until ready for use. The membrane with the PCR sample is referred to as an array.

6. To analyze expression, the arrays are pre-hybridized for at least five minutes in modified Church's buffer (7% SDS, ImMEDTA, 0.5 M NaHP04 pH 7.2).

7. Probes for the arrays are labeled in a modified first strand cDNA synthesis reaction as follows:

a. Two labeling reactions are carried out side by side. One using adipocyte mRNA as the substrate and using fibroblast mRNA as the substrate.

b. For each labeling reaction, 2 μg of mRNA is combined with 2 μl of oligo d(T) and 2 μl of random hexamer and incubated at 70° C. for 10 minutes and then chilled on ice.

c. After the incubation, add 4 μl of 5×first strand buffer, 2 μl of 0.1 M dithiothreitol (DTT), and 1 μl of a modified cNTP solution (A, T, and G at 500 μM final; C at 5 μM final), and 5 μl of labeled dCTP. Mix, microfuge, and place at 37° C. for 2 minutes.

d. Add reverse transcriptase (2 μl Superscript II; Life Technologies Inc.; Rockville, Md.), mix and place at 32° C. for one hour.

e. Place on ice to stop reaction.

8. Unincorporated dNTPs are removed from the probe mixture by passing the mixture through a G50-150 Sephadex column (Sigma) and centrifuging for 1 minute at 1000×g.

a. To the labeling reaction add 1 μl 1% SDS, 1 μl 0.5M EDTA, and 3 μl 3M NaOH and incubate at 68° C. for three minutes and then at room temperature for fifteen minutes.

b. Add 10 μl of 1 M Tris-HCl pH 7.5 and 3 μl of 2N HCl.

c. Add an additional 50 μl of TEN (10 mM Tris-Cl, 1 mM EDTA, 100 mM NaCl, pH 8.0) buffer to the tube and filter the labeled mix through a G50-G150 Sephadex column to remove unincorporated nucleotides.

d. Add 50 μl of Cot1 DNA (Life Technologies Inc.; Rockville, Md.) to this mixture; boil for five minutes, and hold at 68° C. until ready to use.

9. The probe is added to a sufficient volume of modified Church's buffer and the mixture is added to the filters (add approximately the same number of counts to each array) and hybridized overnight at 65° C. with gentle rocking.

10 After hybridization the filters are washed as follows: twice at room temperature with 2×SSC/0.05% SDS for five minutes, once at room temperature with 0.1×SSC/0.1% SDS for ten minutes and finally once or twice at 65° C. with 0.1×SSC/0.1% SDS for 1 hour.

11 The damp arrays are wrapped in plastic wrap and put on a phosphor-imaging screen overnight (Filters may also be placed on auto-rad film).

12 Commercially available programs for phosphor-imagers quantify images. Alternatively the images can be quantified with commercially available graphics or image analysis programs. The quantified values represent the relative amount of expression of each sequence on the array.

13 The values are further analyzed by subtracting background from each measurement and the values are then graphically represented to facilitate comparisons between the values for fibroblast and for adipocytes.

This method allows for screening of multiple sequences in a single procedure. Such methods are useful for analyzing expression profiles in individuals having or at risk for a disorder related to glucose transport, for analyzing the ability of a test agent or a candidate agent to alter expression of a gene involved in glucose transport, and to analyze compounds that may be useful as drugs for other disorders for potential (deleterious) side effects resulting from unintended alterations in expression of genes involved in glucose transport.

Similar methods of analysis using arrays can be used for diagnostic purposes. For example, expression of sequences encoding proteins involved in glucose transport can be analyzed using a nucleic acid sample from the cells of an individual suspected of having a glucose transport-related disorder (e.g., type II diabetes). In general, the nucleic acid sample will represent sequences expressed in a cell type that conducts glucose transport. The sequences analyzed include sequences more highly expressed in adipocytes and/or muscle cells than in fibroblasts (including sequences expressed in adipocytes and/or muscle cells and having no detectable expression in fibroblasts). Such sequences are described herein. The level of expression of the sequences represented in the array is compared to a reference level of expression (representing the amount of expression present in an unaffected individual who is not at risk for the disorder). An alteration in the level of expression of one or more of the sequences indicates that the individual has or is at risk for the glucose transport-related disorder. The array may include one or more sequences that are used as standards (i.e., reference sequences) to normalize the data between reactions. In general, the sequences used as standards correspond to genes whose expression is not affected in glucose transport disorders. Sequences used as standards can also correspond to genes that are not differentially expressed between adipocytes, muscle cells, and fibroblasts. Examples of such sequences are described herein.

### Example 10

Genechip Identification of Genes Not Expressed in 3T3-L1 Fibroblast, but Present in 3T3-L1 Adipocytes and Muscle

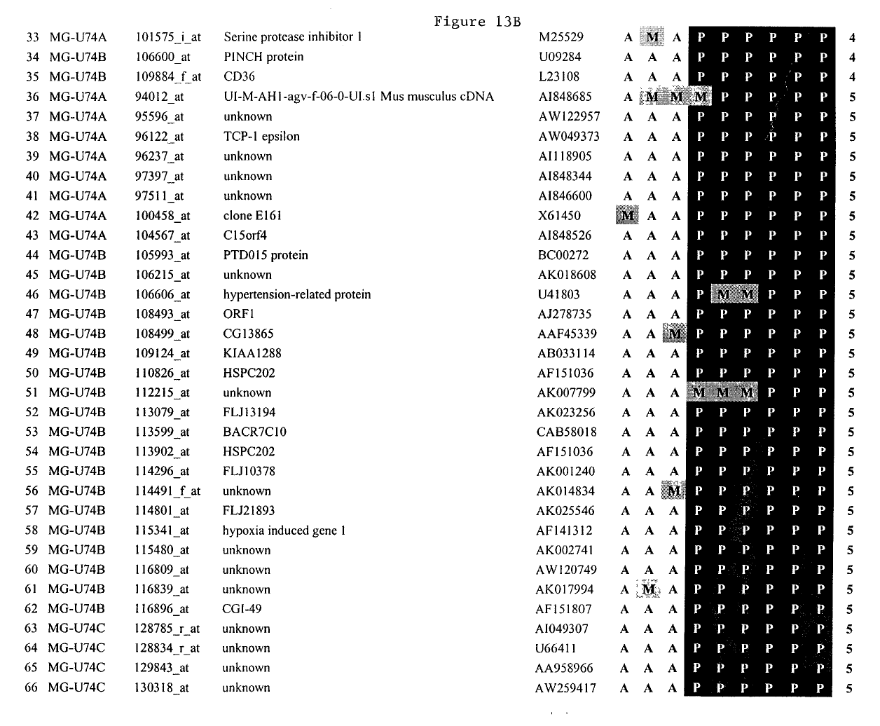

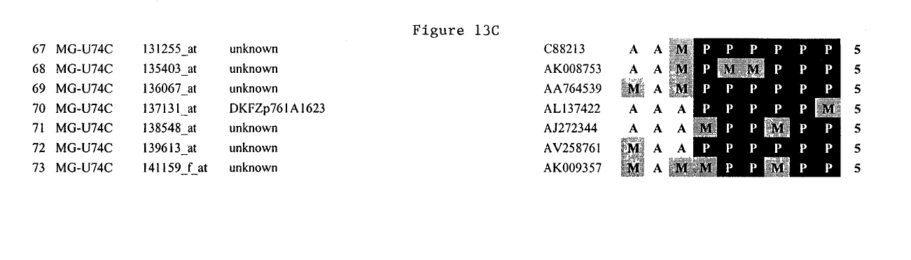

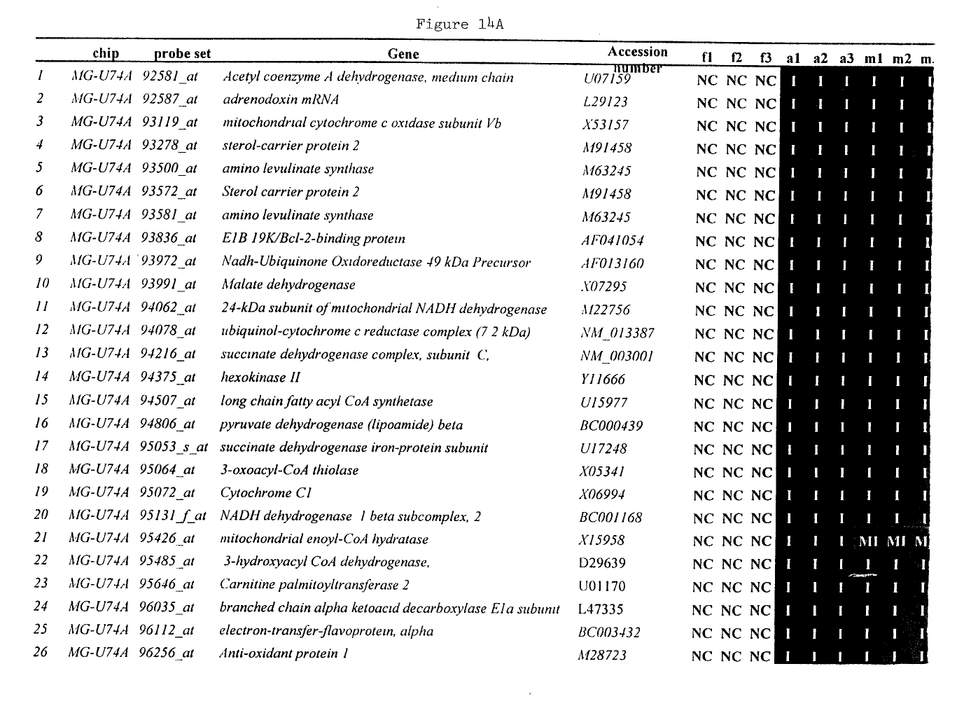

To further identify genes that are preferentially expressed in cells conducting glucose transport, the mouse U74A Genechip (Affymetrix) was probed with two independently produced sets of probes from 3T3-L1 fibroblast, 9 day old 3T3-L1 adipocytes, and mouse muscle. The experiments were carried out using standard methods, essentially as described above. The genes listed in FIGS. 13A-13C are those whose expression was not detected in fibroblasts, and was detected in adipocyte or muscle on one or both of the duplicate Genechips based on the Absolute call of gene expression made by the Affymetrix Microarray Suite Software. The columns in FIGS. 13A-13C marked f1 and f2 are data from the fibroblast replicate chips. The columns marked a1 and a2 are data from the adipocyte replicate chips, and the columns marked m1 and m2 are data from the muscle replicate chips. A indicates that the gene is absent in a tissue. P indicates that the gene is present in a tissue. An M indicates marginal signal and the software cannot determine if the gene is absent or present. The function classes of proteins listed in the last column are: Class 1 are genes encoding metabolic proteins; Class 2 are genes encoding signaling proteins; Class 3 are genes encoding cytoskeletal or trafficking proteins; and Class 4 are other proteins whose function is something other than those of Classes 1-3; and Class 5 are proteins of unknown function. Genes in italics encode mitochondrial proteins.

Genes that are expressed in adipocyte and/or muscle and are not expressed in fibroblasts are useful, e.g., for identifying genes whose expression is altered in disorders involving glucose transport, for detecting aberrations in glucose transport, and as targets for drugs designed to alter glucose transport. Genes that are expressed in both fibroblasts and adipocytes and/or muscle cells are also useful as reference sequences, e.g., to normalize data obtained when measuring expression patterns of genes expressed in glucose transport in a sample.

### Example 11

Probe Sets on Affymetrix GeneChip U74A whose Expression is Increased in both 3T3-L1 Adipocytes and Muscle Compared to Fibroblasts.

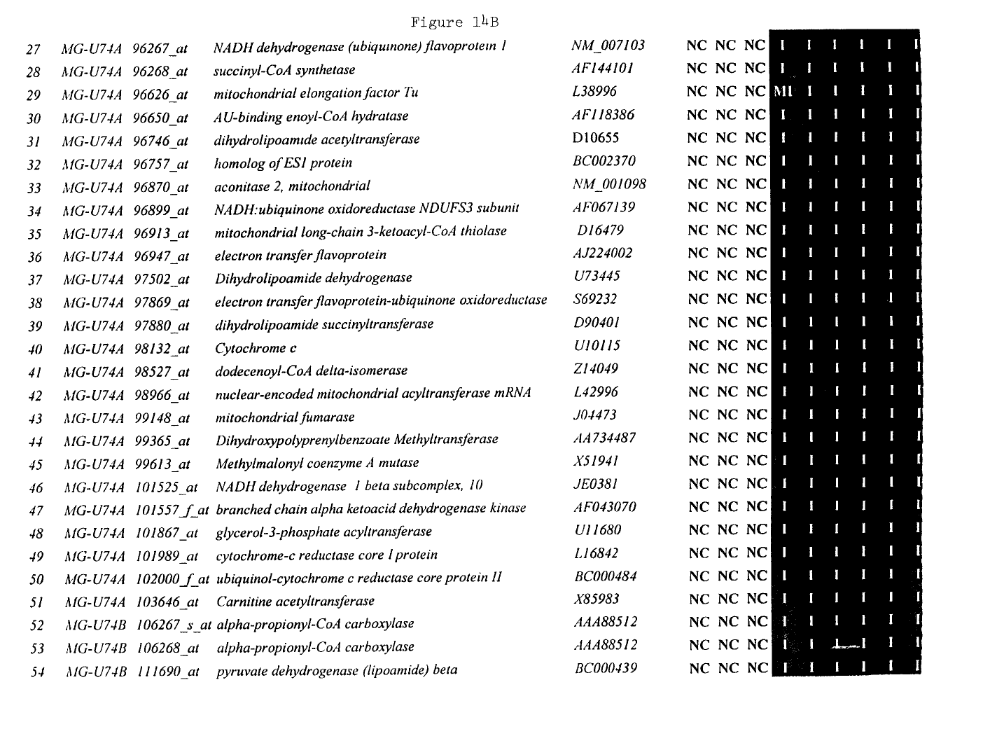

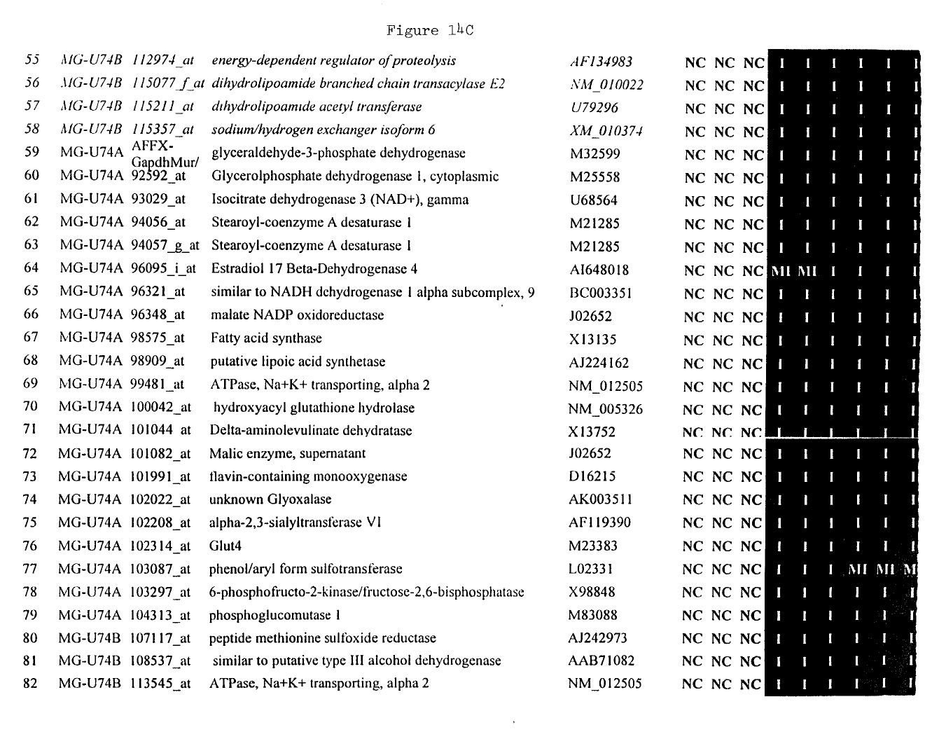

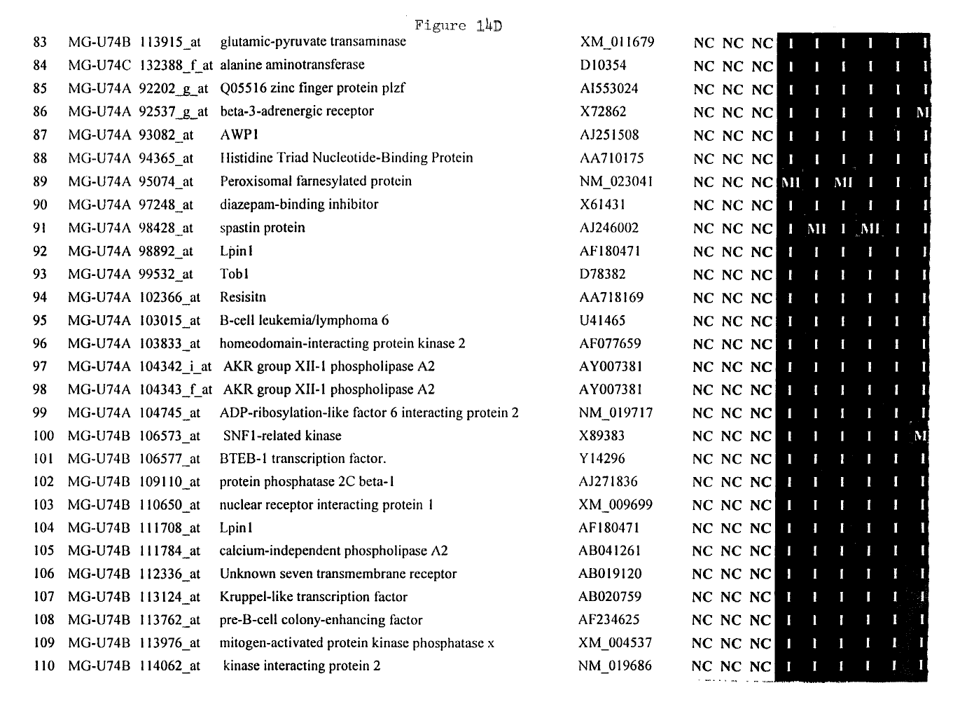

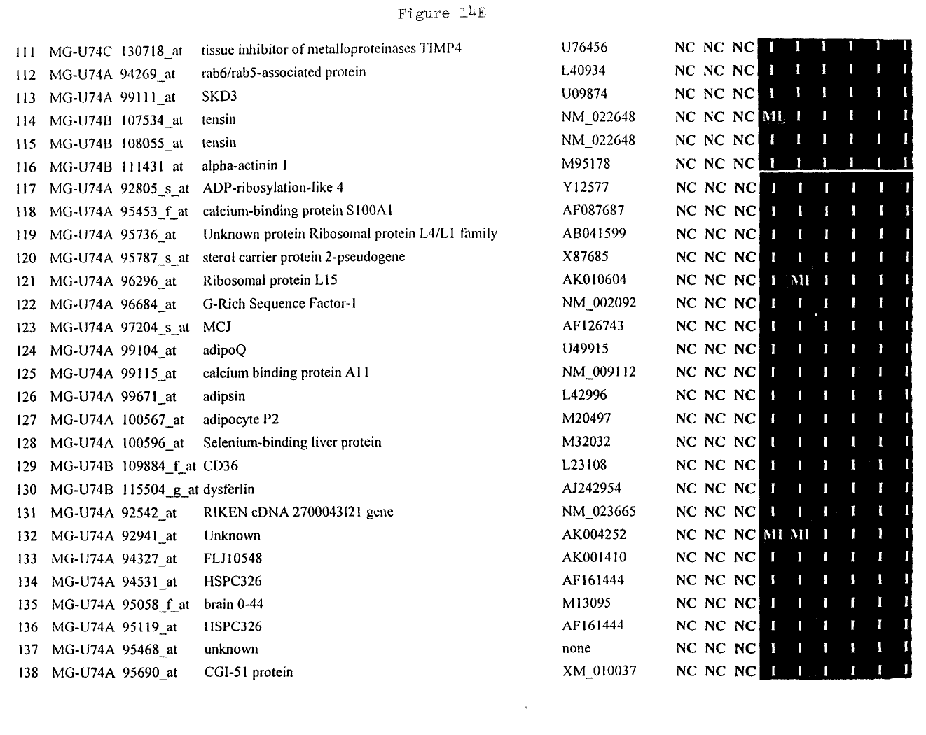

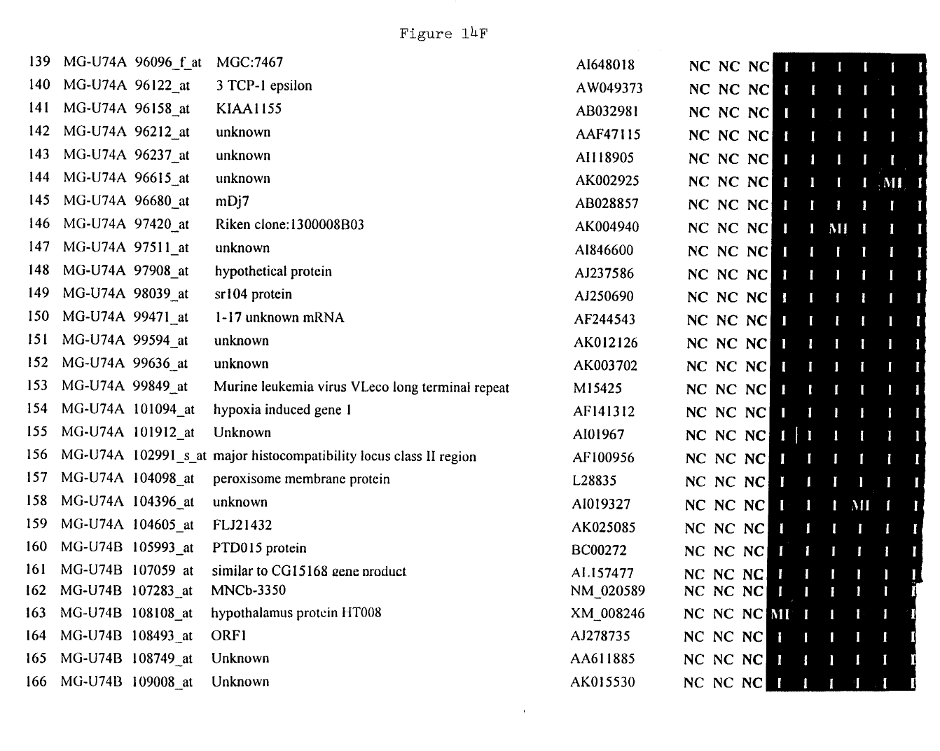

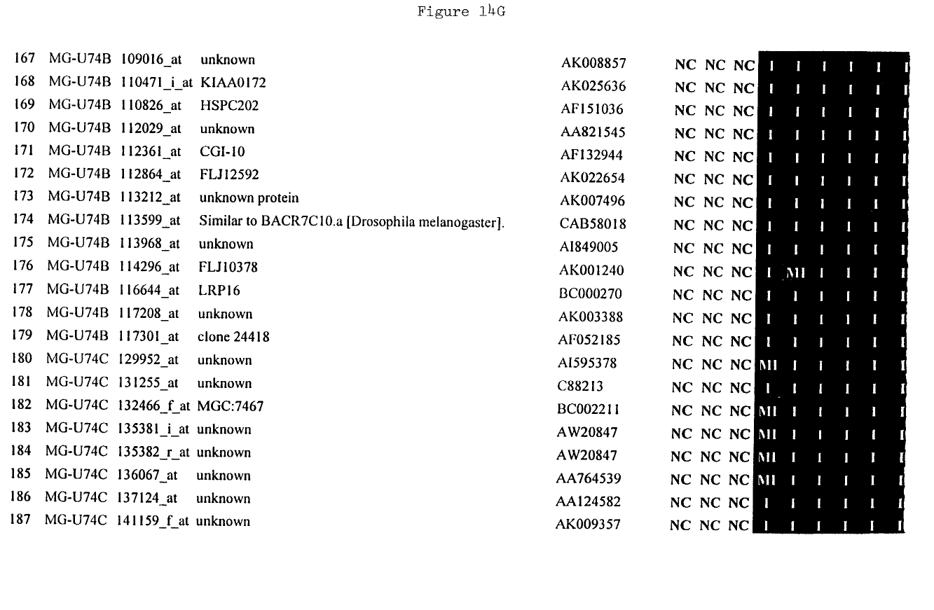

To determine the relative expression levels of genes in cells that conduct glucose transport compared to cells that do not conduct glucose transport, the mouse U74A GeneChip was probed with three independently produced cDNA probes from 3T3-L1 fibroblasts, 9 day old 3T3-L1 adipocytes, and mouse muscle. The experiments were conducted using standard methods, essentially as described above. The genes listed in FIGS. 14A-14G are those whose expression was determined to be the same on all fibroblast chips, and increased on both adipocyte or muscle GeneChips based on the difference change of gene expression made by the Affymetrix Microarray Suite Software when compared to the first fibroblast chip. The columns marked f1, f2, and f3 are fibroblast replicate chips. The columns marked a1, a2, and a3 are adipocyte replicate chips, and the columns marked m1, m2, and m3 are the muscle replicate chips. NC indicates no change of expression. MI indicates that there was a moderate increase in expression. An I indicates an increase in expression. The function classes of the genes listed in the last column are as follows: Class 1 genes encode metabolic proteins; Class 2 genes encode signaling proteins; Class 3 genes encode cytoskeletal or trafficking proteins; Class 4 genes encode proteins with functions other than those of Classes 1-3; and Class 5 are proteins of unknown function. Genes listed in italics encode mitochondrial proteins.

Genes with increased expression in adipocyte and/or muscle compared to fibroblasts are candidate genes for a glucose transport pathway. Such genes are useful, e.g., for identifying genes whose expression is altered in disorders involving glucose transport, detecting aberrations in glucose transport (e.g., for diagnostic purposes), and as targets for drugs designed to alter glucose transport. Genes whose expression is the same in fibroblasts and adipocytes and/or muscle cells are also useful as reference sequences, e.g., to normalize data obtained when measuring expression patterns of genes expressed in glucose transport in a sample.

In selecting nucleic acid sequences for the uses described herein, any of the genes or sequences identified using any of the above methods (i.e., subtraction libraries, vesicle proteins, or microarrays) can be combined. Particularly useful are those sequences corresponding to genes found to be preferentially expressed in adipocytes or muscle cells compared to fibroblasts in at least two of the methods. In some embodiments, the sequences are selected from those that are preferentially expressed in both adipocytes and muscle cells compared to their expression in fibroblasts in at least two of the methods.

### Example 12

Assay for GLUT4 Transport/insulin Mediated Transport

Methods are available for the rapid testing of the functions of proteins identified as glucose transport-related proteins, e.g., by assaying their role in GLUT4 regulation. For example, a reporter molecule that is a chimera of the transferrin receptor (exofacial domain) and the IRAP (insulin-regulated aminopeptidase) protein that traffics in cells like GLUT4 has been described as a surrogate for GLUT4 (Johnson et al., 2001, Mol. Biol. Cell 12:367-381; Lampson et al., 2000, J. Cell Sci. 113:4065-4076; Subtil et al., 2000, J. Biol. Chem. 275:4787-95; Johnson et al., 1998, J. Biol. Chem. 273:17968-17977). This chimera is expressed in cells and is sequestered in the perinuclear region under basal conditions. Insulin then stimulates the chimera's translocation to the cell surface. The translocation can be readily measured using an antibody raised against the exofacial domain of the transferrin receptor or by labeled transferrin itself. This assay is then applied to cells in which the protein of interest (e.g., a glucose transport-related protein) has altered expression. For example, the protein of interest can be overexpressed in a cell that also expresses the transferring/IRAP chimera, and the effect of overexpression on insulin regulation of translocation assayed. This assay can also be used to determine if a test agent or candate agent targeted to a glucose transport-related protein is an effective modulator of insulin regulation of translocation. For example, the candidate agent can be a ribozyme or antisense sequence that is targeted to a nucleic acid sequence encoding a glucose transport-related protein, e.g., RabGAP or endophilin 1b. Similarly, the assay can be performed in the presence and absence of a candidate agent targeted to a glucose transport-related protein or nucleic acid sequence. An alteration in transport of the chimera in the presence of the candidate agent indicates that it is a candidate agent, useful for treating a disorder associated with aberrant glucose transport (e.g., type II diabetes).

Two examples of genes identified using the methods described herein that can be used in the assay methods described above are those encoding an apparent RabGAP and endophilin 1b. The RabGAP protein is predicted to be a negative regulator of Rab GTPases, which are known to promote membrane recycling of GLUT4 as it transits from intracellular storage sites to the plasma membrane and back into the cell. One such protein, Rab 4, is implicated in directing GLUT4 to its perinuclear recycling compartment, a necessary step for GLUT4 to respond to insulin. The RabGAP that was identified is predicted to inhibit Rab 4 by increasing the GTPase activity of Rab 4 leading to its binding GDP and deactivation. Thus, RabGAP is an excellent drug target in that its inhibition might lead to promoting Rab4, a required element in the regulation of GLUT4 by insulin. Endophilin 1b is related to a class of brain endophilin proteins that are involved in promoting endocytosis of plasma membrane proteins. The high expression of endophilin 1b in adipocytes indicates that it is likely to be involved in endocytosis of GLUT4 in these cells. Endophilin 1b is therefore another potential drug target in that its inhibition by a drug is predicted to retain GLUT4 at the cell surface membrane where it can promote glucose transport, thereby lowering blood glucose.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of identifying a gene whose expression is altered in a glucose transport-related disease or disorder, the method comprising:
providing a nucleic acid array comprising 4 or more nucleic acids immobilized on a solid support, each nucleic acid comprising a sequence of 10 or more consecutive nucleotides within any one of the sequences listed in FIGS. 1, 2A-2R, 3A-3E, 6A-6E, 7A-7U, 8A-8I, 13A-13C, and 14A-14G or a complement thereof;
providing a reference nucleic acid sample prepared from a tissue of a normal, control mammal;
contacting the array with the reference sample;
detecting hybridization of the reference sample with nucleic acids in the array, to obtain a reference pattern of glucose transport-related gene expression;
providing a test nucleic acid prepared from a tissue of a mammal having a glucose transport-related disease or disorder;
contacting the array with the test sample;
detecting hybridization of the test nucleic acid with nucleic acids in the array, to obtain a test pattern of glucose transport-related gene expression; and
comparing the reference pattern with the test pattern to detect a gene whose expression is altered in the test pattern relative to its expression in the reference pattern.

2. The method of claim 1, wherein the array comprises 10 or more nucleic acids.

3. The method of claim 1, wherein the array comprises 100 or more nucleic acids.

4. The method of claim 1, wherein the array comprises not more than 100 nucleic acids.

5. The method of claim 1, wherein the array comprises not more than 200 nucleic acids.

6. The method of claim 1, wherein the array comprises not more than 300 nucleic acids.

7. The method of claim 1, wherein the sequence comprises 30 or more nucleotides.

8. The method of claim 1, wherein the reference nucleic acid and the test nucleic acid are cDNAs.

9. The method of claim 8, wherein the cDNAs comprise a fluorescent label.

10. A nucleic acid array comprising 4 or more nucleic acids immobilized on a solid support, each nucleic acid comprising a sequence of 10 or more consecutive nucleotides within any one of sequences listed in FIGS. 1, 2A-2R, 3A-3E, 6A-6E, 7A-7U, 8A-8I, 9, 13A-13C, and 14A-14G.

11. The array of claim 10, wherein the array comprises 100 or more nucleic acids.

12. The array of claim 10, wherein the array comprises not more than 100 nucleic acids.

13. The array of claim 10, wherein the array comprises not more than 200 nucleic acids.

14. The array of claim 10, wherein the array comprises not more than 300 nucleic acids.

15. An isolated nucleic acid molecule comprising a nucleotide sequence selected from the group consisting of SEQ ID NOS: 1-3, or a complement thereof.

16. A nucleic acid molecule of claim 15, consisting of a nucleotide sequence selected from the group consisting of SEQ ID NOS: 1-3, or a complement thereof and a non-nucleic acid modifying group bound to either a 3′ or 5′ end of the nucleotide sequence or both.

17. A nucleic acid molecule of claim 15, consisting of a nucleotide sequence selected from the group consisting of SEQ ID NOS: 1-3, or a complement thereof, and a synthetic nucleic acid sequence bound to a 3′ or 5′ end of the nucleic acid sequence or both.

18. An isolated polypeptide comprising an amino acid sequence encoded by a nucleic acid sequence selected from the group consisting of SEQ ID NOS: 1-3.

19. An isolated nucleic acid molecule comprising a nucleic acid sequence selected from the group consisting of SEQ ID NOS: 4-93, or a complement thereof.

20. A nucleic acid molecule of claim 19, consisting of a nucleotide sequence selected from the group consisting of SEQ ID NOS: 4-93, or a complement thereof and a non-nucleic acid modifying group bound to either a 3′ or 5′ end of the nucleotide sequence or both.

21. A nucleic acid molecule of claim 19, consisting of a nucleotide sequence selected from the group consisting of SEQ ID NOS: 4-93, or a complement thereof, and a synthetic nucleic acid sequence bound to a 3′ or 5′ end of the nucleic acid sequence or both.

22. An isolated nucleic acid molecule of claim 19, consisting of a nucleic acid sequence selected from the group consisting of SEQ ID NOS: 4-93, or a complement thereof.

23. An isolated polypeptide comprising an amino acid sequence encoded by a nucleic acid sequence selected from the group consisting of SEQ ID NOS: 4-93.

24. A method for identifying a candidate agent, that modulates the expression or activity of a glucose transport-related polypeptide, the method comprising:
a) providing a sample containing a glucose transport-related polypeptide;
b) adding a test agent to the sample;
c) assaying the sample for expression or activity of the glucose transport-related polypeptide; and
f) comparing the effect of the test agent on expression or activity of the glucose transport-related polypeptide relative to a control, wherein a change in glucose transport-related polypeptide expression or activity indicates that the test agent is a candidate agent that can modulate expression or activity of the glucose transport-related polypeptide.

25. The method of claim 24, wherein the test agent is selected from the group consisting of a polynucleotide, a polypeptide, a small non-nucleic acid organic molecule, a small inorganic molecule, and an antibody.

26. The method of claim 24, wherein the test agent is selected from the group consisting of an antisense oligonucleotide and a ribozyme.

27. The method of claim 24, wherein the glucose transport-related polypeptide is assayed using an antibody.

28. The method of claim 24, wherein the glucose transport-related polypeptide is a human glucose transport-related polypeptide.

29. The method of claim 24, wherein the method comprises the step of determining whether glucose transport is modulated in the presence of the test agent.

30. The method of claim 29, wherein glucose transport is decreased in the presence of the test agent.

31. The method of claim 29, wherein glucose transport is increased in the presence of the test agent.

32. The method of claim 24, wherein the assay is a cell based assay.

33. The method of claim 24, wherein the assay is a cell-free assay.

34. The method of claim 24, wherein the glucose transport-related polypeptide is selected from the group of polypeptides encoded by sequences comprising the nucleic acid sequences listed in FIGS. 1, 2A-2R, and 3A-3E, and the polypeptides listed in FIGS. 6A-6E, 7A-7U, 8A-8I, 9, 13A-13C, and 14A-14G 6-9.

35. A method for identifying a candidate agent that modulates expression of a glucose transport-related polynucleotide, the method comprising:
a) providing a sample in which a glucose transport-related polynucleotide is expressed;
b) adding a test agent to the sample;
c) detecting expression of the glucose transport-related polynucleotide;
d) determining the amount of expression of the glucose transport-related polynucleotide; and
e) comparing the effect of the test agent on the amount of expression of the glucose transport-related polynucleotide in the sample relative to a control, wherein a change in the amount of expression from the glucose transport-related polynucleotide indicates the test agent is a candidate agent that can modulate expression of the glucose transport-related polynucleotide.

36. The method of claim 35, wherein the test agent is selected from the group consisting of a polynucleotide, a polypeptide, a small non-nucleic acid organic molecule, a small inorganic molecule, and an antibody.

37. The method of claim 35, wherein the test agent is selected from the group consisting of an antisense oligonucleotide and a ribozyme.

38. The method of claim 35, wherein the glucose transport-related polynucleotide is a human glucose transport-related polynucleotide.

39. The method of claim 35, wherein the method comprises the step of determining whether glucose transport is modulated in the presence of the test agent.

40. The method of claim 39, wherein glucose transport is decreased in the presence of the test agent.

41. The method of claim 39, wherein glucose transport is increased in the presence of the test agent.

42. The method of claim 35, wherein the glucose transport-related polynucleotide is selected from the group of sequences listed in FIGS. 1, 2A-2R, and 3A-3E-3 or a complement thereof, and listed in FIGS. 6A-6E, 7A-7U, 8A-8I, 9, 13A-13C, and 14A-14G, or a complement thereof.

43. The method of claim 35, wherein the assay is a cell-based assay.

44. The method of claim 35, wherein the assay is a cell-free assay.

45. A method of diagnosing an individual having or at risk for a glucose transport-related disorder, the method comprising:
(a) providing a nucleic acid array comprising 4 or more nucleic acids immobilized on a solid support, each nucleic acid comprising a sequence of 10 or more nucleotides, the sequence comprising or containing a sequence selected from the group of the sequences listed in FIGS. 1, 2A-2R, and 3A-3E, or a complement thereof, and the sequences of the genes listed in FIGS. FIGS. 6A-6E, 7A-7U, 8A-8I, 9, 13A-13C, and 14A-14G, or a complement thereof;
(b) providing a nucleic acid sample from the individual;
(c) contacting the array with the sample from the individual
(d) detecting hybridization of nucleic acid in the sample from the individual with each nucleic acid in the array, to obtain a pattern of glucose transport-related gene expression;
(e) comparing the pattern of glucose transport-related gene expression in sample from the individual with a reference pattern, wherein a comparison of the pattern of expression in the individual compared to the reference pattern indicates whether the individual has or is at risk for a glucose transport-related disorder.

46. The method of claim 41, wherein the array comprises 10 or more nucleic acids.

47. The method of claim 41, wherein the array comprises 100 or more nucleic acids.

48. The method of claim 41, wherein the array comprises not more than 100 nucleic acids.

49. The method of claim 41, wherein the array comprises not more than 200 nucleic acids.

50. The method of claim 41, wherein the array comprises not more than 300 nucleic acids.

51. The method of claim 41, wherein the sequence comprises 30 or more nucleotides.

52. The method of claim 41, wherein the sample from the individual is a cDNA sample.

53. The method of claim 48, wherein the cDNA sample comprises a fluorescent label.

54. The method of claim 48, wherein the disorder is type II diabetes.

55. A nucleic acid array comprising 4 or more nucleic acids immobilized on a solid support, each nucleic acid comprising a sequence of 10 or more nucleotides, the sequence consisting of at least a portion of a sequence selected from the group consisting of the sequences listed in FIGS. 1, 2A-2R, and 3A-3E, or a complement thereof, FIGS. 6A-6E, 7A-7U, 8A-8I, 9, 13A-13C, and 14A-14G, or a complement thereof.
